SEQUENCE LISTING (5 sequences)

SEQ ID NO: 1 - DNA, 19 bp, synthetic construct
  misc_feature 1..19: note = Synthetic Sequence
  source 1..19: mol_type = other DNA
ttactaagcacaacactct

SEQ ID NO: 2 - DNA, 19 bp, synthetic construct
  source 1..19: mol_type = other DNA
tacacattactttgaatcc

SEQ ID NO: 3 - DNA, 19 bp, synthetic construct
  misc_feature 1..19: note = Synthetic Sequence
  source 1..19: mol_type = other DNA
aactgagaaactaatgcct

SEQ ID NO: 4 - DNA, 19 bp, synthetic construct
  source 1..19: mol_type = other DNA
ttcacagctacatcaacct

SEQ ID NO: 5 - DNA, 19 bp, synthetic construct
  misc_feature 1..19: note = Synthetic Sequence
  source 1..19: mol_type = other DNA
tttcttgcaaatgcattcg

(19) United States
(12) Patent Application Publication
(10) Pub. No.: US 2023/0212202 A1
(43) Pub. Date: Jul. 6, 2023

(54) TREATMENT OF MGMT DEFICIENT CANCER WITH 2-FLUOROETHYL-SUBSTITUTED NITROSOUREAS AND OTHER COMPOUNDS

(71) Applicant: YALE UNIVERSITY, New Haven, CT (US)

(72) Inventors: Seth Herzon, New Haven, CT (US); Ranjit Bindra, New Haven, CT (US); Kingson Lin, New Haven, CT (US)

(21) Appl. No.: 18/067,497

(22) Filed: Dec. 16, 2022

Related U.S. Application Data
(60) Provisional application No. 63/290,611, filed on Dec. 16, 2021.

(51) Int. Cl. C07F 9/48; C07D 207/46; C07D 239/42; C07C 275/68; A61P 35/00

(52) U.S. Cl. CPC C07F 9/4816; A61P 35/00; C07C 275/68; C07D 207/46; C07D 239/42

(57) ABSTRACT

Disclosed are nitrosourea and other compounds, pharmaceutical composition, and methods of treating cancers that are MGMT deficient regardless of their MMR status and particularly compounds, pharmaceutical compositions, and methods of treating cancers that are both MGMT and MMR deficient.

### CROSS-REFERENCE TO RELATED APPLICATIONS

This application claims priority to U.S. Provisional Pat. Application No. 63/290,611, filed Dec. 16, 2021, the disclosure of which is incorporated herein by reference in its entirety.

### STATEMENT REGARDING FEDERALLY SPONSORED RESEARCH

This invention was made with government support under GM131913, GM007205, and CA254158 awarded by National Institutes of Health and under CA016359 awarded by National Cancer Institute. The government has certain rights in the invention.

### INCORPORATION-BY-REFERENCE OF MATERIAL SUBMITTED VIA THE OFFICE ELECTRONIC FILING SYSTEM

This disclosure contains one or more sequences in a computer readable format in an accompanying .xml file titled “047162-7385US1 (01869) Sequence Listing ST_26,” which is 6.51 KB in size and was created Dec. 16, 2022, the contents of which are incorporated herein by reference in their entirety.

### BACKGROUND

The following discussion is provided merely to aid the reader in understanding the disclosure and is not admitted to describe or constitute prior art thereto.

Nitrosoureas are DNA alkylators that have been used for over 40 years to treat cancer, particularly brain cancers such as gliomas. DNA alkylators act by alkylating the O⁶ guanine in a cell’s DNA, thus preventing effective replication and killing the cell. Such alkylators are effective only in cells which have below normal expression of the DNA repair protein MGMT (O⁶-methylguanine-DNA-methyltransferase). These cells are termed “MGMT deficient”. In cells which express normal levels of MGMT, the enzyme can reverse the alkylation and restore the affected DNA to its pre-alkylation status. Since expression of MGMT is frequently lost in tumorigenesis, monofunctional alkylators can differentially kill cancer cells that lack this repair protein while MGMT proficient non-cancerous cells can survive. This principle is known clinically as therapeutic index.

Known nitrosourea anti-cancer agents include carmustine, lomustine, nimustine, fotemustine, semustine, streptozocine, methyl nitrosourea (MNU), and methyl nitronitrosoguanidine (MNNG). These nitrosoureas and their synthesis and pharmaceutical activity are described, for example, in “Cancer Therapeutic Agents”, G.L. Finch, et al. in “Encyclopedia of Toxicology (third edition) 2014; “DNA-Interactive Agents”, Richard B Solomon et al. in “The Organic Chemistry of Drug Design and Drug Action (third edition) 2014;” DNA Alkylating Agents” Carmen Avendano et al. in “Medicinal Chemistry of Anticancer Drugs (second edition) 2015; “Antineoplastics” R. S. Vardanyan et al. in “Synthesis of Essential Drugs (2006); and Edward J. Hessler et al. - “Improved Synthesis of Streptozocine”, J. Org. Chem 1070, 35, 1, 245-246.

In addition to mutations in MGMT, cancers also often develop mutations in mismatch repair (MMR) genes, either as a consequence of treatment or during normal tumorigenesis. Because cancers having MMR mutations may become resistant to alkylators such nitrosoureas, such alkylators do not provide an effective therapeutic regimen for treating such cancers.

Compounds having an improved efficacy in the treatment of cancer, particularly those cancers that are MMR deficient, would be highly desirable.

### SUMMARY

The disclosure provides a method of treating an MGMT deficient cancer in a patient in need of such treatment. In certain embodiments, the method comprises administering to said patient a therapeutically-effective amount of a compound of formula (I) or a pharmaceutically-acceptable salt thereof:

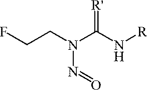

wherein R′ is selected from O and NH; and R is selected from H, NO₂, CH₂CH₂Cl, CH₂CH₂F, cyclohexyl, 4-methylcyclohexyl, —C(H)(CH₃)—P(═O)(OCH₂CH₃)₂,

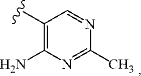

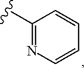

and

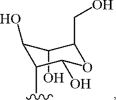

pharmaceutical compositions comprising a compound of formula (I) and a pharmaceutically-acceptable carrier, and compounds of formula (I).

The compounds of formula (I) and their pharmaceutically-acceptable salts are useful to treat, ameliorate, and/or prevent cancer that is MGMT deficient regardless of MMR status and are particularly useful to treat cancer that is both MGMT and MMR deficient.

### BRIEF DESCRIPTION OF THE FIGURES

The drawings illustrate generally, by way of example, but not by way of limitation, various embodiments of the present application.

FIGS. 1A-1F show an overview of mechanistic strategy and structures of agents. (FIG. 1A) Depiction of, without being bound by theory, the underlying mechanistic hypothesis, according to various embodiments. Systemic administration of a bifunctional agent is envisioned to form a primary lesion that is rapidly resolved by healthy (DDR+) but not DDR-deficient (DDR-) cells. The persistence of the primary lesion allows it to evolve slowly to a more toxic secondary lesion. (FIG. 1B) Shows that TMZ (1a) is the front-line therapy for the treatment of MGMT- GBM. Under physiological conditions, TMZ (1a) converts to MTIC (1b) which decomposes to methyl diazonium (1c). (FIG. 1C) Shows that O⁶-Guanine is the most clinically-significant site of methylation by methyl diazonium (1c). O⁶MeG (3) is rapidly reverted to dG (2) by MGMT (the second-order rate constant for demethylation of calf thymus DNA by MGMT is 1×10⁹ M⁻¹•min⁻¹), but persists in the genome of MGMT- cells, ultimately leading to MMR-dependent cytotoxicity. (FIG. 1D) Shows that the imidazotetrazine KL-50 (4a) can be a source of 2-fluoroethyl diazonium ion (4c). (FIG. 1E) Shows that fluoroethylation at O⁶-guanosine would form O⁶FEtG (5), which is known to slowly rearrange (t_{½} ~ 18.5 h at 37° C.) via intermediate 6 to form the dG-dC ICL. Based on the broad substrate scope of MGMT, it was anticipated that O⁶FEtG (5) would be readily reversed in MGMT+ cells, thereby preventing ICL formation in healthy tissue. Realization of this goal would provide the first MMR-independent agent active specifically in MGMT- glioma. (FIG. 1F) Shows structures of the triazenes 9-13, mitozolomide 12a, and lomustine (CCNU, 14).

FIGS. 2A-2H show that KL-50 (4a) displays MGMT-dependent, MMR-independent cytotoxicity in multiple isogenic cell models. (FIG. 2A) Summary of IC₅₀ values derived from short-term viability assays in LN229 MGMT+/-, MMR+/- cells treated with TMZ (1a) derivatives. ^{a}MGMT TI (therapeutic index) = IC₅₀ (MGMT+/MMR+) divided by IC₅₀ (MGMT-/MMR+). ^{b}MMR RI (resistance index) = IC₅₀ (MGMT-/MMR-) divided by IC₅₀ (MGMT-/MMR+). (FIG. 2B) Short-term viability assay curves for TMZ (1a), CCNU (14), KL-85 (4b), and KL-50 (4a) in LN229 MGMT+/-, MMR+/- cells. (FIG. 2C) Clonogenic survival curves for TMZ (1a) in LN229 MGMT+/-, MMR+/- cells, with representative images of wells containing 1000 plated cells treated with 30 µM TMZ (1a). (FIG. 2D) Clonogenic survival curves for KL-50 (4a) in LN229 MGMT+/-, MMR+/- cells, with representative images of wells containing 1000 plated cells treated with 30 µM KL-50 (4a). (FIG. 2E) Short-term viability assay curves for TMZ (1a) in DLD1 MSH6-deficient cells pre-treated with 0.01% DMSO control (CTR) or 10 µM O⁶BG (+O⁶BG) for 1 h prior to TMZ (1a) addition to deplete MGMT. (FIG. 2F) Short-term viability assay curves for KL-50 (4a) in DLD1 MSH6-deficient cells pre-treated with 0.01% DMSO control (CTR) or 10 µM O⁶BG (+O⁶BG) for 1 h prior to KL-50 (4a) addition. (FIG. 2G) Short-term viability assay curves for TMZ (1a) in HCT116 MLH1-/- cells or HCT116 cells complemented with chromosome 3 carrying wildtype MLH1 (+Chr3) pre-treated with 0.01% DMSO control or 10 µM O⁶BG (+O⁶BG) for 1 h prior to TMZ (1a) addition. (FIG. 2H) Short-term viability assay curves for KL-50 (4a) in HCT116 MLH1-/- cells or HCT116 cells complemented with chromosome 3 carrying wildtype MLH1 (+Chr3) pre-treated with 0.01% DMSO control or 10 µM O⁶BG (+O⁶BG) for 1 h prior to KL-50 (4a) addition. For FIGS. 2B-2H, points, mean; error bars, SD; n≥3 technical replicates.

FIGS. 3A-3F illustrate that unrepaired primary KL-50 (4a) lesions convert to DNA ICLs in the absence of MGMT. (FIG. 3A) Scatter dot plots of the %DNA in tail upon single cell alkaline gel electrophoresis performed on LN229 MGMT-/MMR+ and MGMT-/MMR- cells treated with 0.2% DMSO control, 200 µM TMZ (1a), 200 µM KL-50 (4a), or 0.1 µM MMC (MMC*) for 24 h or with 50 µM MMC (MMC**) for 2 h. After cell lysis, comet slides were irradiated with 0 or 10 Gy prior to alkaline electrophoresis. Lines, median; error bars, 95% CI; n ≥ 160. (FIG. 3B) Representative comet images from (FIG. 3A). (FIG. 3C) Scatter dot plots of the %DNA in tail upon single cell alkaline gel electrophoresis performed on LN229 MGMT-/MMR- cells treated with 0.2% DMSO control, 200 µM MTZ (12a), 200 µM TMZ (1a), or 200 µM KL-50 (4a) for 2, 8, or 24 h. After cell lysis, comet slides were irradiated with 10 Gy prior to alkaline electrophoresis. Lines, median; error bars, 95% CI; n≥230. Data from samples treated with 0 Gy are shown in FIGS. 10C and 10D. (FIG. 3D) Representative comet images from FIG. 3C. (FIG. 3E) Denaturing gel electrophoresis of genomic DNA isolated from LN229 MGMT-/MMR+ cells treated with 0.2% DMSO control, 200 µM KL-50 (4a), 200 µM TMZ (1a), 200 µM KL-85 (4b), or 200 µM MTIC (1b) for 24 h or with 50 µM MMC or 200 µM CCNU (14) for 2 h. (FIG. 3F) Denaturing gel electrophoresis of linearized 100 ng pUC19 plasmid DNA treated in vitro with 100 µM Cisplatin (36 hours), 100 µM MMS (36 hours), 100 µM of KL-50 (4a) or 12b for 6-36 hours. For FIG. 3E and FIG. 3F, bands representing crosslinked DNA are indicated by arrows.

FIGS. 4A-4I illustrate that KL-50 (4a) activates DNA damage response pathways and cycle arrest in MGMT- cells, independent of MMR, and induces sensitivity in cells deficient in ICL or HR repair. (FIGS. 4A, 4B, and 4C) Phospho-SER139-H2AX (yH2AX) (FIG. 4A), 53BP1 (FIG. 4B), and phospo-SER33-RPA2 (pRPA) (FIG. 4C) foci formation quantified by % cells with ≥10 foci in LN229 MGMT+/-, MMR+/- cells treated with 0.1% DMSO control, 20 µM KL-50 (4a), or 20 µM TMZ (1a) for 48 h. Columns, mean; error bars, SD; n≥5 technical replicates. Additional time course data is presented in FIGS. 12B to 12D. (FIG. 4D) Representative foci images of data in (FIG. 4A) to (FIG. 4C). (FIG. 4E) Percentage of cells in G1, S, and G2 cell cycle phases after treatment as in (FIG. 4A) to (FIG. 4C), measured using integrated nuclear (Hoechst) staining intensity. Columns, mean; error bars, SD; n = 3 independent analyses. Additional time course data, cell cycle controls, and representative histograms are presented in FIGS. 13A-13B. (FIG. 4F) Change in percent cells with ≥ 1 micronuclei from baseline (DMSO control) after treatment as in (FIG. 4A) to (FIG. 4C). Columns, mean; error bars, SD; n≥15 technical replicates; **** p < 0.0001; ns, not significant. Additional validation is presented in FIGS. 15A and 15B. (FIG. 4G) Short-term viability assay curves for KL-50 (4a) in PD20 cells, deficient in FANCD2 (FANCD2-/-) or complemented with FANCD2 (+FANCD2). (FIG. 4H) Short-term viability assay curves for KL-50 (4a) in PEO4 (BRCA2+) and PEO1 (BRCA2-/-) cells pre-treated with 0.01% DMSO control or 10 µM O⁶BG (+O⁶BG) for 1 h prior to KL-50 (4a) addition. (FIG. 4I) Short-term viability assay curves for KL-50 (4a) in DLD1 BRCA2+/- and BRCA2-/- cells pre-treated with 0.01% DMSO control or 10 µM O⁶BG (+O⁶BG) for 1 h prior to KL-50 (4a) addition. For FIGS. 4G-4I, points, mean; error bars, SD; n = 3 technical replicates.

FIGS. 5A-5F illustrate that KL-50 (4a) is safe and efficacious on both MGMT-/MMR+ and MGMT-/MMR- flank tumors over a wide range of treatment regimens and conditions. (FIG. 5A) Xenograft LN229 MGMT-/MMR+ flank tumors treated with 3 weekly cycles of P.O. administration of 10% cyclodextrin control (n=7), TMZ (1a) (n=7, 5 mg/kg) or KL-50 (4a) (n=6, 5 mg/kg) on Monday, Wednesday, and Friday (individual spider plots in FIG. 16A). (FIG. 5B) Xenograft LN229 MGMT-/MMR- flank tumors treated with 3 weekly cycles of P.O. administration of 10% cyclodextrin control (n=6), TMZ (1a) (n=5, 5 mg/kg) or KL-50 (4a) (n=5, 5 mg/kg) on Monday, Wednesday, and Friday (individual spider plots in FIG. 16B). (FIG. 5C) Mean body weight of mice during LN229 flank tumor experiments. (FIG. 5D) Kaplan-Meier analysis of LN229 MGMT-/MMR- xenograft flank tumor-bearing mice to determine survival rate based on death, removal from study if mouse body weight loss exceeded 20% of initial body weight, or if tumor volume exceeded 2000 mm³. Both control and TMZ (1a) treated groups have a median OS of 10 weeks and KL-50 (4a) treated mice have median OS of greater than 15 weeks. (FIG. 5E) Xenograft LN229 MGMT-/MMR+ and LN229 MGMT-/MMR- flank tumors treated with PO administration of 10% cyclodextrin control (n=7), KL-50 (4a) (n=6, 3 cycles of 15 mg/kg on Monday, Wednesday, Friday), KL-50 (4a) (n=6, 1 cycle of 25 mg/kg Monday through Friday), or intraperitoneal (I.P.) administration of KL-50 (4a) (n=7, 3 cycles of 5 mg/kg on Monday, Wednesday, Friday) revealed equal efficacy with no observable increases in toxicity as measured by mice systemic weights (individual spider plots in FIG. S10C). (FIG. 5F) Xenograft LN229 MGMT-/MMR+ and LN229 MGMT-/MSH6- flank tumors with a larger average starting tumor size of ~400 mm³ and ~350 mm³ respectively, treated with 3 weekly cycles of P.O administration of 10% cyclodextrin (n=4) or KL-50 (4a) (n=4, 3 cycles of 25 mg/kg on Monday, Wednesday, and Friday). The study period was limited by control groups which had to be euthanized for exceeding the ethical maximum allowed tumor size, thus ending the study. In all panels, points, mean; error bars, SEM; *, P<0.05; **, P<0.01; ***, P<0.001; ****, P<0.0001; ns, not significant.

FIGS. 6A-6C illustrate that KL-50 (4a) is efficacious in an LN229 MGMT-/MMR-intracranial model and is well tolerated with limited myelosuppression at supratherapeutic doses. (FIG. 6A) Mean tumor size as measured by bioluminescent imaging (BLI) as relative light units (RLU; photons/sec) with SEM of xenograft LN229 MGMT-/MMR- intracranial tumors treated with 3 weekly cycles of P.O administration with 10% cyclodextrin control (n=10), TMZ (1a) (n=11, 25 mg/kg) or KL-50 (4a) (n=11, 25 mg/kg) on Monday, Wednesday, and Friday (individual spider plots in FIG. 16E). (FIG. 6B) Mean body change with SEM of mice during maximum tolerated dose experiment in non-tumor bearing mice. (FIG. 6C) Complete blood counts for mice pre-treatment and 7 days post-treatment with escalations of single dose KL-50 (4a) delivered PO. WBC lower limit of normal (LLN): 2.2 K/µL; Neutrophils LLN: 0.42 K/µL; Lymphocyte LLN: 1.7 K/µL; RBC LLN: 3.47 M/µL; Platelet LLN: 155 K/µL. *, P<0.05; ****, P<0.0001.

FIGS. 7A-7C illustrate literature precedent for the hydrolysis of various 2-haloethylguanosine lesions. (FIG. 7A) Kinetics of the hydrolysis of O6-(2-fluoroethylguanosine) (S1) at pH 7.4 and 37° C. (FIG. 7B) Kinetics of the hydrolysis of O6-(2-chloroethylguanosine) (S4) at pH 7.4 and 37° C. as reported by Parker et al. (39). (FIG. 7C) Failed hydrolysis of N7-(2-fluoroethyl)guanosine (S5) with “extensive incubation of [S5] at 37° [C] in neutral aqueous solution”.

FIGS. 8A-8K show additional analysis of TMZ (1a) derivatives in MGMT+/-, MMR+/-cell models. (FIG. 8A) Western blotting performed in LN229 MGMT-/MMR+ parental line, and cells complemented with wildtype MGMT (MGMT+/MMR+) and/or stable expression of MSH2 shRNA (MGMT+/MMR- and MGMT-/MMR-). MSH6 expression is reduced in these lines due to destabilization in the setting of loss of its heterodimeric partner MSH2. MLH1 expression is not affected by MSH2 knockdown. Vinculin serves as loading control. (FIGS. 8B, 8C, 8D, 8E, 8F, and 8G) Short-term viability assay curves for compounds 9, 10, 11, 12b, 13, and 12a in LN229 MGMT+/-, MMR+/- cells. (FIG. 8H) Clonogenic survival curves for lomustine (14) in LN229 MGMT+/-, MMR+/- cells. (FIG. 8I) Western blotting in HCT116 and DLD1 cells. HCT116 MLH1-/- and +Chr3 lines demonstrate re-expression of MLH1 and similar levels of MGMT and other MMR proteins. DLD1 BRCA2+/- and BRCA2-/- cells have known loss of MSH6 but comparable levels of MGMT and other MMR protein expression. GAPDH serves as loading control. (FIG. 8J) Western blotting performed in HCT116 MLH1-/- and +Chr3 and DLD1 BRCA2+/- and BRCA2-/- cells after exposure to 0.01% DMSO or 10 ܏M O6BG for 24 h, demonstrating O6BG-induced MGMT depletion. Vinculin serves as loading control. (FIG. 8K) Short-term cell viability curves for KL-50 (4a) and TMZ (1a) in BJ fibroblast cells. For FIGS. 8B-8H and 8K, points, mean; error bars, SD; n = 3 technical replicates.

FIGS. 9A-9J illustrate that KL-50 (4a) is effective in TMZ (1a)-resistant cells lacking other MMR proteins. (FIG. 9A) Western blotting performed in LN229 MGMT+/- cells with stable expression of shRNA targeting MSH6, MLH1, PMS2, or MSH3 to confirm depletion of the shRNA targets. In shMSH6 cells, there is reduced expression of MSH2, and in shMLH1 cells, there is loss of PMS2, due to destabilization in the setting of loss of their heterodimeric partners. GAPDH serves as loading control. (FIG. 9B) (Table S1.) IC50 values derived from short-term viability assays in LN229 MGMT+/- cells lines, +/-shRNA, treated with TMZ (1a) or KL-50 (4a). aMGMT TI (therapeutic index) = IC50 (MGMT+/MMR+) divided by IC50 (MGMT-/MMR+). bMMR RI (resistance index) = IC50 (MGMT-/MMR-) divided by IC50 (MGMT-/MMR+). (FIG. 9C) Short-term viability assay curves for TMZ (1a) in LN229 MGMT+/-, MMR+/shMSH6 cells. (FIG. 9D) Short-term viability assay curves for KL-50 (4a) in LN229 MGMT+/-, MMR+/shMSH6 cells. (FIG. 9E) Short-term viability assay curves for TMZ (1a) in LN229 MGMT+/-, MMR+/shMLH1 cells. (FIG. 9F) Short-term viability assay curves for KL-50 (4a) in LN229 MGMT+/-, MMR+/shMLH1 cells. (FIG. 9G) Short-term viability assay curves for TMZ (1a) in LN229 MGMT+/-, MMR+/shPMS2 cells. (FIG. 9H) Short-term viability assay curves for KL-50 (4a) in LN229 MGMT+/-, MMR+/shPMS2 cells.

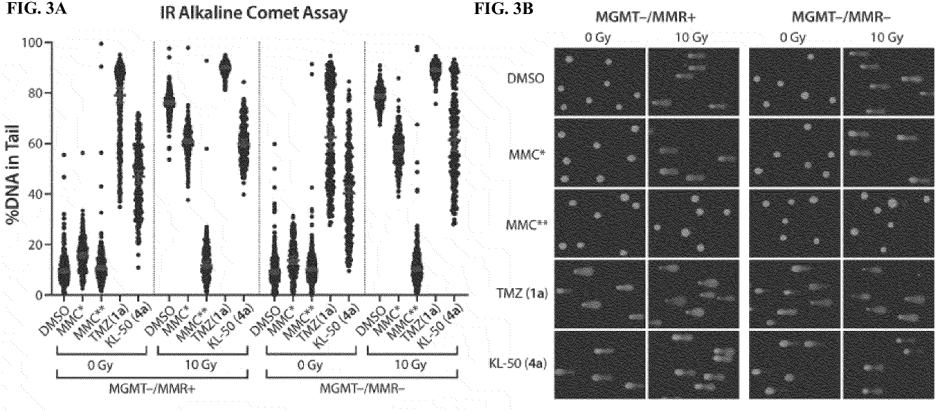

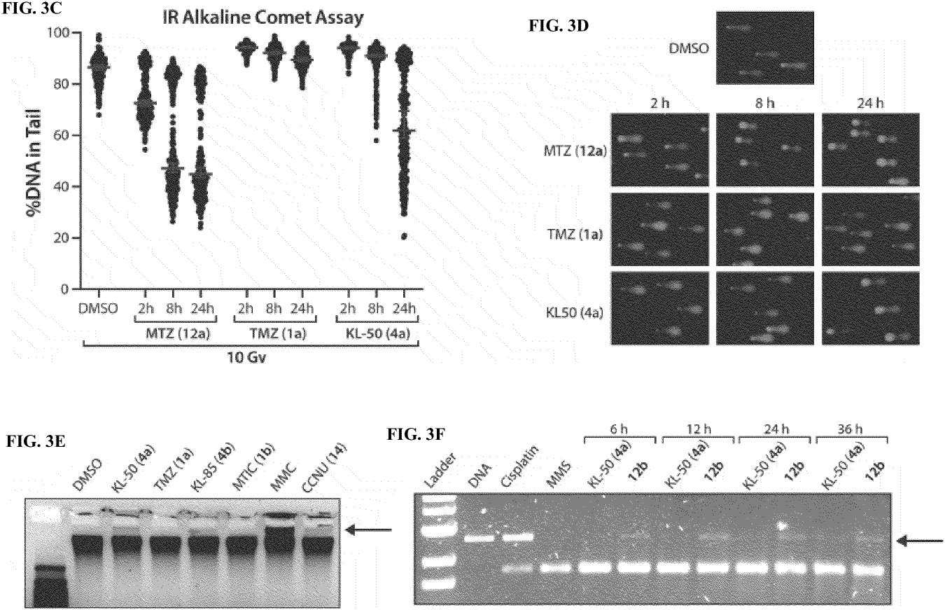

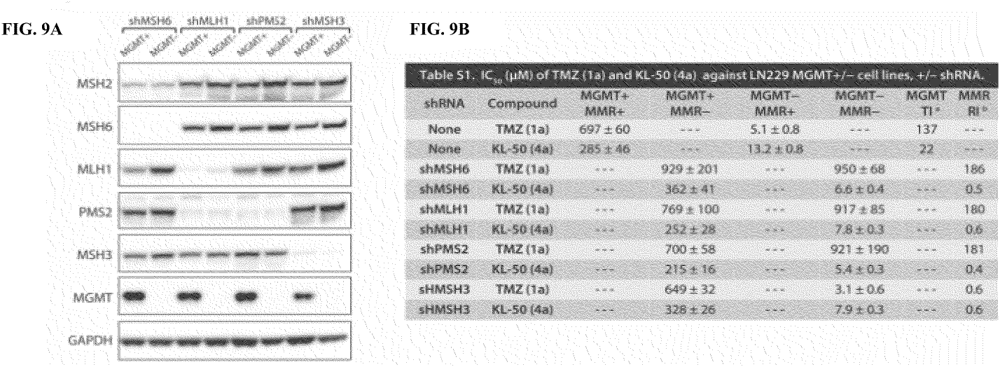

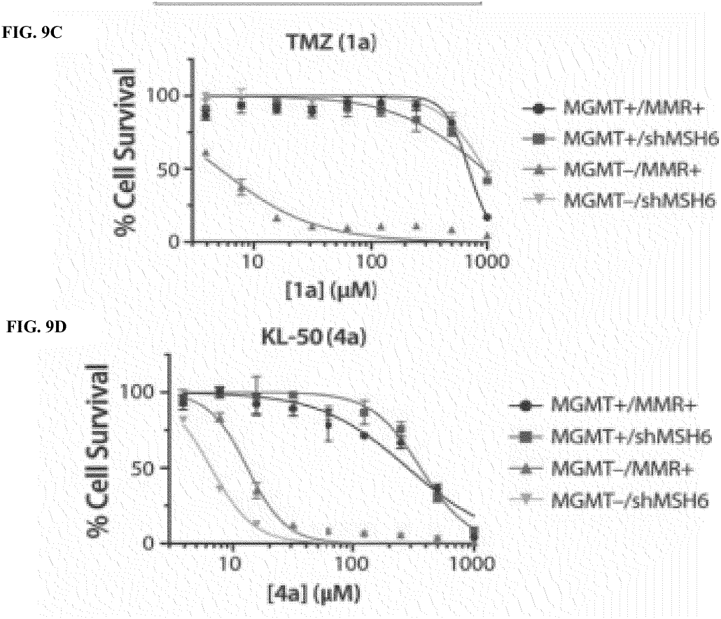

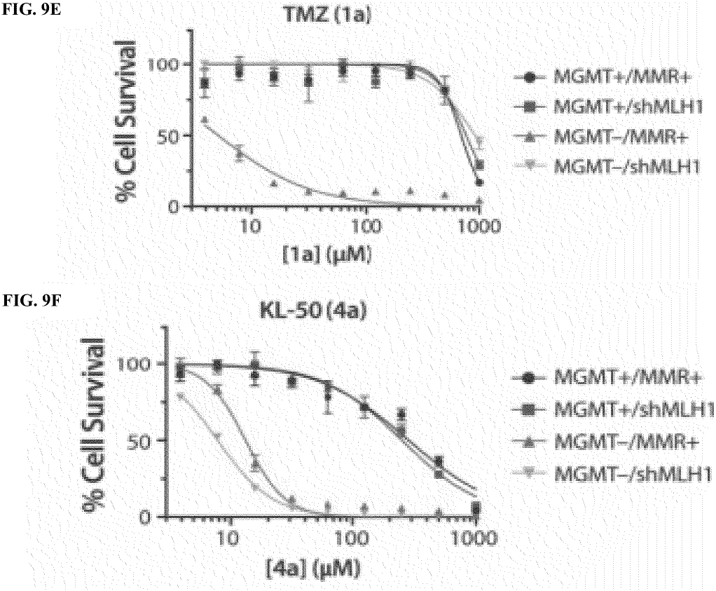

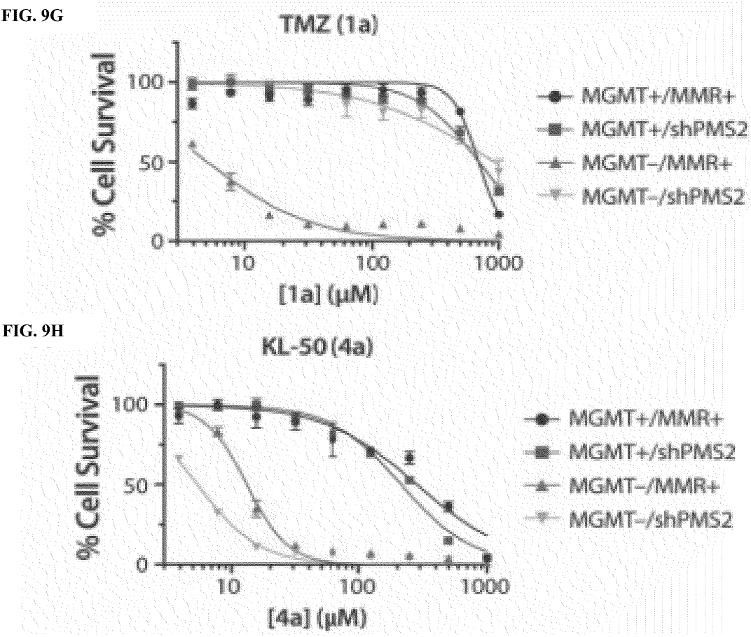

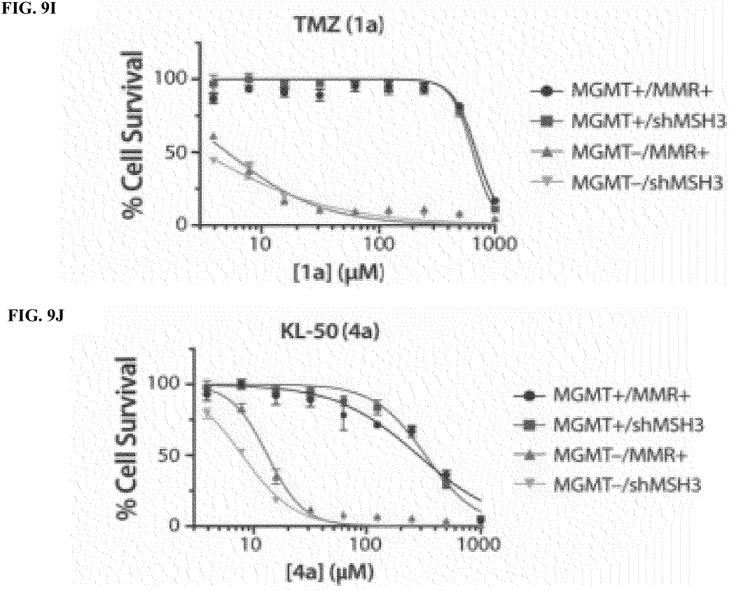

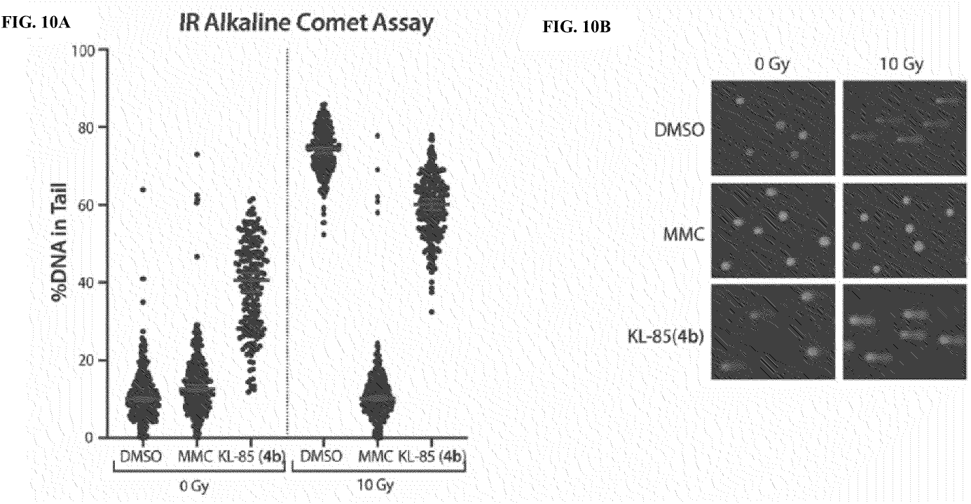

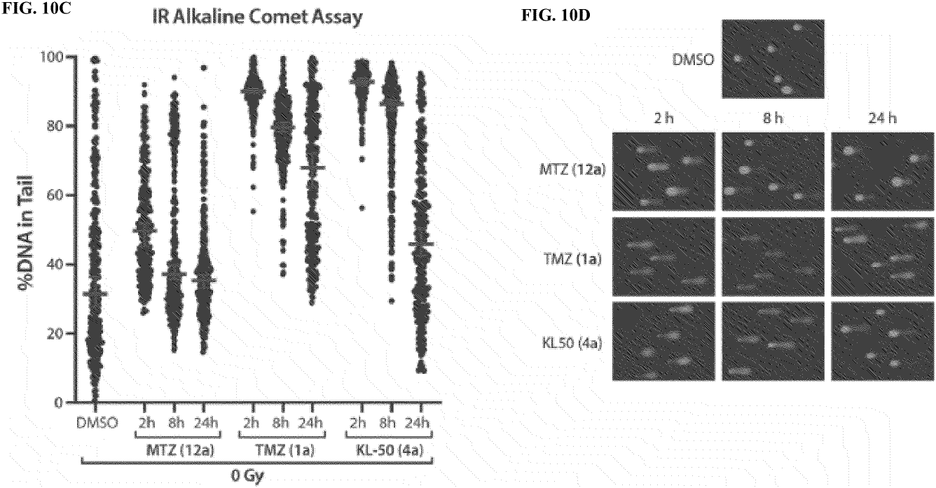

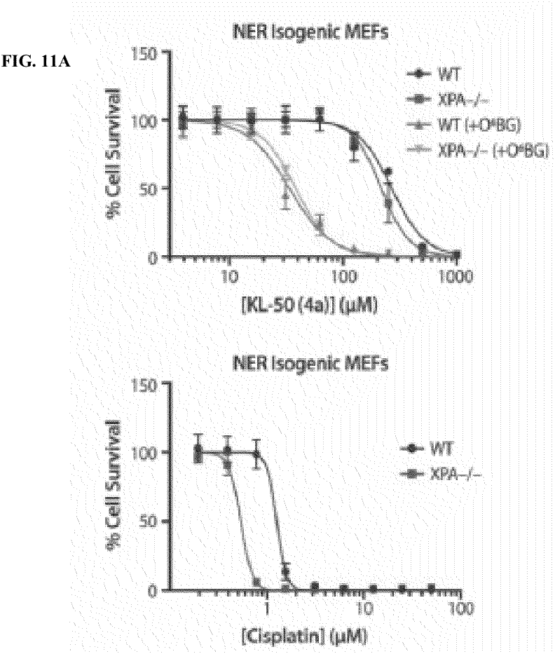

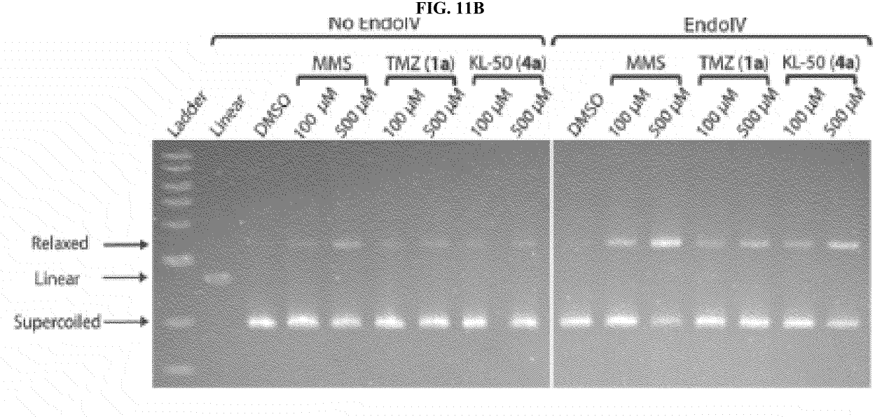

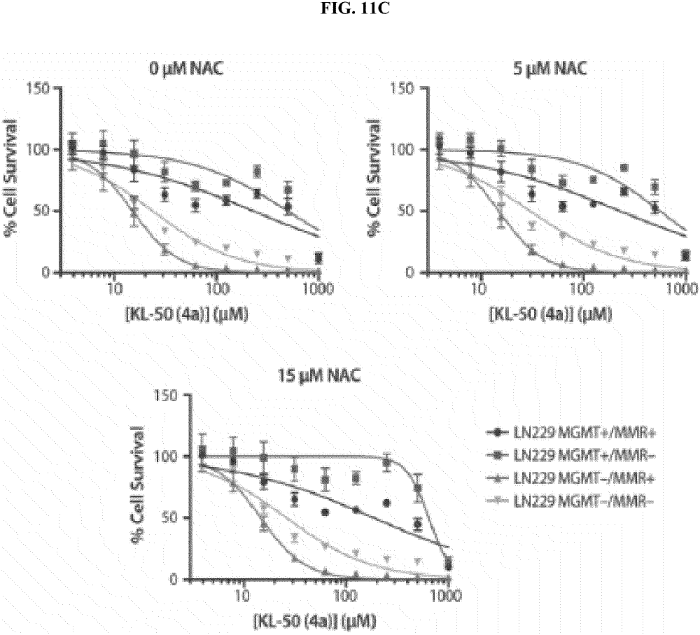

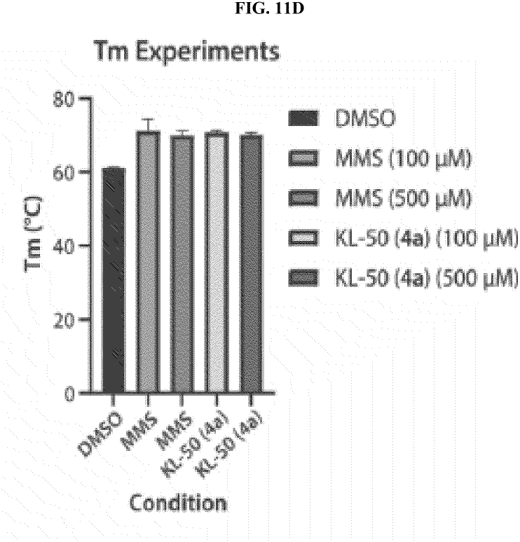

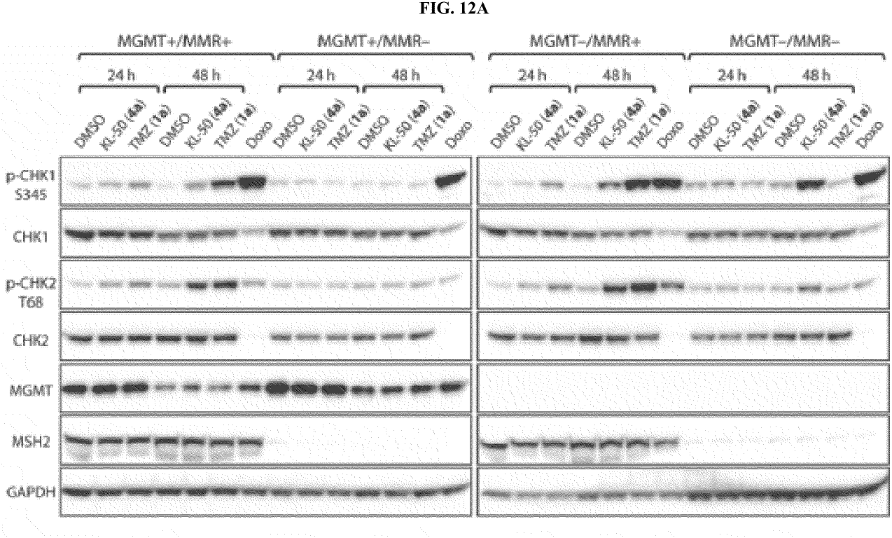

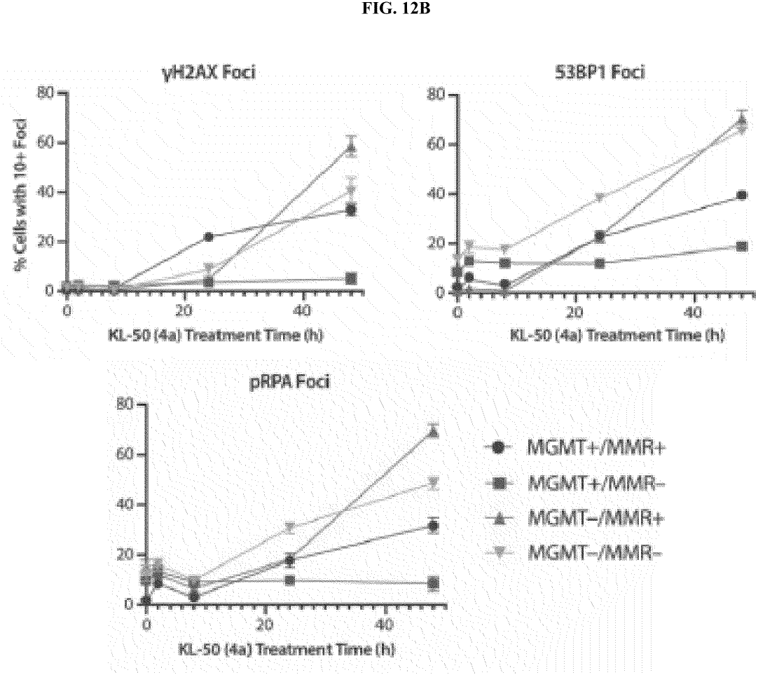

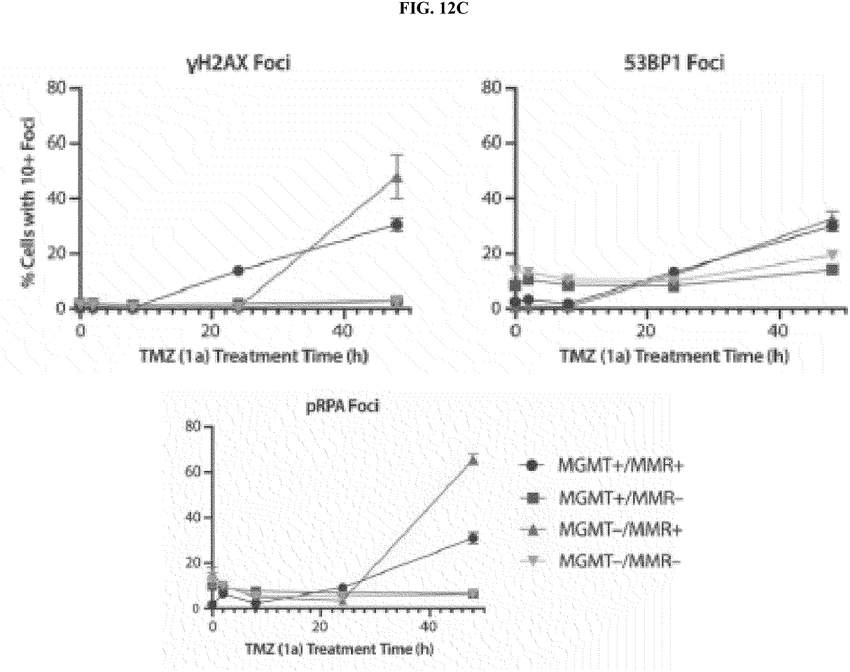

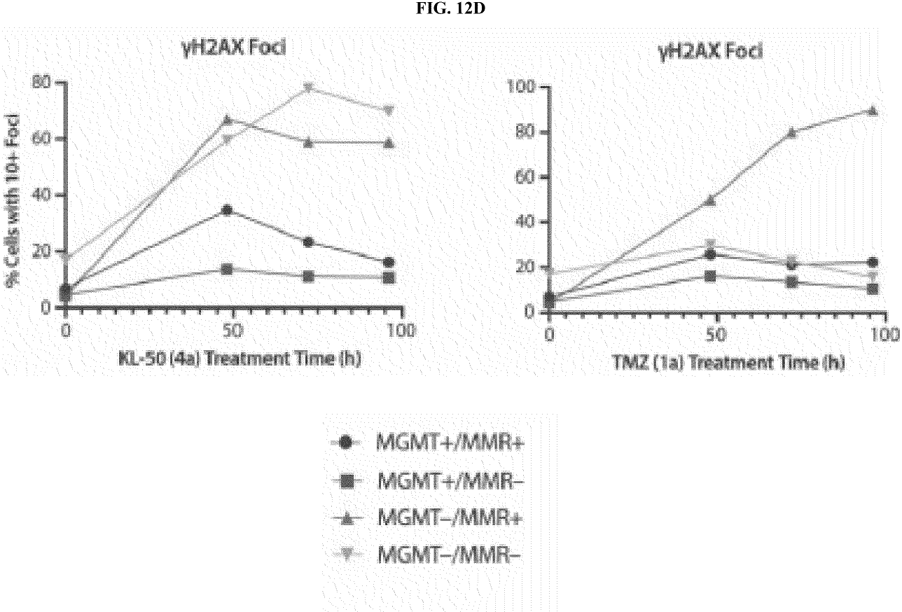

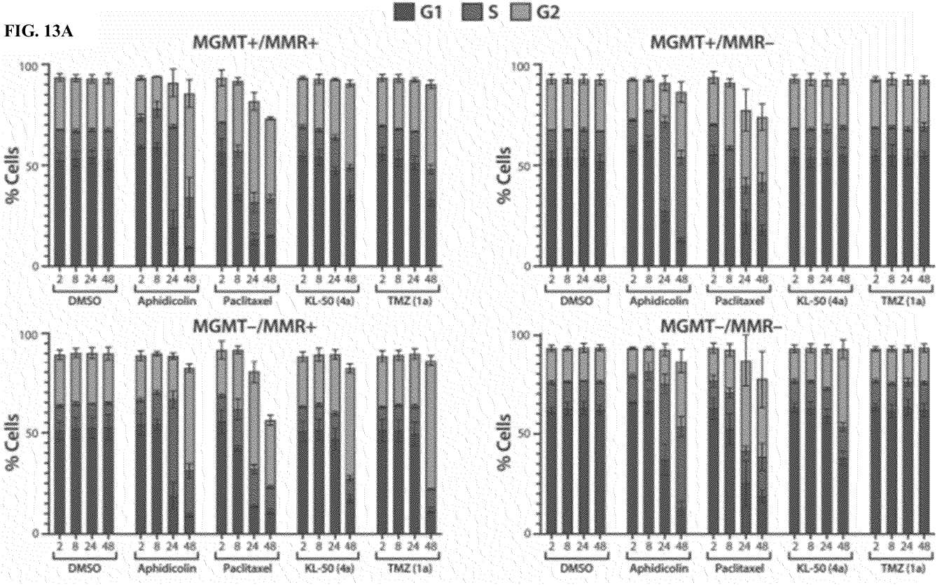

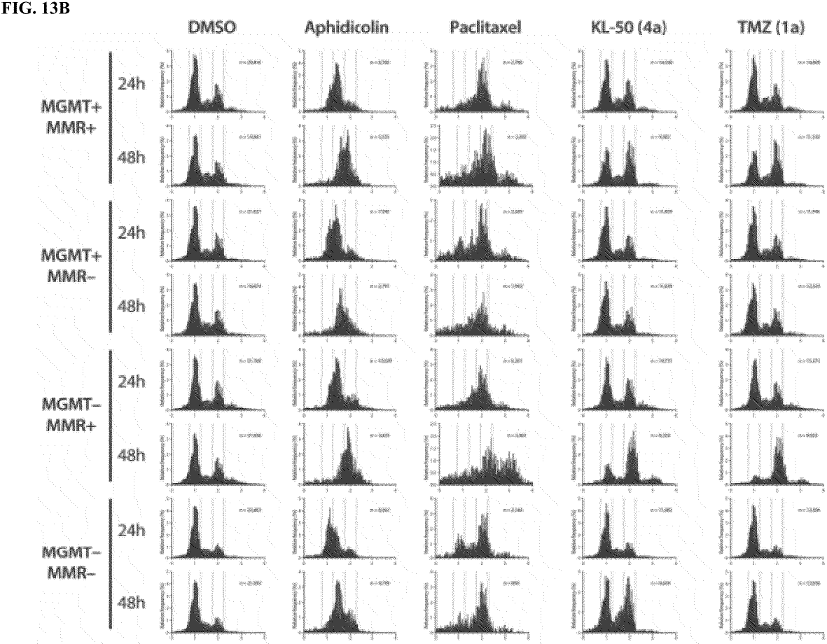

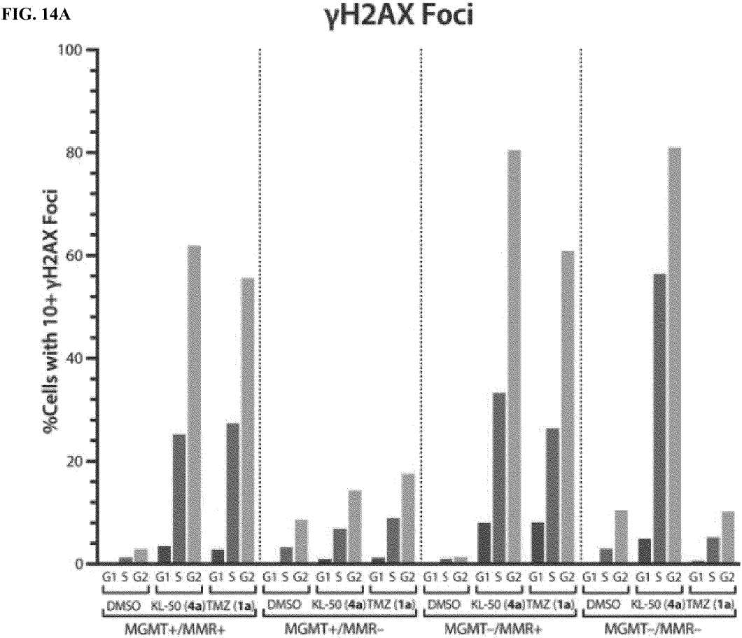

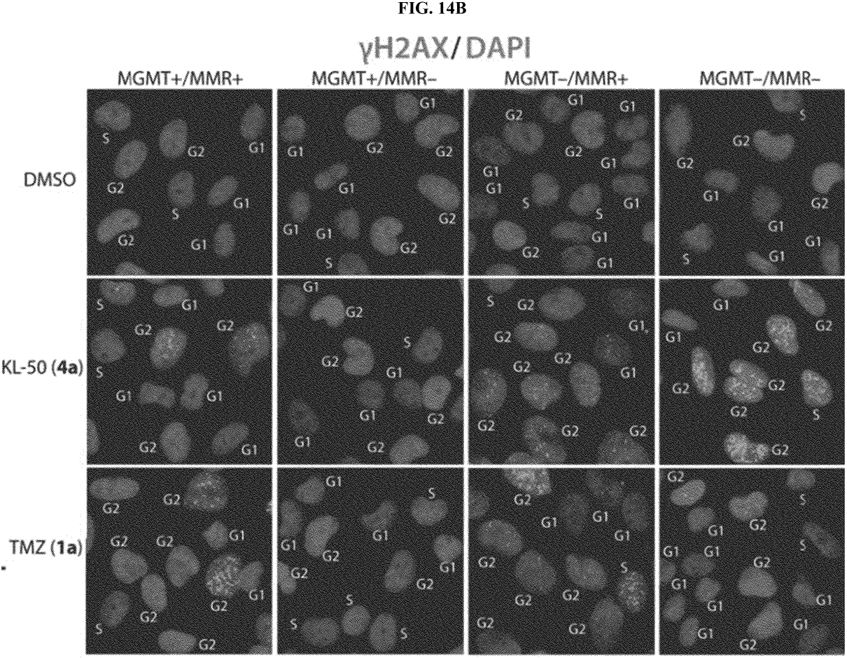

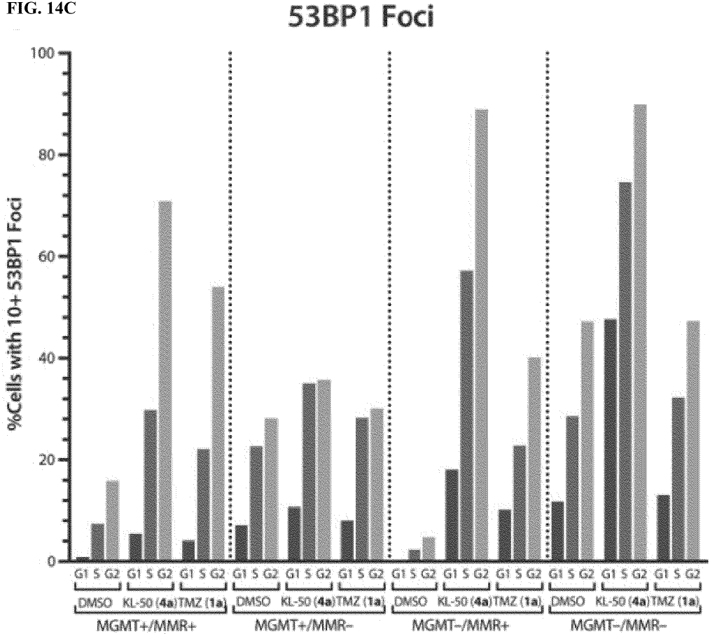

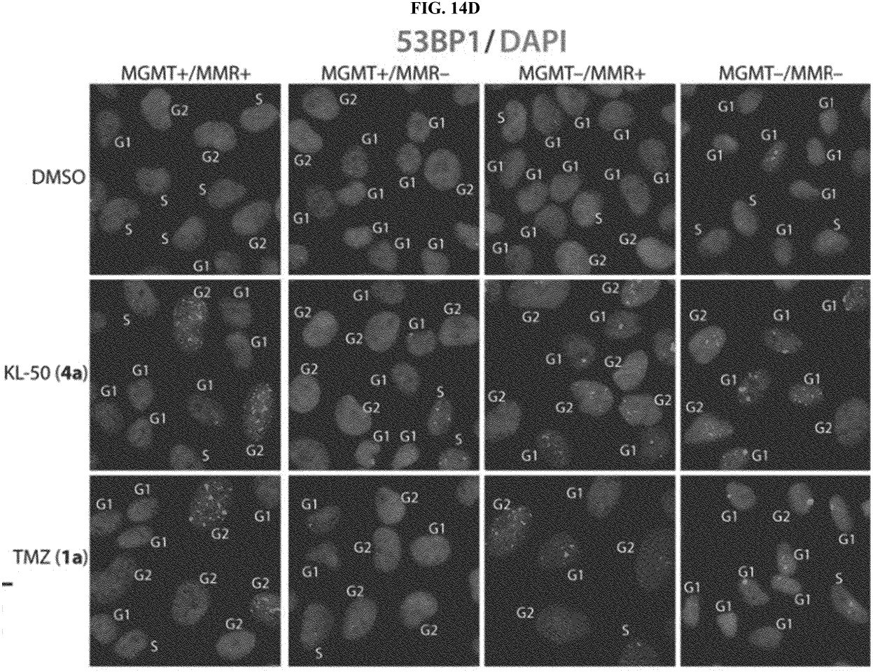

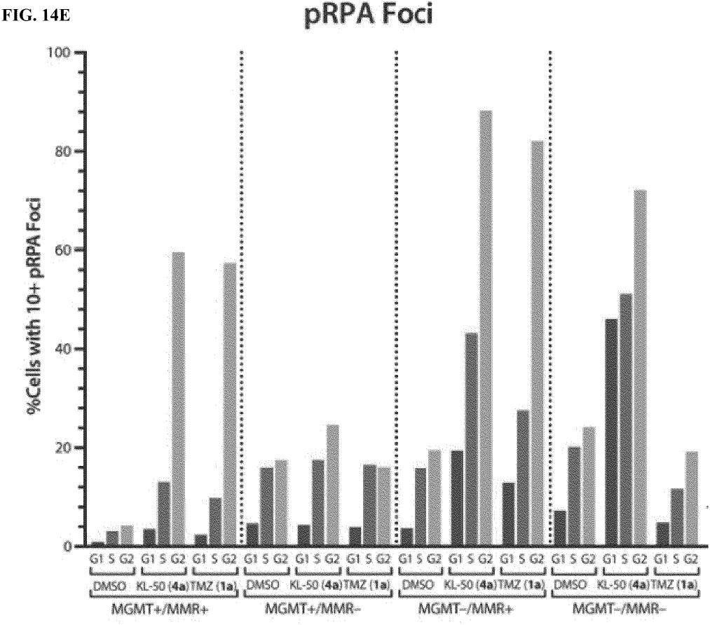

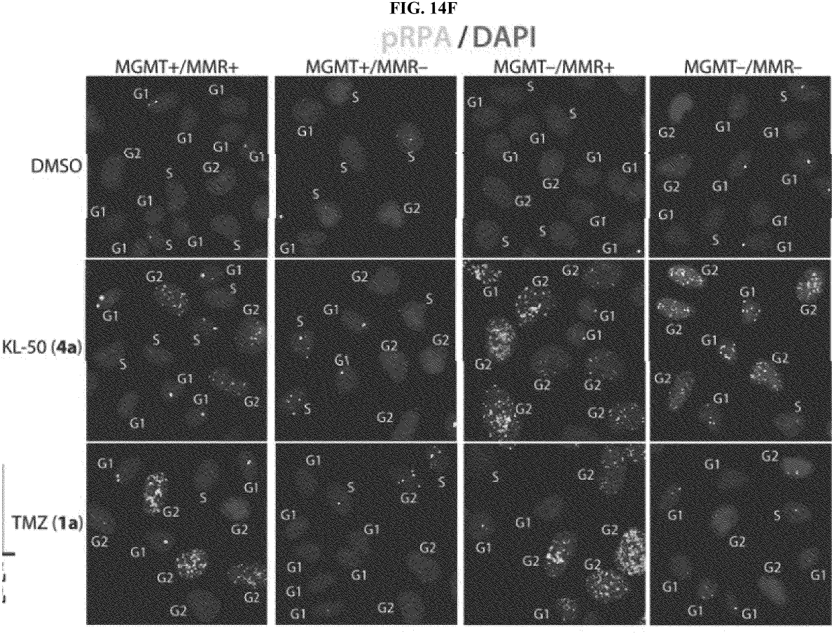

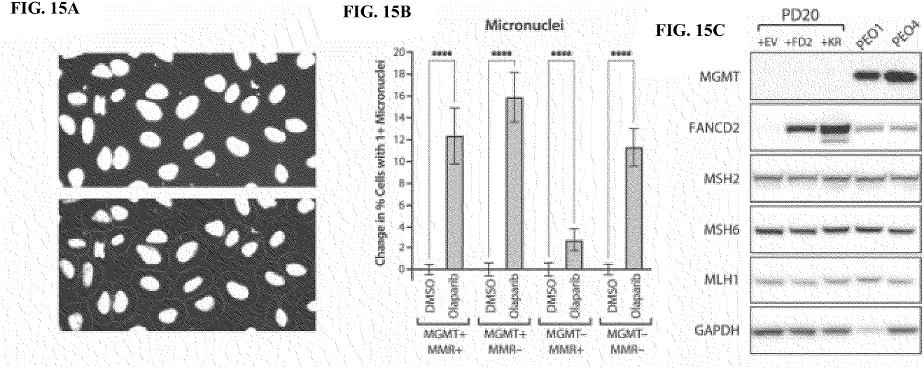

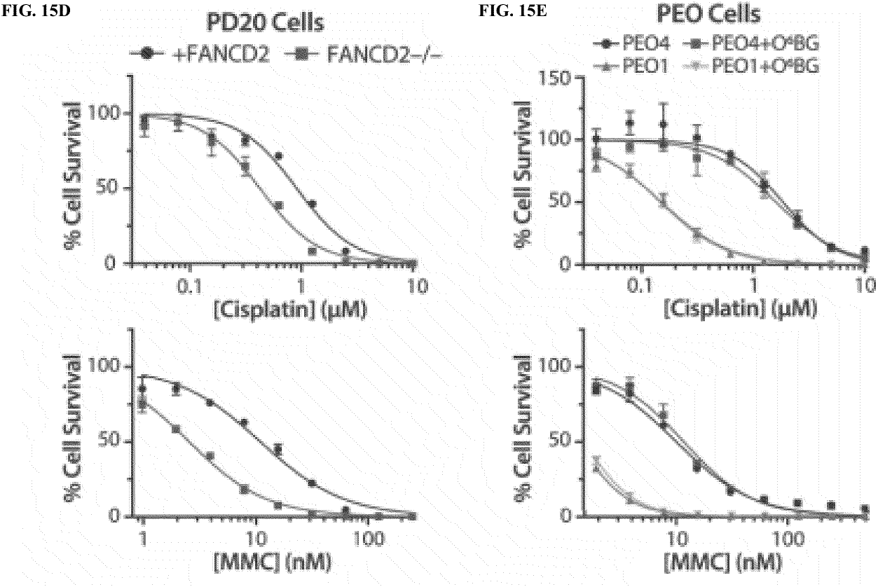

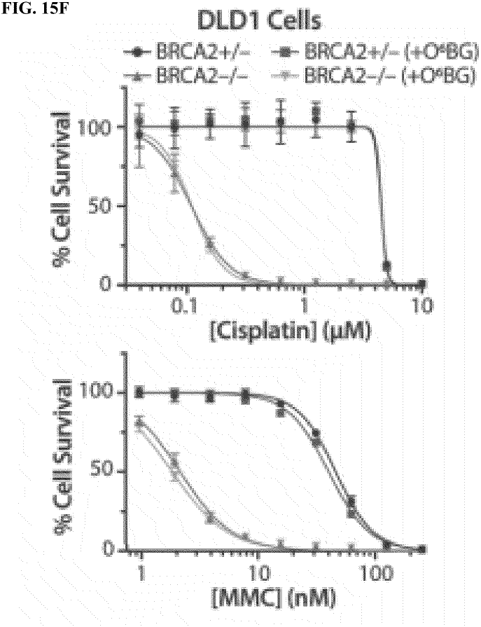

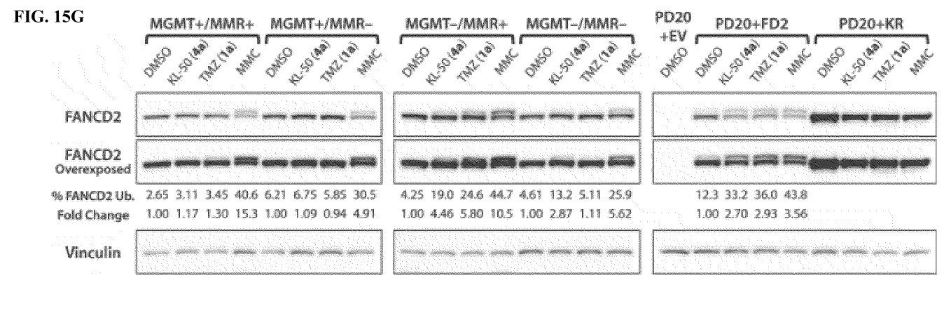

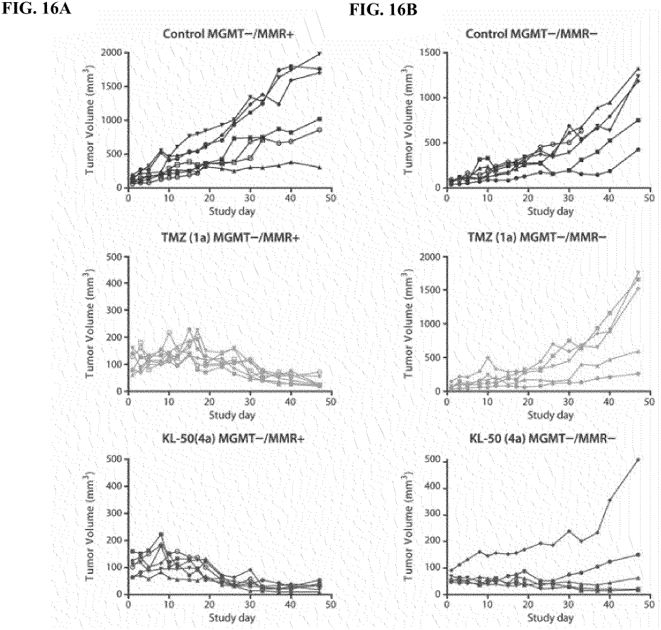

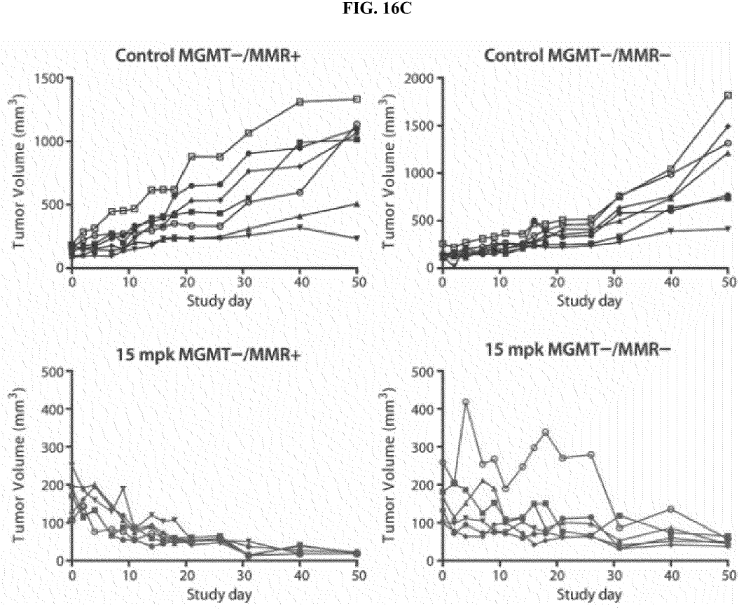

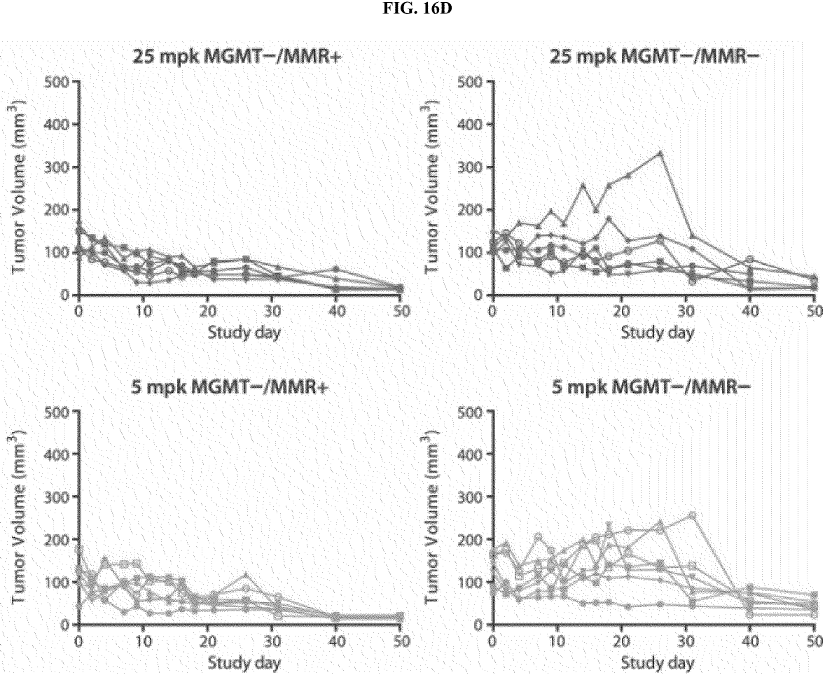

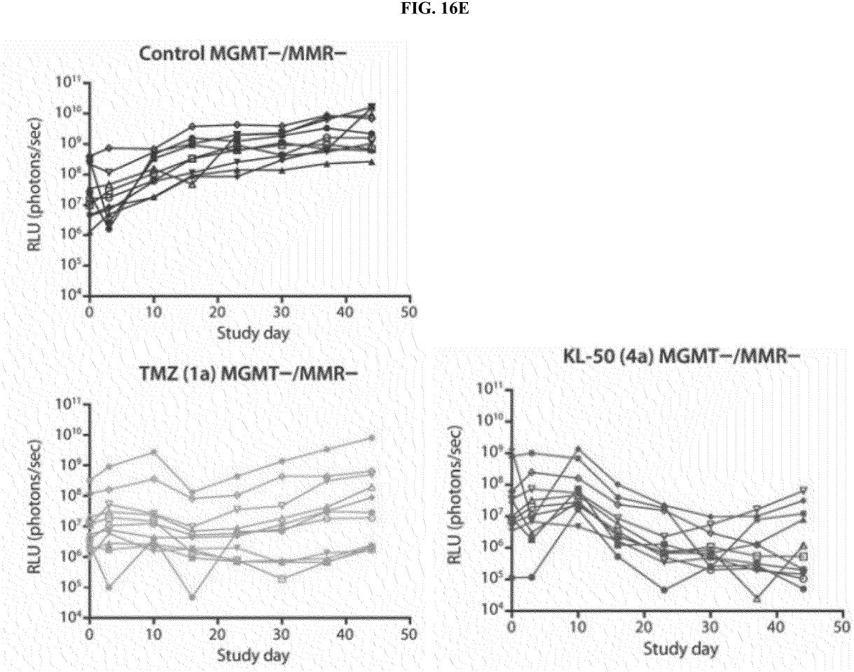

(FIG. 9I) Short-term viability assay curves for TMZ (1a) in LN229 MGMT+/-, MMR+/shMSH3 cells. (FIG. 9J) Short-term viability assay curves for KL-50 (4a) in LN229 MGMT+/-, MMR+/shMSH3 cells. For FIGS. 9C-9J, points, mean; error bars, SD; n = 3 technical replicates. FIGS. 10A-10D show supplementary IR alkaline comet assay data. (FIG. 10A) Scatter dot plots of the %DNA in tail upon single cell alkaline gel electrophoresis performed on LN229 MGMT-/MMR+ cells treated with 0.1% DMSO control or 200 µM KL-85 (4b) for 24 h or with 50 µM MMC for 2 h. After cell lysis, comet slides were irradiated with 0 or 10 Gy prior to alkaline electrophoresis. Lines, median; error bars, 95% CI; n≥160. (FIG. 10B) Representative comet images from (FIG. 10A). (FIG. 10C) Scatter dot plots of the %DNA in tail upon single cell alkaline gel electrophoresis performed on LN229 MGMT-/MMR- cells treated with 0.2% DMSO control, 200 µM MTZ (12a), 200 µM TMZ (1a), or 200 µM KL-50 (4a) for 2, 8, or 24 h. Corresponding samples treated with 10 Gy IR are shown in FIG. 3C. Lines, median; error bars, 95% CI; n≥230. (FIG. 10D) Representative comet images from (FIG. 10C).

FIGS. 11A-11D illustrate that NER, BER, ROS, and altered DNA melting point do not play a major role in the mechanism of KL-50 (4a), according to various embodiments. (FIG. 11A) Short-term cell viability assays in both WT and XPA-deficient MEFs demonstrating the absence of additional sensitivity to KL-50 (4a) in NER compromised XPA deficient cells ±MGMT depletion with O6BG, in contrast to cisplatin as positive control. (FIG. 11B) EndoIV depurination assay utilizing supercoiled pUC19 plasmid DNA assessing both spontaneous and enzymatically catalyzed SSB formation resulting from depurination post-treatment, demonstrating comparable levels of depurination and SSB formation by KL-50 (4a) and TMZ (1a). (FIG. 11C) Short-term cell viability assays in LN229 MGMT+/-, MMR+/- isogenic lines pre-treated with increasing concentrations of the ROS scavenger NAC did not result in rescue of KL-50 (4a) toxicity. (FIG. 11D) Melting temperature experiments in linearized pUC19 plasmid DNA treated with 100 or 500 µM of MMS or KL-50 (4a) for 3 h resulted in comparable changes in measured DNA melting temperature. Columns, mean; error bars, SD; n = 2 independent analyses. For (FIG. 11A) and (FIG. 11C), points, mean; error bars, SD; n = 3 technical replicates.

FIGS. 12A-12D show that KL-50 (4a) induces activation of the ATR-CHK1 and ATM-CHK2 signaling axes and delayed DNA repair foci formation in MGMT-deficient cells, independent of MMR status. (FIG. 12A) Western blotting performed in LN229 MGMT+/-, MMR+/- cells following treatment with 20 µM KL-50 (4a) or TMZ (1a) for 24 or 48 h. Treatment with 1 µM doxorubicin for 24 h (Doxo) served as a positive control for p-CHK1 activation. (FIG. 12B and FIG. 12C) Phospho-SER139-H2AX (yH2AX), 53BP1, and phospho-SER33-RPA2 (pRPA) foci levels over time following treatment with KL-50 (4a; 20 µM) (FIG. 12B) or TMZ (1a; 20 µM) (FIG. 12C) for 0, 2, 8, 24, or 48 h in LN229 MGMT+/-, MMR+/-cells. Points, mean % cells with ≥10 foci; error bars, SD; n≥5 technical replicates. (FIG. 12D) Extended time course of yH2AX foci levels following treatment with KL-50 (4a; 20 µM) or TMZ (1a; 20 µM) for 0, 48, 72, or 96 h in LN229 MGMT+/-, MMR+/- cells. Points, % cells with ≥10 foci, n≥250 cells per condition.

FIGS. 13A-13B show supplementary cell cycle analysis data. (FIG. 13A) Time course analysis of cell cycle distribution measured using integrated nuclear (Hoechst) staining intensity after treatment of LN229 MGMT+/-, MMR+/- cells with KL-50 (4a; 20 µM) or TMZ (1a; 20 µM) for 2, 8, 24, or 48 h. DMSO (0.1%) serves as negative control and aphidicolin (10 µM) and paclitaxel (1 µM) serve as positive controls for S-phase and G2-phase arrest, respectively. Columns, mean; error bars, SD; n = 3 independent analyses. (FIG. 13B) Representative histograms showing DNA content distribution from 24 h and 48 h treatment conditions as quantified in (FIG. 12A).

FIGS. 14A-14E illustrate that KL-50 (4a) induces DDR foci formation primarily in S and G2 cell cycle phases, and to lesser extent, in MGMT- G1 phase cells. (FIGS. 14A and 14B) Phospho-SER139-H2AX (yH2AX) foci levels in LN229 MGMT+/-, MMR+/- cells in G1, S, and G2 cell cycle phases after treatment with 0.1% DMSO control, KL-50 (4a; 20 µM) or TMZ (1a; 20 µM) for 48 h. Representative foci images with nuclei labeled as G1, S, or G2 phase cells based on Hoechst staining intensity are shown on the right. (FIGS. 14C and 14D) 53BP1 foci levels and representative foci images in cells treated as in (FIGS. 14A and 14B). (FIGS. 14E and 14F) Phospho-SER33-RPA2 (pRPA) foci levels and representative foci images in cells treated as in (FIGS. 14A and 14B). For (FIGS. 14A and 14B), (FIGS. 14C and 14D), and (FIGS. 14E and 14F), points, % cells with ≥10 foci; n≥500 cells per condition and cell cycle phase.

FIGS. 15A-15G show the validation of micronuclei analysis, ICL sensitivity in FANCD2-/- and BRCA2-/- cell models, and demonstration of FANCD2 ubiquitination induced by KL-50 (4a). (FIG. 15A) Representative images of micronuclei identification. (FIG. 15B) Validation of micronuclei identification using olaparib as positive control. Change in percent cells with ≥1 micronuclei from baseline (DMSO control) after treatment with olaparib (10 µM) for 48 h in LN229 MGMT+/-, MMR+/- cells. Columns, mean; error bars, SD; n ≥15 technical replicates; **** p <0.0001. (FIG. 15C) Western blotting performed in PD20 cells complemented with empty vector (EV), wildtype FANCD2 (WT), or ubiquitination-mutant FANCD2 (KR), demonstrating loss of MGMT in PD20 cells and comparable expression of MMR proteins. Western blotting in PEO1 BRCA2-/- and PEO4 BRCA2+ cells demonstrates intact expression of MGMT and MMR proteins. (FIG. 15D) Short-term viability assay curves for cisplatin and mitomycin (MMC) in PD20 cells, deficient in FANCD2 (FANCD2-/-) or complemented with FANCD2 (+FANCD2), demonstrating hypersensitivity to crosslinking agents in FANCD2-/-cells. (FIG. 15E) Short-term viability assay curves for cisplatin and MMC in PEO4 (BRCA2+) and PEO1 (BRCA2-/-) cells pre-treated with 0.01% DMSO control or 10 µM O⁶BG (+O⁶BG) for 1 h prior to cisplatin or MMC addition, demonstrating hypersensitivity of PEO4 BRCA2-/-cells to crosslinking agents independent of MGMT depletion. (FIG. 15F) Short-term viability assay curves for cisplatin and MMC in DLD1 BRCA2+/- and BRCA2-/- cells pre-treated with 0.01% DMSO control or 10 µM O⁶BG (+O⁶BG) for 1 h prior to cisplatin or MMC addition, demonstrating hypersensitivity of DLD1 BRCA2-/- cells to crosslinking agents independent of MGMT depletion. (FIG. 15G) Western blot analysis of FANCD2 ubiquitination in LN229 MGMT+/-, MMR+/- cells and PD20 FANCD2-deficient cells, complemented with empty vector (FANCD2+EV), wildtype FANCD2 (PD20+FD2) or ubiquitination-mutant FANCD2 (PD20+KR). The % FANCD2 ubiquitination (% FANCD2 Ub.) is quantified as the background-corrected integrated band intensity of the upper band divided by the sum of the background-corrected integrated band intensities of the upper and lower bands. The fold change in % FANCD2 ubiquitination is presented for each cell line relative to DMSO-treated cells. Vinculin serves as loading control. For (FIG. 15D), (FIG. 15E), and (FIG. 15F), points, mean; error bars, SD; n = 3 technical replicates.

FIGS. 16A-16E show spider plots tracking individual mouse tumor response to treatment. (FIG. 16A) Spider plots tracking LN229 MGMT-/MMR+ flank tumor volume of each mouse in response to treatment with P.O. 10% cyclodextrin vehicle control, TMZ (1a, 5 mg/kg MWF × 3 weeks ), or KL-50 (4a, 5 mg/kg MWF × 3 weeks). (FIG. 16B) Spider plots tracking LN229 MGMT-/MMR- flank tumor volume of each mouse in response to treatment with PO 10% cyclodextrin vehicle control, TMZ (1a, 5 mg/kg MWF × 3 weeks ), or KL-50 (4a, 5 mg/kg MWF × 3 weeks). (FIG. 16C and FIG. 16D) Spider plots tracking LN229 MGMT-/MMR+ and LN229 MGMT-/MMR- flank tumor volume in response to treatment with P.O 10% cyclodextrin control, P.O KL-50 (4a, 15 mg/kg MWF × 3 weeks), P.O KL-50 (4a, 25 mg/kg M-F × 1 week), or I.P. KL-50 (4a, 5 mg/kg MWF × 3 weeks). (FIG. 16E) Spider plots tracking LN229 MGMT-/MMR- intracranial tumor size as measured by relative light units (photons/sec) in response to P.O treatment with 10% cyclodextrin vehicle control, TMZ (1a, 25 mg/kg M-F × 1 week), or KL-50 (4a, 25 mg/kg M-F × 1 week).

### DETAILED DESCRIPTION

In accordance with the present disclosure, there is provided a method of treating, ameliorating, and/or preventing an MGMT deficient cancer in a patient in need of such treatment. In certain embodiments, the method comprises administering to said patient a therapeutically-effective amount of a compound of formula (I) or a pharmaceutically-acceptable salt thereof:

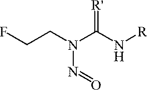

wherein R′ is selected from O and NH; and R is selected from H, NO₂, CH₂CH₂Cl, CH₂CH₂F, cyclohexyl, 4-methylcyclohexyl, —C(H)(CH₃)—P(═O)(OCH₂CH₃)₂,

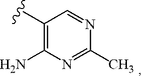

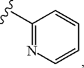

and

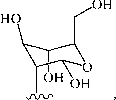

as well as pharmaceutical compositions comprising a compound of formula (I) or a pharmaceutically-acceptable salt thereof and certain compound of formula (I). In some embodiments, the pharmaceutical compositions comprise an amount of a compound of formula (I) or a pharmaceutically-acceptable salt thereof that is effective to treat the MGMT deficient cancer.

Compounds disclosed herein are potent anti-cancer agents against MGMT deficient cancers regardless of MMR status and particularly against cancers that are MGMT and MMR deficient.

Also provided herein is a method of treating cancer that is MGMT and MMR deficient in a patient in need of such treatment comprising administration of a therapeutically-effective amount of a compound of formula (I) or a pharmaceutically-acceptable salt thereof to said patient. In one embodiment, the cancer is selected from urothelial cancer, breast invasive carcinoma, colon adenocarcinoma, head and neck tumor (SCC), lung adenocarcinoma, rectum adenocarcinoma, acute myeloid leukemia, glioblastoma multiforme, brain lower grade glioma, Hodgkin’s lymphoma, non-Hodgkin’s lymphoma, and melanoma.

In one embodiment, the cancer to be treated in the present method is glioblastoma multiforme.

### Definitions

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

As used herein, a “disease” is a state of health of a subject wherein the subject cannot maintain homeostasis, and wherein if the disease is not ameliorated then the subject’s health continues to deteriorate.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

As used herein, a “disorder” in a subject is a state of health in which the subject is able to maintain homeostasis, but in which the subject’s state of health is less favorable than it would be in the absence of the disorder. Left untreated, a disorder does not necessarily cause a further decrease in the subject’s state of health.

The term “glioma” as used herein refers to a common type of tumor originating in the brain. About 33 percent of all brain tumors are gliomas, which originate in the glial cells that surround and support neurons in the brain, including astrocytes, oligodendrocytes and ependymal cells

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaralkynyl” as used herein refers to alkynyl groups as defined herein in which a hydrogen or carbon bond of an alkynyl group is replaced with a bond to a heteroaryl group as defined herein. Representative aralkynyl groups include, but are not limited to, 2-ethynylpyridine and 2-ethynylthiophene.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl) , indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, ,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3- pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “knockdown” or “KD” as used herein refers to an experimental technique wherein the expression of one or more of an organisms genes and/or translation of the corresponding RNA is reduced.

As used herein, a “prophylactic” or “preventive” treatment is a treatment administered to a subject who does not exhibit signs of a disease or disorder or exhibits only early signs of the disease or disorder for the purpose of decreasing the risk of developing pathology associated with the disease or disorder.

As used herein, the language “pharmaceutically effective amount,” “therapeutically effective amount,” or “effective amount” refers to a non-toxic but sufficient amount of the composition used in the practice of the disclosure that is effective to treat, prevent, and/or ameliorate a disease or disorder in the body of a mammal. The desired treatment may be prophylactic and/or therapeutic. That result may be reduction and/or alleviation of the signs, symptoms, or causes of a disease or disorder, or any other desired alteration of a biological system. An appropriate therapeutic amount in any individual case may be determined by one of ordinary skill in the art using routine experimentation.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer’s solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington’s Pharmaceutical Sciences (Genaro, Ed., Mack Publishing Co., 1985, Easton, PA), which is incorporated herein by reference.

In certain embodiments, the pharmaceutically-acceptable carrier is a material for admixture with a pharmaceutical compound (e.g., a chimeric compound) for administration to a patient as described, for example, in “Ansel’s Pharmaceutical Dosage Forms and Delivery Systems”, Tenth Edition (2014).

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

The term “room temperature” as used herein refers to a temperature of about 15° C. to about 28° C.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less. The term “substantially free of” can mean having a trivial amount of, such that a composition is about 0 wt% to about 5 wt% of the material, or about 0 wt% to about 1 wt%, or about 5 wt% or less, or less than, equal to, or greater than about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less, or about 0 wt%.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

As used herein, the term “treating” means ameliorating the effects of, or delaying, halting or reversing the progress of a disease or disorder. The word encompasses reducing the severity of a symptom of a disease or disorder and/or the frequency of a symptom of a disease or disorder. In certain embodiments, the treatment or treating when used in reference to cancer refers to reducing, ameliorating or eliminating one or more symptoms or effects of the disease or condition.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, the term “comprising” is intended to mean that the compositions and methods include the recited elements, but not excluding others. “Consisting essentially of′ when used to define compositions and methods, shall mean excluding other elements of any essential significance to the composition or method. “Consisting of′ shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “loweralkyl” means a linear or branched saturated hydrocarbon of 1 to 5 carbon atoms, including methyl, ethyl, propyl, isopropyl, butyl, 2-methylpropyl, tert-butyl, and pentyl.

As used herein, “optional” or “optionally” means that the subsequently described event or circumstance may or may not occur, and that the description includes instances where said event or circumstance occurs and instances where it does not.

As used herein, the terms “individual”, “patient”, or “subject” can be an individual organism, a vertebrate, a mammal (e.g., a bovine, a canine, a feline, or an equine), or a human. In a preferred embodiment, the individual, patient, or subject is a human.

As used herein, the phrases “therapeutically effective amount” and “therapeutic level” mean a compound dose or plasma concentration in a subject, respectively, that provides the specific pharmacological effect for which the compound is administered in a subject in need of such treatment, i.e., to reduce, ameliorate, or eliminate the effects or symptoms of cancer. It is emphasized that a therapeutically effective amount or therapeutic level of a drug will not always be effective in treating the conditions/diseases described herein, even though such dosage is deemed to be a therapeutically effective amount by those of skill in the art. The therapeutically effective amount may vary based on the route of administration and dosage form, the age and weight of the subject, and/or the subject’s condition, including the type and stage of the cancer at the time that treatment commences, among other factors.

A “therapeutic response” means an improvement in at least one measure of cancer.

As used herein, the term “refractory” as to a particular treatment of a disease means that the disease is unresponsive to the treatment.

As used herein the term “MGMT deficient” (or MGMT⁻) cancers means cancers that have more than one standard deviation lower abundance of the mRNA transcript for the MGMT gene or more than one standard deviation lower abundance of the associated functional protein itself normalized to the relevant healthy control tissue. This deficiency can occur through promoter methylation, mutations in the gene, or through other methods resulting in downregulation of the gene.

As used herein, the term “MMR deficient” (or MMR⁻) cancers means cancers that have more than one standard deviation lower abundance of the mRNA transcript for any of the MMR genes (MSH2, MSH6, MLH1, MLH3, PMS2, PMS1) or more than one standard deviation lower abundance of the respective functional protein(s) normalized to the relevant healthy control tissue. Alternatively, cancers that exhibit the microsatellite instability high phenotype (MSI-H) are also considered to be MMR deficient. See, for example, Li et al. - Microsatellite instability: a review of what the oncologist should know - Cancer Cell International, Article Number 16 (2020).

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 and so forth, as well as individual numbers within that range, for example, 1, 2, 2.7, 3, 4, 5, 5.1, 5.3, 5.5, and 6. This applies regardless of the breadth of the range.

### Part A: Nitrosourea Compounds

Specific examples of compounds of formula (I) include the following:

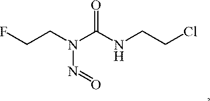

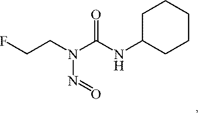

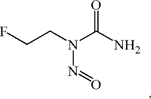

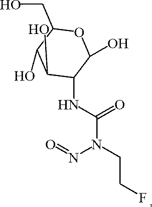

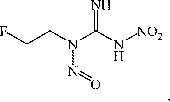

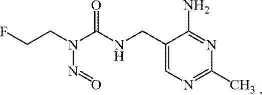

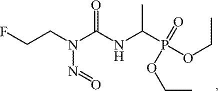

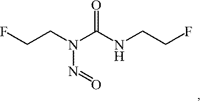

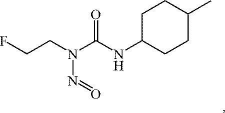

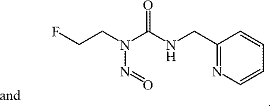

In various embodiments, compounds of formula (I) include the following:

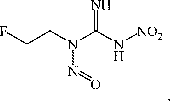

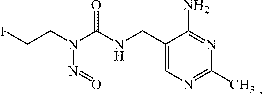

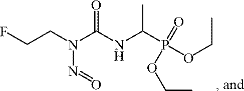

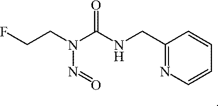

Certain of the compounds of formula (I) may be synthesized as disclosed in the literature, as follows:

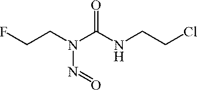 [SMILES: O=NN(CCF)C(=O)NCCCl]

Canadian Journal of Chemistry 1984, 62, 2107-12;

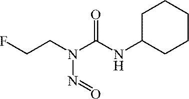 [SMILES: O=NN(CCF)C(=O)NC1CCCCC1]

Synthesis 1987, 1027-9;

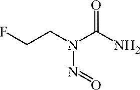

Journal of Organic Chemistry 1981, 46, 5309-21;

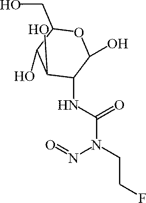 [SMILES: O=NN(CCF)C(=O)NC1C(O)OC(CO)C(O)C1O]

Bioorganic & Medicinal Chemistry Letters, 2020, 30, 127400.

Other compounds of formula (I) may be synthesized by the following general synthetic schemes: Synthetic procedure for:

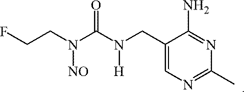

The titled compound is prepared by the following route:

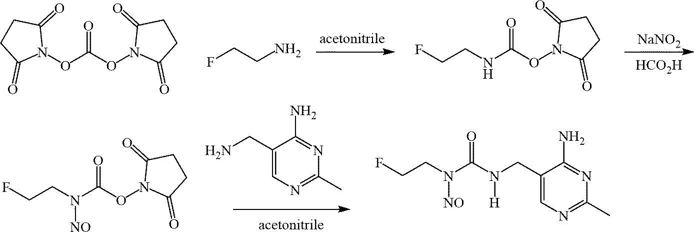

Step 1: This procedure follows that referenced in Tetrahedron Letters, 1983, 24, 4569-4572. To a solution of 2-fluoroethylamine (1.0 equiv.) in acetonitrile (0.1 M) is added a solution of N,N′-Disuccinimidyl carbonate (2 equiv.) in acetonitrile (0.1 M) at room temperature and the reaction is stirred until complete. The reaction is concentrated to dryness on a rotary evaporator and the residue is dissolved in chloroform. Insoluble unreacted N,N′-Disuccinimidyl carbonate is removed by filtration. The organic layer is washed successively with water, aqueous 1 M HCl, aqueous 4% sodium bicarbonate, aqueous saturated sodium chloride, then dried over solid anhydrous sodium sulfate. The solvent is removed by rotary evaporation to afford the titled compound

Step 2: Carbonate N-oxidation: This procedure follows that referenced in J. Med. Chem. 1980, 23, 848-851. Starting carbonate (1.0 equiv.) is dissolved in formic acid (0.75 M) and cooled to 0° C. in an ice bath. Next, NaNO2 (3.0 equiv.) is added in small portions while maintaining internal temperature below 5° C. The reaction is stirred for 30 minutes below 5° C. Next, the reaction is neutralized to pH 7 with 10% aqueous sodium hydroxide solution. The reaction is extracted with ethyl acetate. Combined organic layers are dried over anhydrous solid sodium sulfate, filtered and concentrated to dryness on a rotovap. The crude residue is purified by silica gel column chromatography eluting with an appropriate mixture of methanol and methylene chloride or chloroform and ethyl acetate to afford the titled compound.

Step 3: Displacement of N-hydroxysuccinimide: This procedure follows that referenced in Tetrahedron Letters, 1983, 24, 4569-4572. Starting N-nitrosocarbonate (1.0 equiv.) in acetonitrile (0.1 M) is added a solution of 4-Amino-2-methyl-5-pyrimidinemethanamine (1.0 equiv.) in acetonitrile (0.5 M) and the mixture is stirred at room temperature until complete. The reaction is concentrated to dryness on a rotary evaporator to afford a crude residue. This residue is purified by silica gel column chromatography eluting with a suitable mixture of heptane and ethyl acetate or dichloromethane and methanol to afford the titled compound.

Synthetic procedure for:

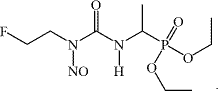

The titled compound is made in an analogous method to that above using commercially available diethyl P-(1-aminoethyl)phosphonate.

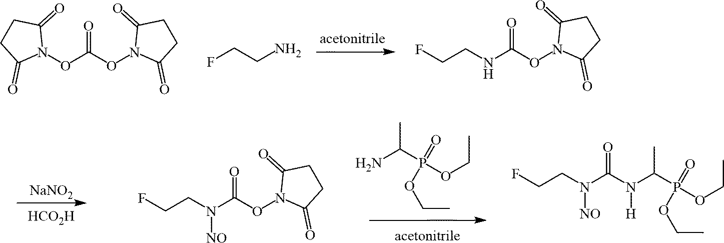

Synthetic procedure for:

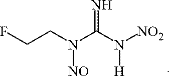

The titled compound is prepared by the following route:

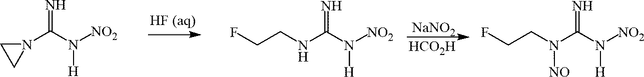

Chem. Pharm. Bull. 1972, 20, 2497-2500 J. Med. Chem. 1980, 23, 848-851

Starting material is prepared according to the procedure described in Chem. Pharm. Bull. 1972, 20, 2497-2500.

Step 1: Acid promoted ring opening of aziridine: A solution of starting material (1 equiv.) in aqueous hydrofluoric acid (0.1 M) is stirred for 1 h. After formation of precipitate is complete solids are collected by vacuum filtration, washed with H2O and used in the next step without further purification.

Step 2: Carbonate N-oxidation: This procedure follows that referenced in J. Med. Chem. 1980, 23, 848-851. Starting nitroguanidinium (1.0 equiv.) is dissolved in formic acid (0.75 M) and cooled to 0° C. in an ice bath. Next, NaNO2 (3.0 equiv.) is added in small portions while maintaining internal temperature below 5° C. The reaction is stirred for 30 minutes below 5° C. Next, the reaction is neutralized to pH 7 with 10% aqueous sodium hydroxide solution. The reaction is extracted with ethyl acetate. Combined organic layers are dried over anhydrous solid sodium sulfate, filtered and concentrated to dryness on a roto-evaporator. The crude residue is purified by silica gel column chromatography eluting with an appropriate mixture of methanol and methylene chloride or chloroform and ethyl acetate to afford the titled compound.

Other compounds within the scope of the disclosure may be prepared by following one of the above-described procedures but using the appropriate starting materials.

### Part B: Additional Compounds

The present disclosure provides a compound of formula (I-1), which is selected from the group consisting of:

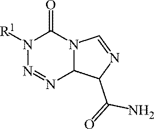

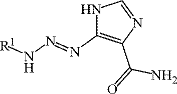

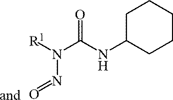

wherein R¹ is selected from the group consisting of optionally substituted C₁-C₆ alkyl and optionally substituted C₁-C₆ haloalkyl, wherein each optional substituent in R¹ is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl. The optional substitution in R¹ can be one or more substituents selected from the group consisting of include F, Cl, Br, I, OR, CN, NO, NO₂, ONO₂, azido, CF₃, OCF₃, R, N(R)₂, SR, SOR, SO₂R, SO₂N(R)₂, SO₃R, and C(O)R, wherein R is independently at each occurrence hydrogen or C₁-C₁₀ alkyl. In various embodiments, R¹ is methyl, ethyl, propyl, or iso-propyl. In various embodiments, the optional substitution in R¹ is at least one substituent selected from the group consisting of F, Cl, Br, CF₃, CF₂H, and CFH2.

In certain embodiments, the compound is selected from the group consisting of:

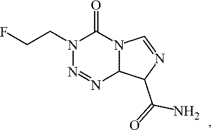

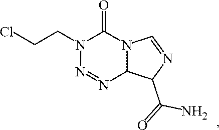

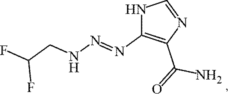

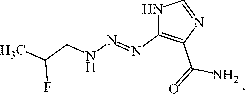

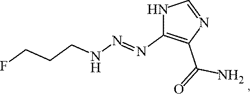

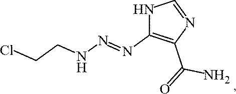

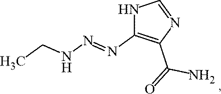

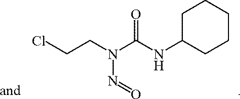

The present disclosure further provides a pharmaceutical composition comprising the compound of the present disclosure and at least one pharmaceutically acceptable carrier.

### Part C: General Considerations for Compounds

The compounds described herein may possess one or more stereocenters, and each stereocenter may exist independently in either the (R)- or (S)-configuration. In certain embodiments, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/ or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the disclosure exist as tautomers. All tautomers are included within the scope of the compounds recited herein.

In certain embodiments, compounds described herein are prepared as prodrugs. A “prodrug” is an agent converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the disclosure are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

### Salts

The compositions described herein may form salts with acids or bases, and such salts are included in the present disclosure. In certain embodiments, the salts are pharmaceutically acceptable salts. The term “salts” embraces addition salts of free acids or free bases that are compositions of the disclosure. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compositions of the disclosure.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compositions of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from the corresponding composition by reacting, for example, the appropriate acid or base with the composition.

### Part D: Pharmaceutical Formulations

Pharmaceutical compositions suitable for use for the compounds and in the methods described herein can include a disclosed compound and a pharmaceutically acceptable carrier or diluent. For example, one aspect of the disclosure provides a subject pharmaceutical composition comprising a therapeutically-effective amount of a compound of formula (I) and a pharmaceutically-acceptable carrier or diluent. Another aspect of the disclosure provides a subject pharmaceutical composition comprising a compound of formula (I) or (I-1) and a pharmaceutically-acceptable carrier or diluent.

The composition may be formulated for intravenous, subcutaneous, intraperitoneal, intramuscular, topical, oral, buckle, nasal, pulmonary or inhalation, ocular, vaginal, or rectal administration. In some embodiments, the compounds are formulated for oral administration. The pharmaceutical composition can be formulated to be an immediate-release composition, sustained-release composition, delayed-release composition, etc., using techniques known in the art.

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, stabilizing agents and the like.

Additionally, the disclosed pharmaceutical compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiment, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Pharmaceutical compositions of the disclosure can be administered in combination with other therapeutics that are part of the current standard of care for cancer.

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or some combination of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington’s Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and combinations thereof. One such preservative is a combination of about 0.5% to 2.0% benzyl alcohol and 0.05% to 0.5% sorbic acid.

The composition may include an antioxidant and a chelating agent which inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Part E: Therapeutic Applications

The present compounds are useful for treatment, amelioration, and/or prevention of any cancer that is MGMT deficient, regardless on its MMR status, but are particularly applicable to treatment of cancers that are both MGMT and MMR deficient.

Cancers (particularly gliomas) often develop MMR deficiency and become resistant and unresponsive to treatment by nitrosoureas. See, for example, D. P. Cahill et al., MSH6 inactivation and emergent temozolamide resistance in human glioblastomas. Clin. Neurosurg. 55, 165-171 (2008). The present compounds, compositions, and methods provide an effective treatment for such cancers.

One aspect of the disclosure provides a method of treating cancer in a subject, the method comprising administering to the subject a compound of the present disclosure or the pharmaceutical composition of the present disclosure.

In certain embodiments, the cancer is a glioma. In certain embodiments, the glioma is resistant to treatment with a DNA methylation agent and/or temozolomide. In certain embodiments, O⁶-methylguanine methyl transferase (MGMT)-silenced tumors are selectively killed.

Genetic instability is a hallmark of cancer and typically arises from mutations in key DNA damage repair and/or reversal proteins (collectively referred to herein as the DNA damage response, (DDR)). Intrinsic DDR defects can be exploited with DDR inhibitors via the concept of synthetic lethality, defined as a loss of viability resulting from the disruption of two genes or pathways, which, if disrupted individually, are non-lethal. Notable examples of synthetic lethal interactions between DDR inhibitors and key tumor-associated DDR defects include: (1) homologous recombination (HR)-defective tumors and inhibitors of poly(ADP)-ribose polymerase (PARP) and polymerase theta (Pol θ); (2) ataxia-telangiectasia mutated (ATM)-mutant tumors and ataxia telangiectasia and Rad3-related (ATR) inhibitors; and (3) mismatch repair (MMR)-deficient tumors and Werner syndrome helicase (WRN) inhibitors. In each of these examples, selective tumor cell killing via the DDR protein inhibitor relies on either the induction or persistence of DNA damage or aberrant DNA structures.

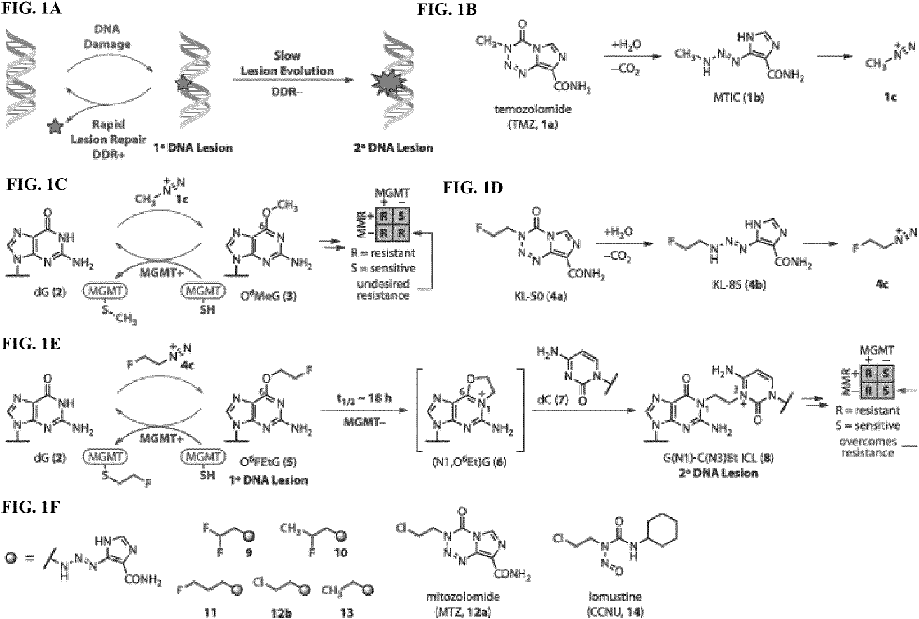

These findings suggested that genotoxins could be tailored used to exploit differential sensitivities arising from specific tumor-associated DDR defects. This approach avoids the need to engage DDR proteins directly, thereby circumventing resistance mechanisms arising from mutations in the ligand binding site, while minimizing off-target effects in healthy, DDR-proficient cells. A mechanistic strategy was conceived wherein a single agent modifies DNA by two successive chemical steps (FIG. 1A). The first chemical reaction is designed to generate a primary DNA lesion that is rapidly removed by healthy, DDR-proficient cells. The second chemical reaction is engineered to slowly transform the primary modification into a more toxic secondary lesion. It was anticipated that if the rate of primary lesion repair were sufficiently rapid in healthy cells, the secondary lesions would accumulate only in the DNA of DDR-deficient tumor cells. This two-step pathway would overcome resistance mechanisms that mitigate the toxicity of a primary lesion, which have been implicated in resistance to various chemotherapies including anthracyclines (impairment of nucleoside excision repair (NER), topoisomerase inhibitors (loss of nonhomologous end joining), as well as antimetabolite and platinum resistance arising from mutations in MMR. Furthermore, delivering these bespoke lesions using established chemotherapy scaffolds could facilitate rapid translation into the clinic, owing to decades of use in cancer patients.

GBM (glioblastoma multiforme) is the most common and devastating form of brain cancer, with a five year survival rate of ~5%. Approximately half of GBMs lack the DDR protein O⁶-methylguanine methyltransferase (MGMT) via promoter hypermethylation. MGMT silencing occurs at an even greater frequency in grade II and III gliomas (over 70% of cases), and these tumors are also largely incurable. Mechanistically, MGMT removes O⁶-alkylguanosine adducts by transferring the alkyl adduct to an active site cysteine via an S_{N}2 displacement. Patients with MGMT-deficient tumors (referred to hereafter as MGMT- tumors) are treated with temozolomide (TMZ, 1a), a prodrug that converts under physiological conditions to the potent methylating agent methyl diazonium (1c), via the intermediacy of 3-methyl-(triazen-1-yl)-imidazole-4-carboxamide (MTIC, 1b) (FIG. 1B). N7-Methylguanosine and N3-methyladenosine are the major sites of methylation (70% and 9% respectively) but are readily resolved by the base excision repair (BER) pathway.

In contrast, though O⁶-methylguanosine (O⁶MeG, 3) adducts derived from TMZ (1a) only comprise ~5% of addition products, these lesions persist in the genome of MGMT- tumors (but are readily reversed by healthy (MGMT+) cells) (FIG. 1C). O⁶MeG (3) residues are thought to induce formation of DNA double-strand breaks (DSBs) and tumor cell death by an MMR-dependent mechanism. MGMT status is a predictive biomarker for initial response to TMZ (1a) in GBM, with a significant overall survival (OS) benefit in the up-front setting for patients with these cancers. However, it is now well-established that acquired clinical resistance to TMZ (1a) by MMR mutations abrogates its toxicity, leading to recurrent GBM and death in nearly all patients. TMZ (1a) is also frequently utilized as adjuvant therapy for grade III and high-risk grade II gliomas; however, it remains non-curative, with recurrences typically occurring over 2-10 years. In approximately 80% of patients, recurrences coincide with transformation to higher grade tumors resistant to TMZ (1a) and harboring a distinct hypermutation signature secondary to MMR deficiency, resulting in reduced survival.

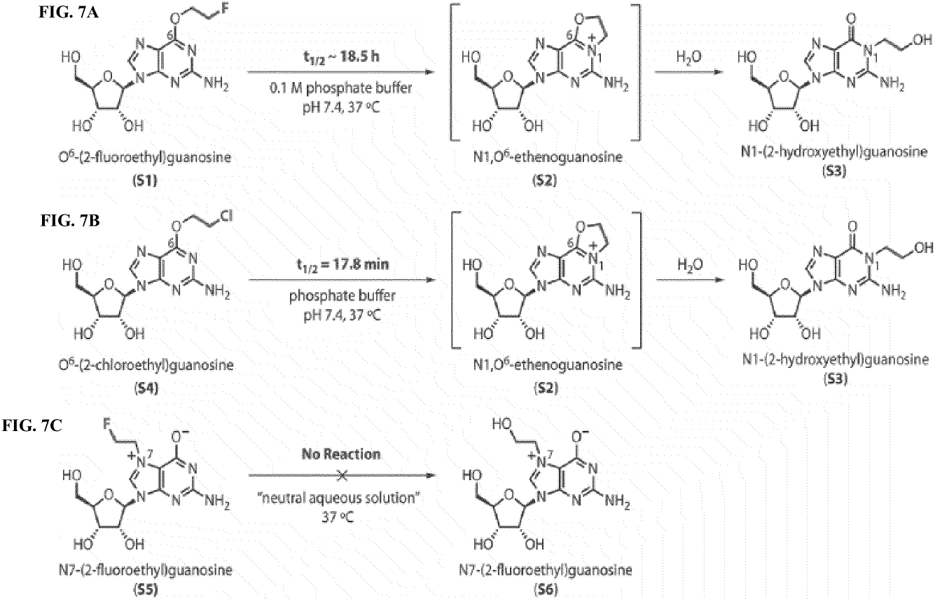

In various embodiments, the strategy outlined in FIG. 1A can be used to develop agents that overcome the resistance associated with MMR loss while maintaining TMZ’s (1a) selectivity for MGMT-silenced tumors. These agents would deposit a primary lesion susceptible to S_{N}2-mediated removal by MGMT that could undergo a further chemical transformation to a secondary lesion capable of killing MGMT-deficient tumor cells in an MMR-independent manner. To maintain the therapeutic index between MGMT- tumor cells and MGMT+ healthy cells, the primary legion must undergo MGMT-mediated repair faster than it undergoes transformation to the secondary lesion. With these considerations in mind, it was hypothesized that an agent capable of depositing a 2-fluoroethyl lesion at O⁶-guanine would prove ideal as O⁶-(2-fluoroethyl)guanosine (S1) is known to hydrolyze slowly to N1-(2-hydroxyethyl)guanosine (S3) with a half-life of 18.5 h (37° C., pH 7.4) (FIG. 7A). Mechanistically, this occurs via N1 displacement of the pendent fluoride to provide the N1,O⁶-ethanoguanosine intermediate S2 which undergoes ring-opening nucleophilic attack by water to give S3. It was reasoned that the G(N1)-C(N3) inter-strand cross-link (ICL) 8 may form by conversion of O⁶FEtG (5) to the

N1,O⁶-ethanoguanine intermediate 6 followed by ring-opening by N3 of the complementary cytosine base (7; FIG. 1E). As MGMT reacts rapidly with alkylated DNA (a second-order rate constant of 1×10⁹ M⁻¹•min⁻¹ was measured using methylated calf thymus DNA as substrate) and can act upon a wide range of O⁶-alkylguanine substrates. It was anticipated that MGMT-proficient cells should repair the O⁶FEtG lesion (5) before it transforms into ICL 8.

### Methods of Treatment, Amelioration, and/or Prevention

In the present method, a compound of formula (I) is administered to a patient (e.g., a human patient) suffering from an MGMT deficient cancer. The method can treat, ameliorate, and/or prevent an occurrence or recurrence of any cellular proliferative disorder described herein, as well as can treat, ameliorate, and/or prevent one or more symptoms of any cellular proliferative disorder described herein. In another embodiment, the present method comprises administration of a therapeutically-effective amount of the compound of formula (I) to a patient suffering from an MGMT deficient, MMR deficient cancer, particularly a glioma. In some embodiments, the therapeutically effective amount of the compound is administered together with a pharmaceutically acceptable carrier. Suitable pharmaceutically acceptable carriers are well-known in the art, as discussed infra. A typical route of administration is oral, but other routes of administration are possible, as is well understood by those skilled in the medical arts. Administration may be by single or multiple doses. The amount of compound administered and the frequency of dosing may be optimized by the physician for the particular patient.

In addition to gliomas such as glioblastoma multiforme and brain lower grade glioma, the present method and compounds are useful to treat urothelial cancer, breast invasive carcinoma, colon adenocarcinoma, head and neck tumor (SCC), lung adenocarcinoma, rectum adenocarcinoma, acute myeloid leukemia, Hodgkin’s lymphoma, non-Hodgkin’s lymphoma, melanoma, and lung cancer and particularly the above cancers that are MGMT deficient regardless of MMR status or are MGMT and MMR deficient.

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pats. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer’s solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically-or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pats. Nos. 6,340,475, 6,488,962, 6,451,808, 5,972,389, 5,582,837, and 5,007,790. Additional dosage forms of this disclosure also include dosage forms as described in U.S. Pat. Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Additional Aspects of Administration/Dosage/Formulations

Routes of administration of any of the compounds and/or compositions of the disclosure include oral, nasal, rectal, intravaginal, parenteral (e.g., IM, IV and SC), buccal, sublingual or topical. The regimen of administration may affect what constitutes an effective amount. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a subject, preferably a mammal, more preferably a human, may be carried out using known procedures, at dosages and for periods of time effective to treat the disorder involving overexpression of methylene tetrahydrofolate dehydrogenase 2 (MTHFD2) in a subject. An effective amount of the therapeutic compound necessary to achieve a therapeutic effect may vary according to factors such as the state of the disease or disorder in the subject; the age, sex, and weight of the subject; and the ability of the therapeutic compound to treat the disease or disorder in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound useful within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this disclosure may be varied so as to obtain an amount of the active ingredient that is effective to achieve the desired therapeutic response for a particular subject, composition, and mode of administration, without being toxic to the subject.

In particular, the selected dosage level depends upon a variety of factors, including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian may start doses of the compounds useful within the disclosure employed in the pharmaceutical composition at levels lower than that required in order to achieve the desired therapeutic effect and gradually increase the dosage until the desired effect is achieved.

In certain embodiments, the carboxypeptidase is glucarpidase (CPG2) which is commercially available as VORAXAZE® from Protherics Medicine Limited/BTG Specialty Pharmaceuticals. The specified dose for administration to deplete patient methotrexate (MTX) in a rescue situation is 50 units/kg. In certain embodiments, the dose used to deplete folate in the subject is 50 units/kg. In other embodiments, the dose used to deplete folate in the subject is greater than 50 units/kg. In yet other embodiments, lower doses from 1 to 50 units/kg given over the course of days or weeks may be used to achieve folate depletion in the subject. A non-limiting example including CPG2 administration by injection/intravenous drop several times a week, for example 2, 3, or 4 times a week for 1 to 8 weeks.

In certain embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the disclosure are dictated by and directly dependent on the unique characteristics of the therapeutic compound and the particular therapeutic effect to be achieved, and the limitations inherent in the art of compounding/formulating such a therapeutic compound for the treatment of a disorder involving overexpression of methylene tetrahydrofolate dehydrogenase 2 (MTHFD2) in a subject.

In certain embodiments, the compositions of the disclosure are formulated using one or more pharmaceutically acceptable excipients or carriers. In certain embodiments, the pharmaceutical compositions of the disclosure comprise a therapeutically effective amount of a compound useful within the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the subject in dosages that range from one to five times per day or more. In other embodiments, the compositions of the disclosure are administered to the subject in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any subject are determined by the attending physical taking all other factors about the subject into account.

Compounds useful within the disclosure for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In certain embodiments, the dose of a compound useful within the disclosure is from about 1 mg and about 2,500 mg. In other embodiments, a dose of a compound useful within the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in certain embodiments, a dose of a second compound, as described herein, is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments therebetween.

In certain embodiments, the present disclosure is directed to a packaged pharmaceutical composition comprising a container holding a therapeutically effective amount of a compound useful within the disclosure, alone or in combination with a second pharmaceutical agent; and instructions for using the compound to treat, prevent, and/or ameliorate a disorder involving overexpression of methylene tetrahydrofolate dehydrogenase 2 (MTHFD2) in a subject.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multilayer tablet comprising a layer providing for the delayed release of one or more compounds useful within the disclosure, and a further layer providing for the immediate release of a medication for a disorder involving overexpression of methylene tetrahydrofolate dehydrogenase 2 (MTHFD2) in a subject. Using a wax/pH-sensitive polymer mix, a gastric insoluble composition may be obtained in which the active ingredient is entrapped, ensuring its delayed release.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds of the present disclosure may be administered in the form of microparticles, for example, by injection or in the form of wafers or discs by implantation.

In a preferred embodiment of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure will depend on the age, sex and weight of the subject, the current medical condition of the subject and the nature of the disorder involving overexpression of methylene tetrahydrofolate dehydrogenase 2 (MTHFD2) being treated. The skilled artisan will be able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

The compounds for use in the method of the disclosure may be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

### Therapeutically Effective Doses and Dosing Regimens

In some embodiments, the therapeutically effective dose of the compound may be administered every day, for 21 days followed by a 7 day rest, every 7 days with a 7 day rest in between each dosage period, or for 5 continuous days followed by a 21 day rest, in each instance referring to a 28 day dosage cycle.

The therapeutically effective dose of compound administered to the patient (whether administered in a single does or multiple doses) should be sufficient to treat the cancer. Such therapeutically effective amount may be determined by evaluating the symptomatic changes in the patient.

Exemplary doses can vary according to the size and health of the individual being treated, the condition being treated, and the dosage regimen adopted. In some embodiments, the effective amount of a disclosed compound per 28 day dosage cycle is about 1.5 g/m²; however, in some situations the dose may be higher or lower - for example 2.0 g/m² or 1.0 g/m². The daily dose may vary depending on (inter alia) the dosage regimen adopted. For example, if the regimen is dosing for five days followed by a 21 day rest and the total dosage per 28 day cycle is 1.0 g/m², then the daily dose would be 200 mg/m². Alternatively, if the regimen is dosing for 21 days followed by a 5 day rest and the total dosage per 28 day cycle is 1.6 g/m², then the daily dose would be 75 mg/m². Similar results would obtain for other dosage regimens and total 28 day doses.

Compounds of the disclosure for administration may be in the range of from about 1 µg to about 7,500 mg, about 20 µg to about 7,000 mg, about 40 µg to about 6,500 mg, about 80 µg to about 6,000 mg, about 100 µg to about 5,500 mg, about 200 µg to about 5,000 mg, about 400 µg to about 4,000 mg, about 800 µg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 µg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound’s ability to perform its intended function, e.g., treating, preventing, or reducing a disease or disorder in a patient.

The disclosed methods of treatment may also be combined with other known methods of treatment as the situation may require.

Therapeutically effective doses and dosing regimens of the foregoing methods may vary, as would be readily understood by those of skill in the art. Dosage regimens may be adjusted to provide the optimum desired response. For example, in some embodiments, a single bolus dose of the compound may be administered, while in some embodiments, several divided doses may be administered over time, or the dose may be proportionally reduced or increased in subsequent dosing as indicated by the situation.

### Controlled Release Formulations and Drug Delivery Systems:

In certain embodiments, the compositions and/or formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Without wishing to be bound by theory, the disclosed compounds, such as compounds of formula (I), act as bifunctional alkylation agents in a two-step process. The first reaction generates a primary DNA lesion (alkylation) that is rapidly removed by healthy MGMT-proficient cells. The second reaction slowly transforms the primary modification (alkylation) into a more toxic lesion via a unimolecular process. Thus, Applicant believes the disclosed compounds first alkylate O6-guanine and thereafter evolve slowly to more toxic inter-strand cross link (ICL), thereby establishing an MMR-independent method to amplify the therapeutic impact of MGMT deficiency.

The present compounds may be synthesized according to the synthetic schemes and reference methods described above.

The pharmaceutical activity of the subject compounds may be evaluated in the following assays:

Short-term cell viability assay: On day 1, Ln229 isogenic cells of varying MGMT and MMR status are seeded in 96 well format at a density of 2000 cells/well in 100 µL of DMEM media and allowed to adhere overnight. On day 2, a drug master plate is made with 100× the desired maximal concentration of test compound and serially diluted by 2 until 100× the minimal desired concentration, with one DMSO control. Then daughter plates are created with varied concentrations from 3× the minimal concentration to 3× the maximal desired concentration. Afterwards, 50 µL of daughter drug plate is added to 100 µL of the seeded cells for a final concentration of 1× in triplicate. Cells are allowed to grow for 6 days. On day 7, the cells are fixed, and stained with Hoechst nuclear dye and imaged to determine growth inhibition.

Clonogenic Survival Assay: Isogenic glioma (Ln 229) cells are pretreated with the test drug in culture for 48-72 hours at the specified dilutions. Cells are then transferred in media without drug to 6-well plates in triplicate at 3-fold dilutions ranging from 9,000 to 37 cells per well. After 14 days, plates are washed with PBS and stained with crystal violet. Colonies are counted by hand. Counts are normalized to plating efficiency of the corresponding treatment condition.

Xenograft Experiments: All animal studies are approved by the Institutional Animal Use and Care Committee and performed in accordance with the Guide for the Care and Use of Laboratory Animals. LN229 WT and LN229-MSH2⁻ cell lines are maintained in DMEM media supplemented with 10% fetal bovine serum. Three-four-week-old female athymic nude Foxnnu mice are obtained from Envigo and each mouse is inoculated subcutaneously with tumor cells (4.5-5 × 10⁶) in 0.1 ml of PBS with Matrigel (1:1). Wild type cells are injected on the right flank and mutant cells are injected on the left flank. The tumors are then grown to a mean size of approximately 50-100 mm3 and the mice are then split into groups and treated as detailed in FIG. 7. Gavage doses of 5 mg/kg of test compound are prepared by diluting stocks in DMSO with 10% cyclodextrin. Compound is administered each day of dosing at a volume of 100 µL/mouse. Mice are treated for 3 weeks with dosing on Mondays, Wednesdays and Fridays. Tumors are measured 3 times a week during treatment and during the washout period of 2 weeks. Xenograft tumors are measured by calipers and volume is calculated using the equation for ellipsoid volume: Volume = 0.523 × (length) × (width)². Statistical Analysis: Analysis of variance (ANOVA) is used to test for significant differences between groups. Post-hoc Bonferroni multiple comparison test analysis is used to determine significant differences among means. All statistical analysis is accomplished using Graph Pad Prism 8.2.0 software.

The clonogenic survival assay is a well-recognized assay with high prediction of utility of cancer treatment compounds. See, for example, Fiebig et al. - Clonogenic assay with established human tumor xenografts: correlation of in vitro to in vivo activity as a basis for anticancer drug discovery - European J. Cancer, 40 (2004) 802-820. Although the short-term cell viability assay is recognized to be not as predictive of clinical usefulness as the clonogenic survival assay, negative results in the short-term cell viability assay are understood to indicate the tested compound is not a candidate for further study.

All references cited herein are incorporated herein by reference as if included in their entirety.

In the description and claims of this specification the word “comprise” and variations of that word, such as “comprises” and “comprising” are not intended to exclude other features, additives, components, integers or steps but rather, unless otherwise stated explicitly, the scope of these words should be construed broadly such that they have an inclusive meaning rather than an exclusive one.

Although the compounds, compositions, and methods of the disclosure have been described in the present disclosure by way of illustrative examples, it is to be understood that the disclosure is not limited thereto and that variations can be made as known by those skilled in the art without departing from the teachings of the disclosure defined by the appended claims.

### Part F: Enumerated Embodiments

The following exemplary embodiments are provided, the numbering of which is not to be construed as designating levels of importance:

Embodiment 1 provides a compound of formula (I-1), which is selected from the group consisting of:

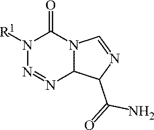

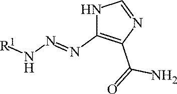

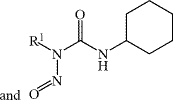

wherein R¹ is selected from the group consisting of optionally substituted C₁-C₆ alkyl and optionally substituted C₁-C₆ haloalkyl, wherein each optional substituent in R¹ is independently selected from the group consisting of halogen, C₁-C₃ haloalkyl, C₁-C₃ alkoxy, C₁-C₃ haloalkoxy,
C₁-C₃ alkyl, C₂-C₆ alkenyl, benzyl, phenyl, and naphthyl, and C₂-C₁₂ heterocyclyl;
or a salt, solvate, tautomer, or isotopologue thereof.

Embodiment 2 provides the compound of Embodiment 1, which is selected from the group consisting of

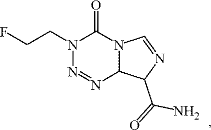

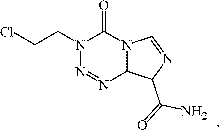

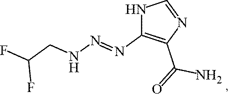

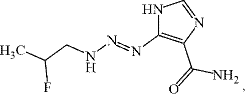

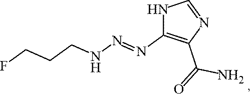

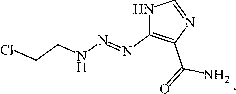

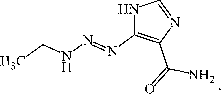

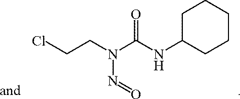

Embodiment 3 provides a pharmaceutical composition comprising the compound of Embodiment 1 or 2 and at least one pharmaceutically acceptable carrier.

Embodiment 4 provides a method of treating cancer in a subject, the method comprising administering to the subject a compound of Embodiment 1 or 2 or the pharmaceutical composition of Embodiment 3.

Embodiment 5 provides the method of Embodiment 4, wherein the cancer is a glioma.

Embodiment 6 provides the method of Embodiment 4 or 5, wherein the glioma is resistant to treatment with a DNA methylation agent and/or temozolomide.

Embodiment 7 provides the method of any one of Embodiments 4-6, wherein O⁶-methylguanine methyl transferase (MGMT)-silenced tumors are selectively killed.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Materials and Methods

General Chemical Experimental Procedures. All reactions were generally performed in single-neck, flame dried round-bottom flasks fitted with rubber septa under a positive pressure of argon, unless otherwise specified. Air- and moisture-sensitive liquids were transferred via syringe or stainless-steel cannula. Organic solutions were concentrated by rotary evaporation at 31° C., unless otherwise noted. Flash-column chromatography was performed employing silica gel (SiliaFlash® P60, 60 Å, 40-63 µm particle size) purchased from Silicycle (Quebec, Canada). Analytical thin-layered chromatography (TLC) was performed using glass plates pre-coated with silica gel (250 µm, 60 Å pore size) embedded with a fluorescent indicator (254 nm). TLC plates were visualized by exposure to ultraviolet (UV) light.

Chemical Materials. Commercial solvents, chemicals, and reagents were used as received with the follow exceptions. Dichloromethane, tetrahydrofuran, and toluene were purified according to the method of A. B. Pangborn, M. A. Giardello, R. H. Grubbs, R. K. Rosen, F. J. Timmers, Safe and Convenient Procedure for Solvent Purification. Organometallics 15, 1518-1520 (1996). Triethylamine was distilled from calcium hydride under an atmosphere of nitrogen immediately prior to use. N,N-Di-iso-propylethylamine was distilled from calcium hydride under argon immediately prior to use. The diazonium S7, the imidazolyl triazene 1b, the imidazolyl triazene 4b, the imidazolyl triazene 9, the imidazolyl triazene 12b, and the imidazolyl triazene 13 were synthesized according to published procedures.

Chemistry Instrumentation. Unless indicated otherwise, proton nuclear magnetic resonance (¹H NMR) were recorded at 400 or 600 megahertz (MHz) at 23° C., unless otherwise noted. Chemical shifts are expressed in parts per million (ppm, δ scale) downfield from tetramethylsilane and are referenced to residual proton in the NMR solvent ((CD₃)SO(CHD₂), δ 2.50). Data are represented as follows: chemical shift, multiplicity (s = singlet, d = doublet, t = triplet, q = quartet, m = multiplet and/or multiple resonances, b = broad, app = apparent), coupling constant in Hertz (Hz), integration, and assignment. Proton-decoupled carbon nuclear magnetic resonance spectra (¹³C NMR) were recorded at 150 MHz at 23° C., unless otherwise noted. Chemical shifts are expressed in parts per million (ppm, (δ scale) downfield from tetramethylsilane and are referenced to the carbon resonances of the solvent (DMSO-d₆, δ 39.52). ¹H-¹H gradient-selected correlation spectroscopy (COSY), ¹H-¹³C heteronuclear single quantum coherence (HSQC), and ¹H-¹³C gradient-selected heteronuclear multiple bond correlation (gHMBC) were recorded at 600 MHz at 23° C., unless otherwise noted. Carbon-decoupled fluorine nuclear magnetic resonance spectra (¹⁹F NMR) were recorded at 396 MHz at 23° C., unless otherwise noted. Chemical shifts are expressed in parts per million (ppm, δ scale) downfield from tetramethylsilane. Attenuated total reflectance Fourier transform infrared (ATR-FTIR) spectra were obtained using a Thermo Electron Corporation Nicolet 6700 FTIR spectrometer referenced to a polystyrene standard. Data are represented as follows: frequency of absorption (cm⁻¹), intensity of absorption (s = strong, m = medium, w = weak, br = broad). Analytical liquid chromatography-mass spectroscopy (LCMS) was performed on a Waters instrument equipped with a reverse-phase C₁₈ column (1.7 µm particle size, 2.1 × 50 mm). Samples were eluted with a linear gradient of 5% acetonitrile-water containing 0.1% formic acid→100% acetonitrile containing 0.1% formic acid over 0.75 min, followed by 100% acetonitrile containing 0.1% formic acid for 0.75 min, at a flow rate of 800 µL/min. HRMS were obtained on a Waters UPLC/HRMS instrument equipped with a dual API/ESI high resolution mass spectrometry detector and photodiode array detector.

Biological Materials. Temozolomide (TMZ, 1a), lomustine (14), O⁶-benzylguanine (O⁶BG), doxorubicin, and olaparib were purchased from Selleck Chemicals. Methylmethane sulfonate (MMS) was purchased from Alfa-Aesir. Mitozolomide (MTZ, 12a) was purchased from Enamine. Mitomycin C (MMC), N-ethylmaleimide (NEM), N-acetyl-L-cysteine (NAC), and cisplatin were purchased from Sigma. TMZ (1a, 100 mM stock), O⁶BG (100 mM stock), MTZ (12a, 100 mM stock), MMS (500 mM stock) and NAC (100 mM stock) were dissolved in DMSO and stored at -80° C. MMC (10 mM stock), lomustine (14, 100 mM stock), doxorubicin (10 mM stock), and olaparib (18.3 mM stock) were dissolved in DMSO and stored at -20° C. NEM (400 mM stock) was dissolved in EtOH and stored at -20° C. Cisplatin (5 mM stock) was dissolved in H₂O and stored at 4° C. for up to 7 days.

Cell Culture. LN229 MGMT- and MGMT+ cell lines were a gift from B. Kaina (Johannes Gutenberg University Mainz, Mainz, Germany) and grown in DMEM with 10% FBS (Gibco). DLD1 BRCA2+/- and BRCA2-/- cell lines (Horizon Discovery, Cambridge, UK) were grown in RPMI 1640 with 10% FBS. HCT116 MLH1-/- and HCT116+Chr3 cell lines were a gift from T. Kunkel (National Institute of Environmental Health Sciences, Durham, NC) and grown in DMEM with 10% FBS, with 0.5 µg/mL G418 (Sigma) for HCT116+Chr3 cells. PD20 cell lines complemented with empty vector (+EV), wildtype FANCD2 (+FD2), or K561R ubiquitination-mutant FANCD2 (+KR) were a gift from G. Kupfer and P. Glazer (Yale University, New Haven, CT) and growth in DMEM with 10% FBS. PEO1 and PEO4 cell lines were a gift from T. Taniguchi (Fred Hutchinson Cancer Research Center, Seattle, WA) and were grown in DMEM with 10% FBS. BJ fibroblasts (normal human fibroblast cells) were purchased from ATCC (CRL-2522) and grown in DMEM with 10% FBS. NER isogenic MEFs were a gift from F. Rogers (Yale University, New Haven, CT) and were grown in DMEM with 10% FBS. All human cell lines were validated by short tandem repeat profiling (excluding BJ fibroblasts which were used within 6 passages of receiving from ATCC) and confirmed negative for mycoplasma by quantitative RT-PCR.

MMR Protein shRNA Knockdown. pGIPZ lentiviral shRNA vectors targeting MSH2, MSH6, MLH1, PMS2, and MSH3 were purchased from Horizon Discovery (Table S2). Lentiviral particles were produced in HEK293T cells via co-transfection with lentiviral shRNA plasmid, pCMV-VSV-G envelope plasmid (Addgene, #8454) and psPAX2 packaging plasmid (Addgene, #12260), using Lipofectamine 3000 Reagent (Invitrogen, L3000001) per manufacturer’s protocol. Viral particles were harvested 48 h post-transfection and used to transduce LN229 MGMT+/- cells in the presence of 8 µg/mL polybrene. Selection of pooled cells with lentiviral expression was established with 1 µg/mL puromycin 48 h post-transduction for 3 to 4 days. Single cell cloning was performed by limiting dilution and protein knockdown was confirmed by western blotting.

TABLE 1

pGIPZ Lentivral shRNA Vectors for MMR protein knockdown

| | | |
|---|---|---|
| Protein Target | pGIPZ Human Lentiviral shRNA Clone | Mature Antisense Sequence |
| MSH2 | RHS4430-200305416 | TTACTAAGCACAACACTCT (SEQ ID NO: 1) |
| MSH6 | RHS4430-200281418 | TACACATTACTTTGAATCC (SEQ ID NO: 2) |
| MLH1 | RHS4430-200268977 | AACTGAGAAACTAATGCCT (SEQ ID NO: 3) |
| PMS2 | RHS4430-200253216 | TTCACAGCTACATCAACCT (SEQ ID NO: 4) |
| MSH3 | RHS4430-200158125 | TTTCTTGCAAATGCATTCG (SEQ ID NO: 5) |

Western Blotting. For phospho-protein analysis experiments, cells were lysed in 1X RIPA buffer (Cell Signaling Technology, #9806) supplemented with 1X Protease Inhibitor Cocktail (Roche) and 1X PhosSTOP Phosphatase Inhibitor Cocktail (Sigma). For all other western blot analyses, cells were lysed in lysis buffer (50 mM HEPES, 250 mM NaCl, 5 mM EDTA, 1% NP-40) supplemented with 1X Protease Inhibitor Cocktail (Roche). The de-ubiquitination inhibitor N-ethylmaleimide (NEM, 4 mM) was added in FANCD2 ubiquitination analysis experiments. Proteins were separated using NuPAGE 4-12% Bis-Tris or 3-8% Tris-Acetate Gels (Invitrogen) and transferred to Immobilon-P PVDF membrane (Millipore) for western blotting. Membranes were blocked with 5% milk in TBS-T for 1 h prior to primary antibody addition overnight at 4° C. Primary antibodies were used under the following conditions: mouse anti-CHK1 (Cell Signaling Technology, #2360), 1/1000 in 5% milk; rabbit anti-CHK2 (Cell Signaling Technology, #6334), 1/1000 in 5% BSA; rabbit anti-FANCD2 (Cell Signaling Technology, #16323), 1/1000 in 5% BSA; HRP-conjugated anti-GAPDH (ProteinTech HRP-60004), 1/10,000 in 5% milk; rabbit anti-MGMT (Cell Signaling Technology, #2739), 1/1000 in 5% BSA; rabbit anti-MLH1 (Cell Signaling Technology, #4256), 1/1000 in 5% BSA; mouse anti-MSH2 (Cell Signaling Technology, #2850), 1/1000 in 5% milk; mouse anti-MSH3 (BD Biosciences, BD611390), 1/500 in 5% milk; mouse anti-MSH6 (BD Biosciences, BD610918), 1/1000 in 5% milk; rabbit anti-phospho-CHK1 (S345) (Cell Signaling Technology, #2341), 1/1000 in 5% BSA; rabbit anti-phospho-CHK2 (T68) (Cell Signaling Technology, #2661), 1/1000 in % BSA; mouse anti-PMS2 (Santa Cruz, sc-25315), 1/100 in 5% milk; mouse anti-Vinculin (Santa Cruz, sc-25336), 1/1000 in 5% milk. Anti-mouse IgG HRP-conjugated antibody (Cell Signaling Technology, #7076) and anti-rabbit IgG HRP-conjugated antibody (Cell Signaling Technology, #93702) were added at 1/5000 in 5% milk for 1 h. Chemiluminescence detection was performed with Clarity Max Western ECL Substrate (Bio-Rad) and blots were imaged on a ChemiDoc XRS+ Molecular Imager (Bio-Rad). Where shown, bands were quantified using ImageJ software.

Short-term Cell Viability Assay. Cells were seeded in 96-well plates at 1000 or 2000 cells/well and allowed to adhere at 23° C. for 60 min and then incubated overnight at 37° C. Cells were treated with indicated concentrations of compounds in triplicate for 4-6 days prior to fixation with 3.7% paraformaldehyde and nuclear staining with 1 µg/mL Hoechst 33342 dye. Cells were imaged on a Cytation 3 imaging reader (BioTek) and quantified using CellProfiler software.

Clonogenic Cell Survival Assay. Cells were trypsinized, washed, counted, and diluted in a medium containing various concentrations of drug. They were then immediately seeded in six-well plates in triplicate at three-fold dilutions, ranging from 9000 to 37 cells per well. Depending on colony size, these plates were kept in the incubator for 10 to 14 days. After incubation, colonies were washed in phosphate-buffered saline (PBS), stained with crystal violet, counted, and quantified.

IR Alkaline Comet Assay. Assay was performed utilizing the CometAssay Kit (Trevigen) according to the alkaline assay protocol, with the addition of slide irradiation post-lysis. Cells were trypsinized, washed with 1X PBS, added to melted Comet LMAgarose (Trevigen), and spread on Trevigen CometSlides at a density of 1000 cells per sample in 50 µL. Lysis solution (Trevigen) with 10% DMSO was added overnight at 4° C. Slides were removed from lysis buffer and irradiated to 0 or 10 Gy using an XRAD 320 X-Ray System (Precision X-Ray) at 320 kV, 12.5 mA, and 50.0 cm SSD, with a 2 mm Al filter and 20 cm × 20 cm collimator. Slides were then placed in alkaline buffer (200 mM NaOH, 1 mM EDTA) for 45 min, followed by electrophoresis in 850 mL alkaline buffer for 45 min at 4° C. Slides were washed and stained with SYBR gold (Invitrogen) per Trevigen assay protocol. Slides were imaged on a Cytation 3 imaging reader (BioTek), and comets were analyzed using CometScore 2.0 software (TriTek).

Genomic DNA Denaturing Gel Electrophoresis. Assay was adapted from N. Bossuet-Greif et al., The Colibactin Genotoxin Generates DNA Interstrand Cross-Links in Infected Cells. mBio 9, (2018). Cells were trypsinized, washed with 1X PBS, and stored at -80° C. prior to processing. Genomic DNA was extracted with the DNeasy Blood & Tissue Kit (Qiagen) per kit protocol. A 0.7% agarose gel was prepared in 100 mM NaCl-2mM EDTA (pH 8) and soaked in 40 mM NaOH-1 mM EDTA running buffer for 2 h. Genomic DNA (400 ng/well) was then loaded in 1X BlueJuice loading buffer (Invitrogen) and subjected to electrophoresis at 2 V/cm for 30 min, followed by 3 V/cm for 2 h. The gel was neutralized in 150 mM NaCl-100 mM Tris (pH 7.4) for 30 min, twice, and then stained with 1X SYBR Gold in 150 mM NaCl-100 mM Tris (pH 7.4) for 90 min. Imaging was performed on a ChemiDoc XRS+ Molecular Imager (Bio-Rad).

Plasmid Linearization Assay. To set up the linearization reactions, 20 units of EcoRI-HF (New England Biolabs) was mixed with 20 µg 2686 bp pUC19 vector DNA in CutSmart buffer (New England Biolabs), pH 7.9, in a total volume of 1000 µL for 30 min at 37° C. The CutSmart buffer contains 50 mM potassium acetate, 20 mM Tris acetate, 10 mM magnesium acetate, and 100 µg/mL BSA. The reacted DNA was then purified using PCR cleanup kit and quantified using the NanoDrop One (Thermo Fisher). The DNA was then stored at -20° C. before use in in vitro DNA cross-linking assays or melting temperature analysis.

In Vitro DNA Cross-linking Assays. Linearized pUC19 DNA, prepared as described above, was used for in vitro DNA cross-linking assays. For each condition, 200 ng of linearized pUC19 DNA (15.4 µM base pairs) was incubated with the indicated concentration of drug in 20 µL. Drug stock concentrations were made in DMSO such that each reaction contained a fixed 5% DMSO concentration. Reactions were conducted in 100 mM Tris buffer (pH 7.4). Cisplatin (Sigma) and DMSO vehicle were used as positive and negative controls, respectively. Reactions were conducted between 3-96 h at 37° C. The DNA was stored at -80° C. until electrophoretic analysis. For gel electrophoresis, DNA concentration was preadjusted to 10 ng/µL. Five microliters (50 ng) of the DNA solution was removed and mixed with 1.5 µL of 6× purple gel loading dye, no SDS, and loaded onto 1% agarose Tris Borate EDTA TBE gels. For denaturing gels, 5 µL (50 ng) of the DNA solution was removed and mixed with 15 µL of 0.2% denaturing buffer (0.27% sodium hydroxide, 10% glycerol, and 0.013% bromophenol blue) or 0.4% denaturing buffer (0.53% sodium hydroxide, 10% glycerol, and 0.013% bromophenol blue) in an ice bath. The mixed DNA samples were denatured at 4° C. for 5 min and then immediately loaded onto a 1% agarose Tris Borate EDTA (TBE) gel. All gel electrophoresis was conducted at 90 V for 2 h (unless otherwise noted). The gel was stained with SYBR Gold (Invitrogen) for 2 h.

EndoIV Depurination Assay. For each condition, 200 ng of supercoiled pUC19 DNA (15.4 µM base pairs) was incubated with the indicated concentration of drug in 20 µL for 3 hours. Drug stock concentrations were made in DMSO such that each reaction contained a fixed 5% DMSO concentration. Reactions were conducted in 100 mM Tris buffer (pH 7.4). For each EndoIV reaction, 50 ng of processed DNA was mixed with 20 units of EndoIV in NEBuffer 3.1 (New England Biolabs), pH 7.9, in a total volume of 20 µL for 16-20 h (unless otherwise noted) at 37° C. The NEBuffer 3.1 contained 100 mM sodium chloride, 50 mM Tris-HCl, 10 mM magnesium chloride, and 100 µg/mL BSA. For each negative control, 50 ng of processed DNA was mixed with NEBuffer 3.1, pH 7.9, in a total volume of 20 µL for 16-20 h (unless otherwise noted) at 37° C. Following completion of the experiment, the DNA was stored at -20° C. before electrophoretic analysis.

Melting Temperature Assay. Linearized pUC19 DNA (750 ng), prepared as described above, was incubated with the indicated concentration of either MMS or KL-50 (4a) adjusted in a final volume of 18 µL in 100 mM Tris buffer (pH 7.4) for 3 h. Drug stock concentrations were made in DMSO such that each reaction contained a fixed 5% DMSO concentration. Afterwards, 1 µL each of 20x SYBR Green dye (Invitrogen) and 20x ROX reference dye (Invitrogen) was added and melting temperature analysis was run on a StepOnePlus RT PCR System (Applied Biosciences) to generate melting temperature curves.

Immunofluorescence Foci Assays. High-throughput immunofluorescence foci assays were performed at the Yale Center for Molecular Discovery (YCMD). Cells were seeded at 2000 cells/well in black polystyrene flat bottom 384-well plates (Greiner Bio-One) and allowed to adhere overnight. Compound addition was performed utilizing a Labcyte Echo 550 liquid handler (Beckman Coulter), with 6 replicates per test condition and 12 replicates per control condition. Following drug incubation, cells were fixed and stained for phospho-SER139-H2AX (yH2AX), 53BP1, or phospho-SER33-RPA2 (pRPA) as follows.

γH2AX protocol: Cells were fixed with 4% paraformaldehyde in 1X PBS for 15 min, washed twice with 1X PBS, incubated in extraction buffer (0.5% Triton X-100 in 1X PBS) for 10 min, washed twice with 1X PBS, and incubated in blocking buffer (Blocker Casein in PBS, Thermo Scientific + 5% goat serum, Life Technologies) for 1 h. Mouse anti-phospho-histone H2A.X (Ser139) antibody (clone JBW301, Millipore, 05-636) was added 1/1000 in blocking buffer at 4° C. overnight. After washing with 1X PBS, cells were incubated with goat anti-mouse IgG (H+L) highly cross-adsorbed secondary antibody, Alexa Fluor 647 (Invitrogen, A-21236) 1/500 and with 1 µg/mL Hoechst nucleic acid dye in blocking buffer for 2 h, and then washed with 1X PBS.

53BP1 protocol: Assay was performed as previously described in Y. V. Surovtseva et al., Characterization of Cardiac Glycoside Natural Products as Potent Inhibitors of DNA Double-Strand Break Repair by a Whole-Cell Double Immunofluorescence Assay. J. Am. Chem. Soc. 138, 3844-3855 (2016). Cells were fixed with 4% paraformaldehyde + 0.02% Triton X-100 in 1X PBS for 20 minutes, washed twice with 1X PBS, and incubated in blocking buffer (10% FBS, 0.5% Triton X-100 in 1X PBS) for 1 h. Rabbit anti-53BP1 antibody (Novus Biologicals, NB100-904) was added 1/1000 in blocking buffer at 4° C. overnight. After washing with 1X PBS, cells were incubated with goat anti-rabbit IgG (H+L) highly cross-adsorbed secondary antibody, Alexa Fluor 647 (Invitrogen, A-21245) 1/500 and with 1 µg/mL Hoechst nucleic acid dye in blocking buffer for 2 h, and then washed with 1X PBS.

pRPA protocol: Cells were washed twice with 1X PBS on ice, incubated in extraction buffer (0.5% Triton X-100 in 1X PBS) for 5 min on ice, fixed with 3% paraformaldehyde + 2% sucrose in 1X PBS for 15 min at 23° C., incubated again in extraction buffer for 5 min on ice, and incubated in blocking buffer (2% BSA, 10% milk, 0.1% Triton X-100 in 1X PBS) for 1 h at 23° C. Rabbit anti-phospho-RPA2 (S33) antibody (Bethyl Laboratories, A300-246A) was added 1/1000 in blocking buffer at 4° C. overnight. After washing 4 times with IF wash buffer (0.1% Triton X-100 in 1X PBS), cells were incubated with goat anti-rabbit IgG (H+L) highly cross-adsorbed secondary antibody, Alexa Fluor 647 (Invitrogen, A-21245) 1/500 and with 1 µg/mL Hoechst nucleic acid dye in blocking buffer for 1 h at 37° C. Cells were washed twice with IF wash buffer and twice with 1X PBS.

Imaging was performed on an InCell Analyzer 2200 Imaging System (GE Corporation) at 40X magnification. Twenty fields-of-view were captured per well. Foci analysis was performed using InCell Analyzer software (GE Corporation). Outer wells were excluded from analysis to limit variation due to edge effects.

Additional small scale immunofluorescence assays used for extended time course analysis of yH2AX foci were performed in Millicell EZ_{SLIDE} 8-well chamber slides (Millipore). Cells were seeded at 10,000 cells/well and allowed to adhere overnight. Following drug treatment, cells were fixed and stained for yH2AX as described above, without the addition of Hoechst dye. Slides were mounted with Vectashield Antifade Mounting Medium with DAPI (Vector Laboratories). Imaging was performed on a Keyence BZ-X800 fluorescence microscope at 40X magnification. Nine adjacent fields-of-view were captured per well and stitched together using a Fiji/ImageJ software plugin. Foci analysis was performed using Focinator v2 software.

Cell Cycle Analysis. Cell cycle analysis was performed using integrated Hoechst nucleic acid dye fluorescence intensity as previously described (51). Briefly, integrated Hoechst fluorescence intensity was log₂ transformed and histograms from DMSO-treated cells were used to identify the centers of the 2 N and 4 N DNA peaks. These values were used to normalize the 2 N DNA peak to 1 and the 4 N DNA peak to 2. Cells were then classified by normalized log₂ DNA content as G1 (0.75-1.25), S (1.25-1.75), or G2 (1.75-2.5) phase cells. The percentage of cells within each phase of the cell cycle was determined for each treatment condition. The three sets of Hoechst-stained cells corresponding to the three separate DNA foci stains were treated as three independent analyses.

Micronuclei Analysis. An automated image analysis pipeline was developed by YCMD using InCell Analyzer software to quantify micronuclei formation. Nuclei and micronuclei were segmented based on Hoechst nucleic acid dye staining channel. A perinuclear margin was applied around the nuclei to approximate the extent of the cytoplasm and identify micronuclei associated with the parent nucleus. Cells with nuclei associated with at least 1 micronucleus were considered positive.

Statistical analysis. Statistical analysis was performed using GraphPad Prism software. Data are presented as mean or median ± SD or SEM as indicated. For in vitro short-term growth delay experiments, IC₅₀ values were determined from the nonlinear regression equation, [inhibitor] vs normalized response with variable slope. For micronuclei assays, comparisons were made with one-way ANOVA and Sidak correction for multiple comparisons. For xenograft growth delay experiments, comparisons were made with Mann-Whitney test (for comparison of 2 groups) or Kruskal-Wallis test with FDR-adjusted p-values with Q set to 5% (for comparison of ≥ 3 groups). For xenograft survival analysis, Kaplan-Meier analysis was used to evaluate survival rate based on death or removal from study when body weight loss exceeded 20% of initial body weight.

### Mouse Protocols

Animals. All animal use was in accordance with the guidelines of the Animal Care and Use Committee (IACUC) of Yale University and conformed to the recommendations in the Guide for the Care and Use of Laboratory Animals (Institute of Laboratory Animal Resources, National Research Council, National Academy of Sciences, 1996).

Mouse Protocols for Flank Studies. A mouse tumor model was established by subcutaneously implanting human LN229 (MGMT-/MMR+) or LN229 (MGMT-/MMR-) cells. Cells were cultured as a monolayer in DMEM +10% FBS (Thermo Fisher) at 37° C. in a humidified atmosphere with 5% CO₂ and passaged between one and three days prior to implantation and media was replaced every 2-3 days as needed to maintain cell viability. Cells were not allowed to exceed 80% confluency. On the day of implantation, cells were trypsinized, washed with complete media and pelleted by centrifugation at 1200 rpm for 5 minutes. The supernatant was decanted, and cells were washed three times with sterile PBS and pelleted by centrifugation. During the final centrifugation, viability was determined using trypan blue exclusion. Cells were resuspended in sterile PBS and diluted 1:1 in Matrigel (Corning, Cat #47743-716) for a final concentration of 5×10⁶ cells/ 100 µL. 5 million cells were injected into the flank of female nude mice (Envigo, Hsd:Athymic Nude-Foxn1^{nu}, 3-4 weeks age, 15 g). Once tumors reached a minimum volume of 100 mm³, mice were randomized and administered either KL-50 (4a; 5 mg/kg MWF × 3 weeks), TMZ (1a; 5 mg/kg MWF × 3 weeks), or vehicle (10% cyclodextrin) by oral gavage. Caliper measurements were obtained during the dosing period and at least two weeks following treatment. Mice were euthanized if body weight loss exceeded 20% or if tumor volume increased to greater than 2000 mm³. Kaplan-Meier analysis was used to evaluate survival rate based on death or removal from study.

In a second study, mice were randomized and administered either KL-50 (4a) or vehicle (10% cyclodextrin) by oral gavage or intraperitoneal injection on either M-F × 1 or MWF × 3 cycles at 5, 15, or 25 mgs/kg. Caliper measurements were obtained during the dosing period and at least two weeks following treatment. Mice were euthanized if body weight loss exceeded 20% or if tumor volume increased to greater than 2000 mm³.

The third study involved MGMT-/MMR+ and MGMT-/MSH6- (shMSH6) LN229 cells. Mice tumors were allowed to grow to a larger average starting volume of ~350 mm³ before they were randomized and administered either KL-50 (4a; 25 mg/kg MWF × 3 weeks) or vehicle (10% cyclodextrin) by oral gavage. Caliper measurements were obtained during the dosing period and at least two weeks following treatment. Mice were euthanized if body weight loss exceeded 20% or if tumor volume increased to greater than 3000 mm³.

Mouse Protocol for Intracranial Study. LN229 MGMT-/MMR- cells stably expressing firefly luciferase (lentivirus-plasmids from Cellomics Technology; PLV-10003), were injected intracranially using a stereotactic injector. Briefly, 1.5 million cells in 5 µl PBS were injected into the brain and the mice were imaged weekly using the IVIS Spectrum In Vivo Imaging System (PerkinElmer) according to the manufacturer’s protocol. Images were taken on a weekly basis and acquired 10 min post intraperitoneal injection with d-luciferin (150 mg/kg of animal mass). Tumors were allowed to grow to an average of 1.0 × 10⁸ RLU before randomization and treated with 5 continuous days of P.O treatment with 10% cyclodextrin vehicle control, TMZ (1a, 25 mg/kg M-F × 1 week) or KL-50 (4a, 25 mg/kg M-F × 1 week). Quantification of BLI flux (photons/sec) was made through the identification of a region of interest (ROI) for each tumor.

Example 1: Synthesis of 2,5-dioxopyrrolidin-1-yl (2-fluoroethyl)carbamate

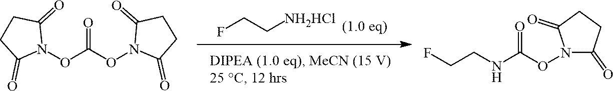

To a mixture of 2-fluoroethylamine hydrochloride (1.94 g, 19.5 mmol, 1.0 eq) in MeCN (25 mL) was added dropwise DIPEA (2.52 g, 19.5 mmol, 3.4 mL, 1.0 eq) at 25° C. The above solution was added dropwise to a solution of bis(2,5-dioxopyrrolidin-1-yl) carbonate (5.00 g, 19.5 mmol, 1.0 eq) in MeCN (50 mL) at 25° C. The reaction mixture was stirred at 25° C. for 12 hrs. Sample was taken from the reaction mixture by dropping tube and diluted with MeCN to 1.0 mL to check the reaction via TLC. TLC (petroleum ether: ethyl acetate = 0: 1, Rf = 0.57) showed the starting material was consumed completely. The reaction mixture was concentrated in vacuum to get a residue. The residue was dissolved in EtOAc (30 mL), and was washed with a saturated aqueous solution of Na₂CO₃ (25 mL × 3). The organic phase was washed with brine (30 mL), dried over Na₂SO₄, and concentrated to afford the title compound (1.00 g, 4.90 mmol, 25.1% yield) as a white solid. ¹H NMR: 400 MHz DMSO-d6 8.56 (t, J = 5.4 Hz, 1H), 4.52 (t, J = 4.8 Hz, 1H), 4.40 (t, J = 4.8 Hz, 1H), 3.39-3.43 (m, 1H), 3.33-3.36 (m, 1H), 2.76 (s, 4H).

Example 2: Synthesis of 3-((4-amino-2-methylpyrimidin-5-yl)methyl)-1-(2-fluoroethyl)-1-nitrosourea

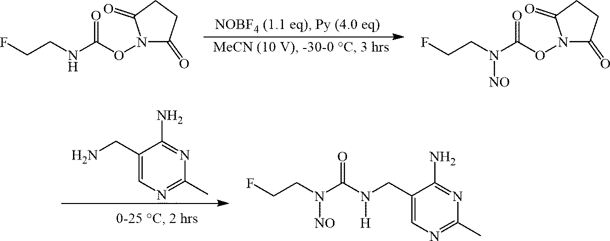

To a solution of compound 2,5-dioxopyrrolidin-1-yl (2-fluoroethyl)carbamate (250 mg, 1.22 mmol, 1.0 eq) in MeCN (2.5 mL) was added pyridine (387 mg, 4.90 mmol, 395 µL, 4.0 eq) and NOBF₄ (157 mg, 1.35 mmol, 1.1 eq) at -30° C. The mixture was stirred at -30° C. for 0.5 hrs. Then the mixture was warmed to 0° C. and stirred at 0° C. for 3.5 hrs. TLC (petroleum ether: ethyl acetate = 2: 3, Rf = 0.64) showed the starting material was consumed completely. To the above reaction was added 5-(aminomethyl)-2-methylpyrimidin-4-amine (168 mg, 1.22 mmol, 1.0 eq) at 0-5° C. and stirred at 25° C. for 2 hrs. TLC (petroleum ether: ethyl acetate = 2: 3, Rf = 0.11) showed the starting material was consumed completely. The reaction mixture was concentrated in vacuum to get a residue. The crude product was purified by prep-HPLC (column: waters Xbridge 150*25 mm*5 um; mobile phase: [water - ACN]; gradient: 13%-43% B over 9 min) to afford the title compound (28.0 mg, 117.08 µmol, 96.1% purity) as a light yellow solid. ¹H NMR: 400 MHz DMSO-d6 9.18 (t, J = 6.0 Hz, 1H), 7.96 (s, 1H), 6.75 (s, 2H), 4.46 (t, J = 4.0 Hz, 1H), 4.34 (t, J = 6.0 Hz, 1H), 4.22 (d, J = 8.0 Hz, 2H), 4.11 (t, J = 4.0 Hz, 1H), 4.05 (t, J = 4.0 Hz, 1H), 2.29 (s, 3H). LC-MS: 257.0 [M+1]⁺.

Example 3: Synthesis of diethyl (1-(3-(2-fluoroethyl)-3-nitrosoureido)ethyl)phosphonate

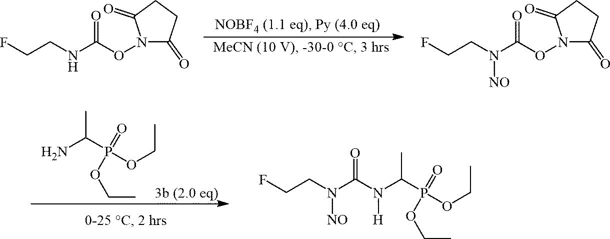

A thumb bottle was equipped with magnetic stirrer, addition funnel and thermometer (-80-55° C.). To a solution of 2,5-dioxopyrrolidin-1-yl (2-fluoroethyl)carbamate (250 mg, 1.22 mmol, 1.0 eq) in MeCN (2.5 mL) was added pyridine (387 mg, 4.90 mmol, 395 µL, 4.0 eq) and NOBF₄ (157 mg, 1.35 mmol, 1.1 eq) at -30° C. The mixture was stirred at -30° C. for 0.5 hrs. Then the mixture was warmed to 0° C. and stirred at 0° C. for 3.5 hrs. TLC (petroleum ether: ethyl acetate = 2: 3, Rf = 0.65) showed the starting material was consumed completely. To the above reaction was added diethyl (1-aminoethyl)phosphonate (443 mg, 2.0 eq) at 0-5° C. and stirred at 25° C. for 2 hrs. TLC (petroleum ether: ethyl acetate = 2: 3, Rf = 0.21) showed the starting material was consumed completely. The reaction mixture was concentrated in vacuum to get a residue. The crude product was purified by prep-HPLC (column: waters Xbridge Prep OBD C18 150*40 mm*10 um; mobile phase: [water - ACN]; gradient: 12% - 42% B over 15 min) to afford the title compound (32.0 mg, 110 µmol, 97.4% purity) as a light yellow solid. ¹H NMR: 400 MHz DMSO-d6 8.89 (d, J = 9.2 Hz, 1H), 4.36-4.47 (m, 1H), 4.25-4.36 (m, 2H), 4.11-4.20 (m, 1H), 4.03-4.06 (m, 5H), 1.36-1.41 (m, 3H), 1.18-1.36 (m, 6H). LC-MS: 300.0 [M+1]⁺.

Example 4: Synthesis of 3-cyclohexyl-l-(2-fluoroethyl)-l-nitrosourea

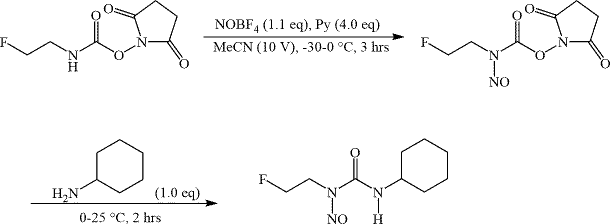

To a solution of 2,5-dioxopyrrolidin-1-yl (2-fluoroethyl)carbamate (250 mg, 1.22 mmol, 1.0 eq) in MeCN (2.5 mL) was added pyridine (387 mg, 4.90 mmol, 395 µL, 4.0 eq) and NOBF₄ (157 mg, 1.35 mmol, 1.1 eq) at -30° C. The mixture was stirred at -30° C. for 0.5 hrs. Then the mixture was warmed to 0° C. and stirred at 0° C. for 2.5 hrs. TLC (petroleum ether: ethyl acetate = 2: 3, Rf = 0.64) showed the starting material was consumed completely. To the above reaction was added cyclohexylamine (121 mg, 1.22 mmol, 140 µL, 1.0 eq) at 0-5° C. and stirred at 25° C. for 2 hrs. TLC (petroleum ether: ethyl acetate = 2: 3, R_{f} = 0.80) showed the starting material was consumed completely. The reaction mixture was concentrated in vacuum to get a residue. The residue was dissolved in MTBE (5.0 mL), and was washed with a saturated aqueous solution of citric acid (4.0 mL × 2). The organic phase was washed with a saturated aqueous solution of NaHCO₃ (5.0 mL × 2), dried over Na₂SO₄, and concentrated to afford the title compound (27.0 mg, 132 µmol, 99.2% purity). ¹H NMR: 400 MHz DMSO-d6 8.52 (d, J = 8.0 Hz, 1H), 4.46 (t, J = 5.0 Hz, 1H), 4.35 (t, J = 5.0 Hz, 1H), 4.12 (d, J = 5.0 Hz, 1H), 4.06 (d, J = 2.6 Hz, 1H), 3.66-3.69 (m, 1H), 1.84-1.85 (m, 2H), 1.81-1.82 (m, 2H), 1.70-1.74 (m, 1H), 1.27-1.42 (m, 4H), 1.11-1.20 (m, 1H).

Example 5: Synthesis of 3-(2-chloroethyl)-1-(2-fluoroethyl)-1-nitrosourea

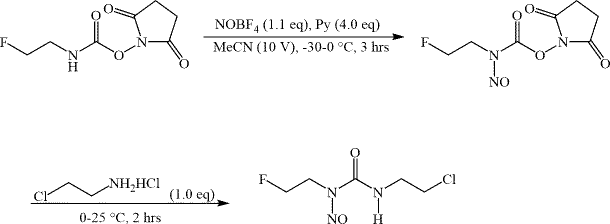

To a solution of 2,5-dioxopyrrolidin-1-yl (2-fluoroethyl)carbamate (250 mg, 1.22 mmol, 1.0 eq) in MeCN (2.5 mL) was added pyridine (387 mg, 4.90 mmol, 395 µL, 4.0 eq) and NOBF₄ (157 mg, 1.35 mmol, 1.1 eq) at -30° C. The mixture was stirred at -30° C. for 0.5 hrs. Then the mixture was warmed to 0° C. and stirred at 0° C. for 2.5 hrs. TLC (petroleum ether: ethyl acetate = 2: 3, Rf = 0.64) showed the starting material was consumed completely. To the above reaction was added 2-chloroethylamine hydrochloride (142 mg, 1.22 mmol, 1.0 eq) at 0-5° C. and stirred at 25° C. for 2 hrs. TLC (petroleum ether: ethyl acetate = 2: 3, R_{f} = 0.78) showed the starting material was consumed completely. The reaction mixture was concentrated in vacuum to get a residue. The residue was dissolved in MTBE (5 mL), and washed with a saturated aqueous solution of citric acid (4 mL × 2). The organic phase was washed with a saturated aqueous solution of NaHCO₃ (5 mL × 2), dried over Na₂SO₄, and concentrated to afford the title compound (30.0 mg, 156 µmol, 96.4% purity). ¹H NMR: 400 MHz DMSO-d6 8.97 (t, J = 5.0 Hz, 1H), 4.48 (t, J = 2.6 Hz, 1H), 4.36 (t, J = 5.2 Hz, 1H), 4.11 (t, J = 6.8 Hz, 1H), 4.07 (t, J = 2.4 Hz, 1H), 3.75-3.78 (m, 1H), 3.62-3.64 (m, 1H).

Example 6: Synthesis of KL-50 (4a):

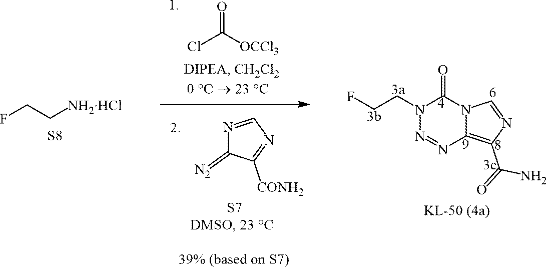

A mixture of fluoroethylamine hydrochloride (3.32 g, 33.3 mmol, 1 equiv), and N,N-di-iso-propyl ethylamine (12.2 mL, 70.0 mmol, 2.10 equiv) in dichloromethane (80 mL) was added dropwise via syringe pump over 45 min to a solution of diphosgene (2.40 mL, 20.0 mmol, 0.60 equiv) in dichloromethane (80 mL) at 0° C. Upon completion of the addition, the cooling bath was removed, and the reaction mixture was allowed to warm to 23° C. over a period of 15 min. The warmed product mixture was immediately transferred to a separatory funnel. The organic layer was washed sequentially with 1 N aqueous hydrochloric acid solution (100 mL, precooled to 0° C.) and saturated aqueous sodium chloride solution (100 mL, precooled to 0° C.). The washed organic layer was dried over magnesium sulfate. The dried solution was filtered, and the filtrate was concentrated (330 mTorr, 31° C.). The unpurified isocyanate so obtained was used directly in the following step.

The unpurified isocyanate obtained in the preceding step (nominally 16.7 mmol, 1.75 equiv) was added dropwise via syringe to a solution of the diazonium S7 (1.31 g, 9.54 mmol, 1 equiv) in dimethyl sulfoxide (10 mL) at 23° C. Upon completion of the addition, the reaction vessel was covered with aluminum foil. The reaction mixture was stirred for 16 h at 23° C. The product mixture was concentrated under a stream of nitrogen. The residue obtained was suspended in dichloromethane and purified by automated flash-column chromatography (eluting with 100% dichloromethane initially, grading to 5% methanol-dichloromethane, linear gradient) to provide KL-50 (4a) as a white crystalline powder (840 mg, 39% based on the diazonium S7). ¹H NMR (400 MHz, DMSO-d₆) δ 8.85 (s, 1H, H₆), 7.83 (s, 1H, NH), 7.70 (s, 1H, NH), 4.82 (dt, J = 47.0, 4.9 Hz, 2H, H_{3b}), 4.62 (dt, J = 26.0, 4.7 Hz, 2H, H₃ₐ). ¹³C NMR (151 MHz, DMSO-d₆) δ 161.5 (C₈ₐ), 139.2 (C₄), 134.2 (C₉), 131.0 (C₈), 128.9 (C₆), 80.8 (d, J = 168.7 Hz, C_{3b}), 49.1 (d, J = 20.8 Hz, C₃ₐ). ¹⁹F NMR (376 MHz, DMSO-d₆) δ -222.66 (tt, J = 47.0, 26.1 Hz). IR (ATR-FTIR), cm⁻¹: 3459 (w), 3119 (m), 1736 (s), 1675 (s). HRMS-ESI (m/z): [M + H]⁺ calcd for [C₇H₈FN₆O₂]⁺ 227.0688, found 227.0676.

Synthesis of the imidazolyl triazene 10:

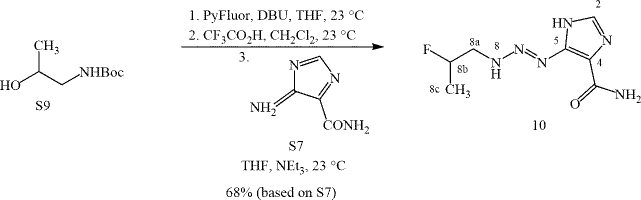

Tert-butyl (2-hydroxypropyl)carbamate (1.72 mL, 10.0 mmol, 1 equiv) was added dropwise via syringe to a mixture of PyFluor (1.77 g, 11.0 mmol, 1.10 equiv) in tetrahydrofuran (10 mL) at 23° C. 1,8-Diazabicyclo(5.4.0)undec-7-ene (3.00 mL, 20.0 mmol, 2.00 equiv) was immediately added dropwise and the reaction mixture was stirred for 48 h at 23° C. under ambient atmosphere. The product mixture was diluted with water (15 mL) and the resulting biphasic mixture was transferred to a separatory funnel. The layers that formed were separated and the aqueous layer was extracted with ethyl acetate (2 × 15 mL). The organic layers were combined and the combined organic layer was washed sequentially with 1 N aqueous hydrochloric acid solution (2 × 25 mL) and saturated aqueous sodium chloride solution (2 × 25 mL). The washed organic layer was dried over sodium sulfate. The dried solution was then filtered and the filtrate concentrated to provide tert-butyl (2-fluoropropyl)carbamate as a clear colorless oil.

The unpurified product obtained in the preceding step (nominally 6 mmol, 1 equiv) was added to a mixture of dichloromethane (30 mL) and trifluoroacetic acid (10 mL) at 23° C. The reaction mixture was stirred for 12 h at 23° C. under ambient atmosphere. The product mixture was concentrated to provide 2-fluoropropylamine trifluoroacetic acid as an opaque oil with excess equivalents of trifluoroacetic acid. The unpurified product obtained in this way (nominally 6 mmol) was dissolved in tetrahydrofuran (10 mL) to generate a working nominal 0.6 M solution for future reactions.

A solution of 2-fluoropropylamine trifluoroacetic acid in tetrahydrofuran (4.40 mL, 2.64 mmol, 1.05 equiv) and triethylamine (1.40 mL, 10 mmol, 4.00 equiv) were added sequentially dropwise via syringe to a suspension of the diazonium S7 (343 mg, 2.50 mmol, 1 equiv) in tetrahydrofuran (15 mL) at 23° C. The reaction mixture was stirred for 6 h at 23° C. The precipitate that formed was collected by vacuum filtration. The precipitate was washed sequentially with ethyl acetate (2 × 15 mL) and diethyl ether (2 × 15 mL). The washed precipitate was dried in vacuo to afford the imidazolyl triazene 10 as a light tan powder (365 mg, 68%, based on the diazonium S7). ¹H NMR (600 MHz, DMSO-d₆) δ 12.65 (s, 1H, NH), 10.92 (s, 1H, H₈), 7.54 (s, 1H, H₂), 7.45 (br s, 1H, NH), 7.21 (s, 1H, NH), 4.98 (br d, J= 49 Hz, 1H, H_{8b}), 3.87 - 3.55 (m, 2H, H₈ₐ), 1.34 (dd, J= 23.9, 6.3 Hz, 3H, H_{8c}). ¹³C NMR (151 MHz, DMSO-d₆) δ 161.0 (C_{quat.}), 149.5 - 148.9 (br s, C_{quat.})^{a}, 135.6 (C₂), 115.9 (C_{quat.}), 87.1 (d, J = 166.6 Hz, C_{8b}), 48.1 (d, J = 22.3 Hz, C₈ₐ), 18.8 (d, J= 21.7 Hz, C_{8c}). ¹⁹F NMR (376 MHz, DMSO-d₆) δ -174.43 (dq, J = 47.7, 23.9 Hz). IR (ATR-FTIR), cm⁻¹: 3480 (w), 3249 (m), 3077 (m), 1638 (s), 1590 (s), 1427 (s), 1397 (s). HRMS-ESI (m/z): [M + H]⁺ calcd for [C₇H₁₂FN₆O]⁺ 215.1052, found 215.1048. Note: ^{a}Broad peak tentatively attributed to a quaternary carbon from the imidazolyl triazene 10.

Synthesis of imidazolyl triazene 11:

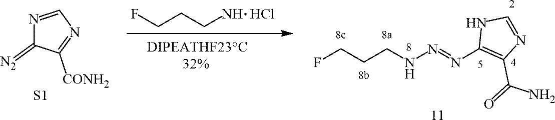

N,N-Di-iso-propyl ethylamine (834 µL, 4.55 mmol, 1.25 equiv) was added dropwise via syringe to a mixture of (3-fluoropropyl)amine hydrochloride (410 mg, 3.65 mmol, 1 equiv) and the diazonium S7 (500 mg, 3.65 mmol, 1 equiv) in tetrahydrofuran (25 mL) at 23° C. The reaction mixture was stirred for 6 h at 23° C. The precipitate that formed was collected by vacuum filtration. The precipitate was washed sequentially with ethyl acetate (2 × 15 mL) and ether (2 × 15 mL). The washed precipitate was dried in vacuo to afford the imidazolyl triazene 11 as a light tan powder (251 mg, 32%). ¹H NMR (400 MHz, DMSO-d₆) δ 12.63 (s, 1H, NH), 10.72 (s, 1H, H₈), 7.54 (s, 1H, H₂), 7.46 (s, 1H, NH), 7.28 (s, 1H, NH), 4.54 (dt, J = 47.3, 5.8 Hz, 2H, H_{8c}), 3.60 - 3.50 (m, 2H, H_{8b}), 2.08 - 1.94 (m, 2H, H_{8af}). ¹³C NMR (151 MHz, DMSO-d₆) δ 161.3 (Cquat.), 155.4 (Cquat.), 149.7 (Cquat), 135.9 (C₂)^{a}, 81.9 (d, J = 161.4 Hz, C_{8c}), 39.6 (C_{8b})^{b}, 26.7 (d, J = 19.9 Hz, C₈ₐ). ¹⁹F NMR (376 MHz, DMSO-d₆) δ -219.23 (tt, J= 47.1, 26.1 Hz). IR (ATR-FTIR), cm⁻¹: 3483 (w), 3269 (m), 3082 (m), 1640 (m), 1587 (m), 1392 (m). HRMS-ESI (m/z): [M + Na]⁺ calcd for [C₇H₁₁FN₆NaO]⁺ 237.0871, found 237.0986.

### Example 7: Polymorph of KL-50

Single crystals of KL-50 (4a) suitable for X-ray analysis were obtained by vapor diffusion of dry benzene (3 mL, precipitating solvent) into a syringe filtered (Millipore Sigma, 0.22 µm, hydrophilic polyvinylidene fluoride, 33 mm, gamma sterilized, catalogue number SLGV033RS) solution of KL-50 (4a) (3.6 mg) in dry dichloromethane (3 mL, solubilizing solvent) at 23° C. This yielded two polymorphs of KL-50 (4a) designated Polymorph I (P2₁/n space group, CCDC number 2122008) and Polymorph II (Cc space group, CCDC number 2122009).

### Experimental Procedure for Polymorph I of KL-50 (4a):

Low-temperature diffraction data (ω-scans) were collected on a Rigaku MicroMax-007HF diffractometer coupled to a Dectris Pilatus3R detector with Mo Kα (λ = 0.71073 Å) for the structure of 007c-21083. The diffraction images were processed and scaled using Rigaku Oxford Diffraction software (CrysAlisPro; Rigaku OD: The Woodlands, TX, 2015). The structure was solved with SHELXT and was refined against F² on all data by full-matrix least squares with SHELXL (Sheldrick, G. M. Acta Cryst. 2008, A64, 112-122). All non-hydrogen atoms were refined anisotropically. Hydrogen atoms were included in the model at geometrically calculated positions and refined using a riding model. The isotropic displacement parameters of all hydrogen atoms were fixed to 1.2 times the U value of the atoms to which they are linked (1.5 times for methyl groups).

TABLE 2

Crystal data and structure refinement for Polymorph I of KL-50 (4a)

| | |
|---|---|
| Identification code | 007c-21083 |
| Empirical formula | C7 H7 F N6 02 |
| Formula weight | 226.19 |
| Temperature | 93(2) K |
| Wavelength | 0.71073 Å |
| Crystal system | Monoclinic |
| Space group | P2₁/n |
| Unit cell dimensions | a = 7.1861(5) Å |
| | b = 7.6918(5) Å |
| | c = 16.4546(12) Å |
| Volume | 903.19(11) Å³ |
| Z | 4 |
| Density (calculated) | 1.663 Mg/m³ |
| Absorption coefficient | 0.141 mm⁻¹ |
| F(000) | 464 |
| Crystal size | 0.200 × 0.200 × 0.020 mm³ |
| Crystal color and habit | Colorless Plate |
| Diffractometer | Dectris Pilatus 3R |
| Theta range for data collection | 2.927 to 31.467°. |
| Index ranges | -9<=h<=10, -10<=k<=9, -23<=1<=19 |
| Reflections collected | 9836 |
| Independent reflections | 2539 [R(int) = 0.0300] |
| Observed reflections (I > 2sigma(I)) | 2135 |
| Completeness to theta = 25.242° | 99.9% |
| Absorption correction | Semi-empirical from equivalents |
| Max. and min. transmission | 1.00000 and 0.31047 |
| Solution method | SHELXT-2014/5 (Sheldrick, 2014) |
| Refinement method | SHELXL-2014/7 (Sheldrick, 2014) |
| Data / restraints / parameters | 2539/0/145 |
| Goodness-of-fit on F² | 1.050 |
| Final R indices [I>2sigma(I)] | R1 = 0.0358, wR2 = 0.0885 |
| R indices (all data) | R1 = 0.0450, wR2 = 0.0929 |
| Largest diff. peak and hole | 0.360 and -0.250 e.Å⁻³ |

### Example 8: Polymorph II of KL-50

Polymorph II of KL-50 was prepared and characterized.

### Experimental Procedure for Polymorph II of KL-50 (4α):

Low-temperature diffraction data (ω-scans) were collected on a Rigaku MicroMax-007HF diffractometer coupled to a Saturn994+ CCD detector with Cu Kα (λ = 1.54178 Å) for the structure of 007b-21124. The diffraction images were processed and scaled using Rigaku Oxford Diffraction software (CrysAlisPro; Rigaku OD: The Woodlands, TX, 2015). The structure was solved with SHELXT and was refined against F² on all data by full-matrix least squares with SHELXL (Sheldrick, G. M. Acta Cryst. 2008, A64, 112-122). All non-hydrogen atoms were refined anisotropically. Hydrogen atoms were included in the model at geometrically calculated positions and refined using a riding model. The isotropic displacement parameters of all hydrogen atoms were fixed to 1.2 times the U value of the atoms to which they are linked (1.5 times for methyl groups).

TABLE 3

Crystal data and structure refinement for Polymorph II of KL-50 (4a)

| Identification code | 007b-21124 |
|---|---|
| Empirical formula | C7 H7 F N6 02 |
| Formula weight | 226.19 |
| Temperature | 93(2) K |
| Wavelength | 1.54184 Å |
| Crystal system | Monoclinic |
| Space group | Cc |
| Unit cell dimensions | a = 6.6061(2) Å |
| | b = 23.1652(6) Å |
| | c = 11.9879(3) Å |
| Volume | 1824.52(9) Å³ |
| Z | 8 |
| Density (calculated) | 1.647 Mg/m³ |
| Absorption coefficient | 1.218 mm⁻¹ |
| F(000) | 928 |
| Crystal size | 0.200 × 0.020 × 0.020 mm³ |
| Crystal color and habit | Colorless Needle |
| Diffractometer | Rigaku Saturn 944+ CCD |
| Theta range for data collection | 3.816 to 66.573°. |
| Index ranges | -7<=h<=7, -27<=k<=27, -14<=1<=14 |
| Reflections collected | 31349 |
| Independent reflections | 3204 [R(int) = 0.0818] |
| Observed reflections (I > 2sigma(I)) | 2950 |
| Completeness to theta = 66.573° | 100.0 % |
| Absorption correction | Semi-empirical from equivalents |
| Max. and min. transmission | 1.00000 and 0.59718 |
| Solution method | SHELXT-2014/5 (Sheldrick, 2014) |
| Refinement method | SHELXL-2014/7 (Sheldrick, 2014) |
| Data / restraints / parameters | 3204/2/291 |
| Goodness-of-fit on F² | 1.041 |
| Final R indices [I>2sigma(I)] | R1 = 0.0330, wR2 = 0.0770 |
| R indices (all data) | R1= 0.0381, wR2 = 0.0795 |
| Absolute structure parameter | 0.1(2) |

### Example 9: Synthesis of Additional Compounds

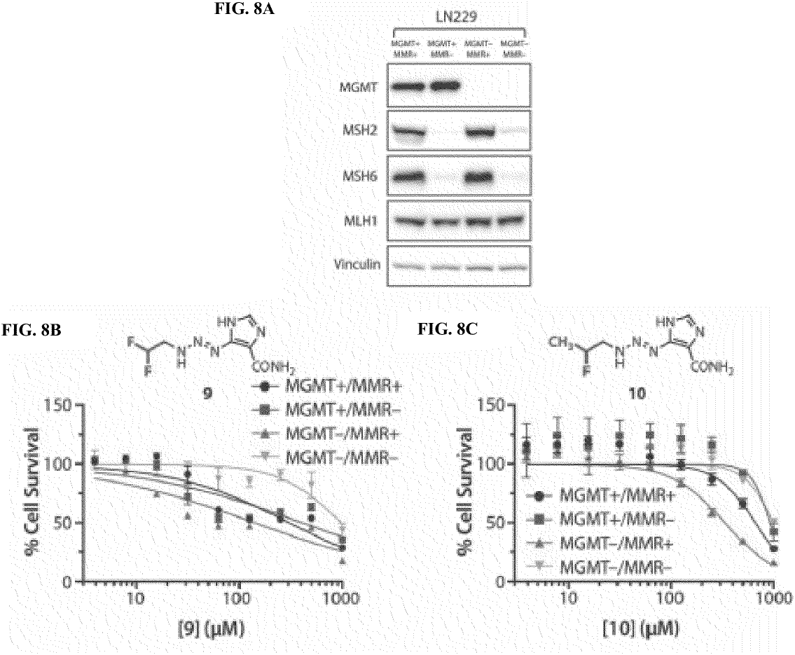

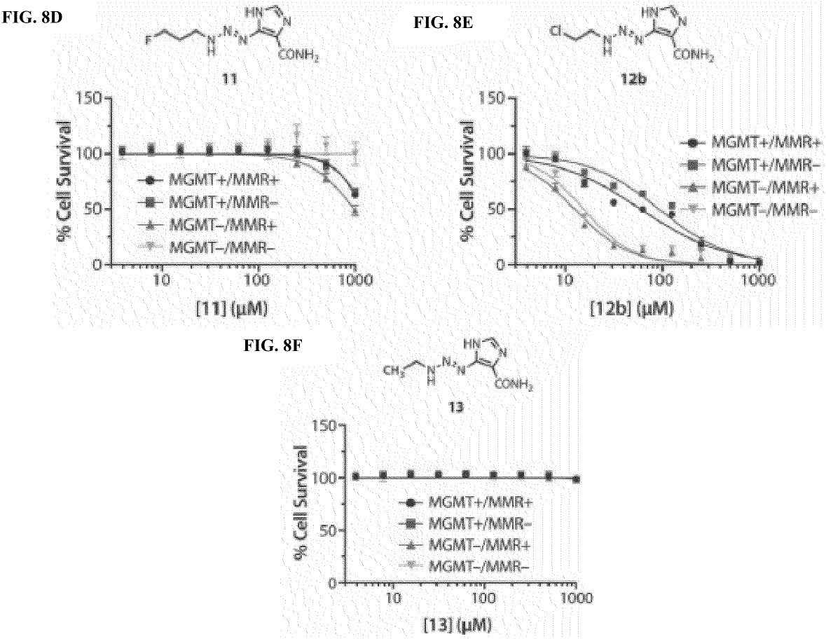

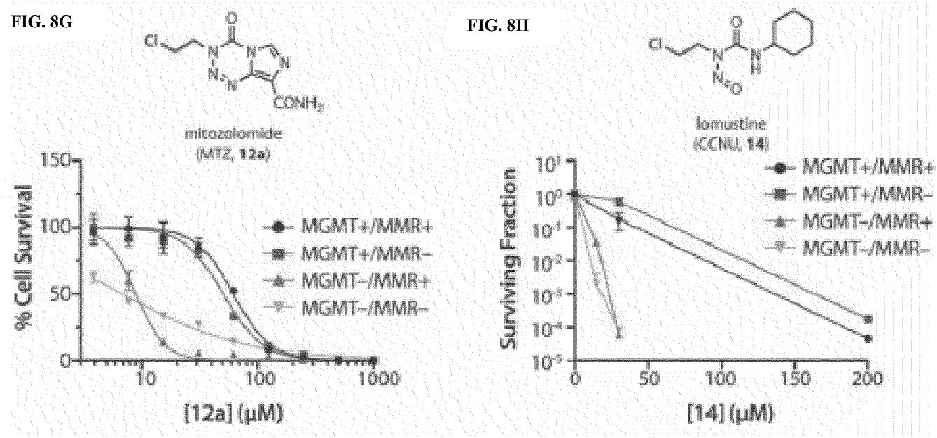

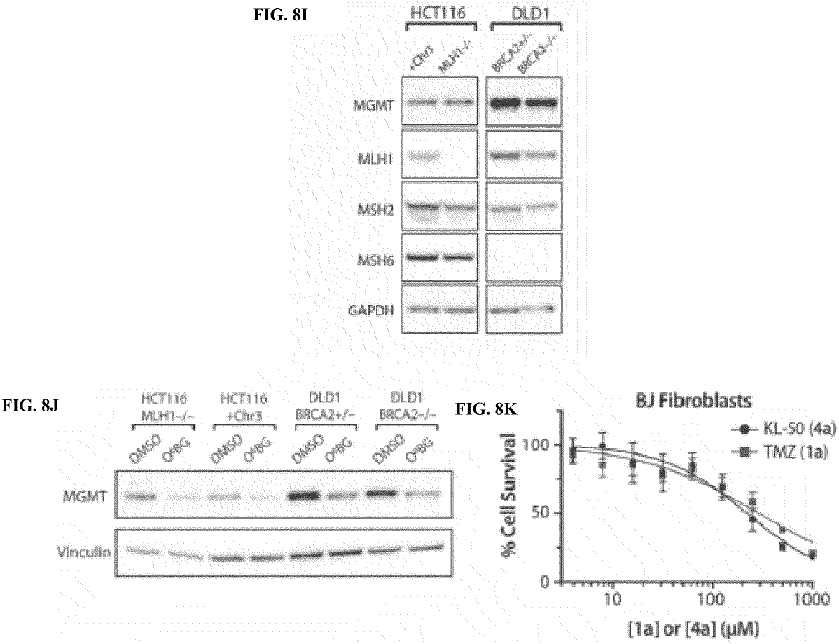

The imidazotetrazine 4a (KL-50) and the triazene 4b (KL-85) were synthesized as vehicles to deliver 2-fluoroethyl diazonium (4c), and a series of related agents to probe structure-activity relationships in tissue culture (FIGS. 1, D and F). The 2-fluoropropyl- and 3-fluoropropyl-triazenes (10 and 11, respectively) were prepared by diazotization of 4-aminoimdazole-5-carboxamide, followed by the addition of the respective amine. All other triazenes were prepared according to literature procedures. The cytotoxicity the compounds described herein was assessed in short-term cell viability assays against four isogenic LN229 glioblastoma cell lines engineered to be proficient or deficient in MGMT and/or MMR activity, using short hairpin RNAs (shRNAs) targeting MSH2 (referred to as MGMT+/-, MMR+/- cells hereafter; FIG. 8A).

### Example 10: Activity Analysis of Compounds

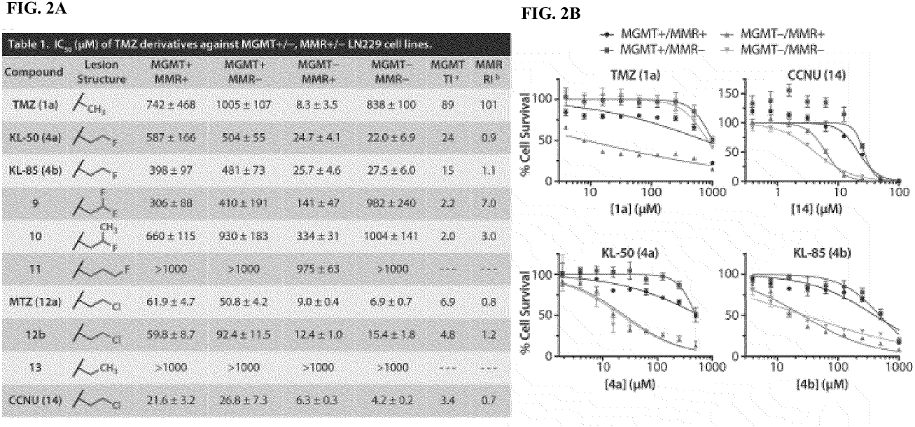

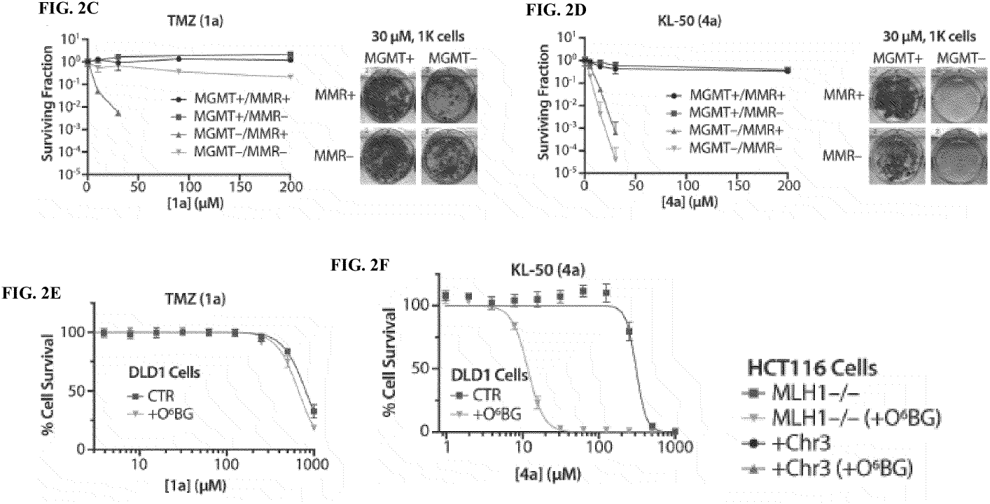

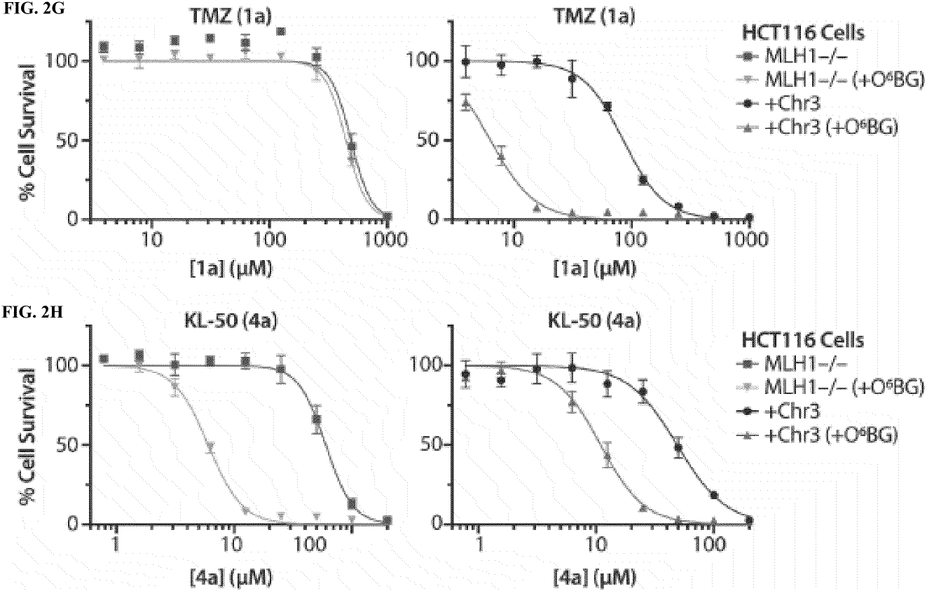

The IC₅₀ values of these agents are shown in Table 1 (FIG. 2A) and representative dose-response curves are shown in FIG. 2B (additional data are presented in FIGS. 8B to 8G). KL-85 (4b) retained potency in MGMT-/MMR- cells (IC₅₀ = 27.5 µM), while TMZ (1a) was essentially inactive (IC₅₀ = 837.7 µM). Structure-activity studies were consistent with the mechanistic pathway shown in FIG. 1E. The 2,2-difluoroethyl triazene 9 and the 2-fluoropropyl triazene 10 possessed reduced potency in MGMT-/MMR- cells (FIGS. 8B and 8C), in agreement with the reduced rates of displacement following introduction of an additional fluorine or alkyl substituent. The 2-chloroethyl triazene 12b was modestly potent but not as selective for MGMT- cell lines (FIG. 8E) which likely derives from faster, non-selective ICL formation arising from chloride displacement (vide infra). The 3-fluoropropyl triazene 11 demonstrated low activity in all four cell lines, presumably due to inefficient transfer of the electrophile to DNA (FIG. 8D). The ethyl triazene 13 also demonstrated low activity (FIG. 8F). This compound may undergo rapid elimination to ethylene gas following conversion to ethyl diazonium.

KL-50 (4a) was prepared by diazotization of 4-aminoimidazole-5-carboxamide followed by the addition of (2-fluoroethyl)isocyanate (39% overall yield, see the Supplementary Information). The potency of 4a mirrored that of 4b in the four cell lines examined (FIG. 2B). To benchmark selectivity, the experimental agent mitozolomide (MTZ, 12a) and the clinical nitrosourea lomustine (CCNU, 14) were evaluated, which have been studied with hopes of addressing TMZ (1a) resistance. However, these agents were only ~4-7-fold selective for MGMT-deficient cells (FIG. 8G and FIG. 2B, respectively), as opposed to the ~25-fold selectivity seen with KL-50 (4a).

### Example 11: Activity Analysis of Compounds

The antitumor activity of KL-50 (4a) was validated in clonogenic survival assays (CSAs) and additional cell lines in vitro. TMZ (1a) possessed negligible activity in MGMT+ LN229 cells, irrespective of MMR status, and induced robust tumor cell killing in MGMT-, MMR+ cells that was abolished in isogenic cells lacking MMR (FIG. 2C). Lomustine (14) was effective in MMR- cells but was cytotoxic to MGMT+ cells (FIG. 8H). In contrast, KL-50 (4a) demonstrated robust antitumor activity in MGMT- cells, independent of MMR status, with minimal toxicity to MGMT+ cells at doses up to at least 200 µM (FIG. 2D). A similar pattern of activities was observed in several unique cell lines across different tumor types with intrinsic or induced loss of MGMT and/or MMR activity. For example, TMZ (1a) was inactive in DLD1 cells, which possess MGMT but lack functional MMR (MSH6-) with or without induced depletion of MGMT using O⁶-benzylguanine (O⁶BG; FIG. 2E). In contrast, KL-50 (4a) was toxic to these cells, but only after O⁶BG-induced MGMT depletion (FIG. 2F). TMZ (1a) was inactive in HCT116 colorectal cancer cells, which lack the MMR protein MLH1, regardless of MGMT levels (FIG. 2G). Restoration of MMR activity via complementation with chromosome 3 containing MLH1 resulted in the enhanced sensitivity to TMZ (1a), which was further potentiated by MGMT depletion (FIG. 2G). In contrast, KL-50 (4a) induced selective tumor cell killing specifically in the setting of O⁶BG-induced MGMT suppression, in both MLH1-deficient and MLH1-complemented cells (FIG. 2H). MMR status and O⁶BG-induced loss of MGMT expression was confirmed by western blot analysis (FIGS. 8A, 8I, and 8J). The activity of KL-50 (4a) was confirmed in MGMT- LN229 cells engineered to lack expression of other key MMR proteins including MSH6, MLH1, PMS2, and MSH3 (FIGS. 9A-9J). Finally, the cytotoxicity of KL-50 (4a) and TMZ (1a) were compared in normal human fibroblast cells and observed no increase in toxicity with KL-50 (4a) (FIG. 8K). These data define KL-50 (4a) as a first-in-class molecule that overcomes MMR mutation-induced resistance while retaining selectivity for tumor cells lacking MGMT.

### Example 12: Activity Analysis of Compounds

A well-established comet assay was used and adapted for ICL detection to determine if ICLs were formed in MGMT- cells treated with KL-50 (4a) (FIGS. 3, A and B). In this assay, cells were sequentially exposed to genotoxins and ionizing radiation, and then analyzed by single cell alkaline gel electrophoresis. Attenuation of the IR-induced comet tail is indicative of ICL formation. In the absence of IR, TMZ (1a, 200 µM) and KL-50 (4a, 200 µM) both induced tailing in MGMT-/MMR+ cells, while mitomycin C (MMC, 0.1 or 50 µM) did not. Exposure to 50 µM MMC for 2 h completely abolished the IR-induced comet tail, whereas exposure to 0.1 µM MMC (chosen to be ~10-fold greater than the IC₅₀ for this drug, comparable to 200 µM KL-50 (4a) or TMZ (1a)) for 24 h caused a partial reduction in the IR-induced comet tail. TMZ (1a, 200 µM) did not reduce DNA migration following IR, in agreement with its known function as a monoalkylation agent with no known crosslinking activity. In contrast, KL-50 (4a, 200 µM) reduced the %DNA in the tail to levels similar to those seen for 0.1 µM MMC. A similar pattern of comet tail migration for MMC and KL-50 (4a) was observed in MGMT-/MMR- cells, which supports an MMR-independent crosslinking mechanism. Comparable results were observed in MGMT-/MMR+ cells treated with KL-85 (4b) (FIGS. 10A and 10B).

This assay was carried out at varying time points (2-24 h) to assess the rates of ICL formation in MGMT-/MMR- cells treated with KL-50 (4a), MTZ (12a), or TMZ (1a) (FIGS. 3, C and D). The chloroethyl derivative MTZ (12a) reduced DNA mobility within 2 h, consistent with the cell line selectivities above and literature reports that this agent rapidly forms ICLs by chloride displacement from other sites of alkylation. TMZ (1a) did not induce a statistically significant decrease in DNA migration within 24 h. However, a time-dependent decrease in DNA mobility was observed in cells treated with KL-50 (4a), with the largest difference observed between 8 and 24 h, consistent with the reported half-life of O⁶FEtG (6; 18.5 h). In the unirradiated samples, KL-50 (4a), MTZ (12a), and TMZ (1a) all induced maximal damage at 2 h, which decreased over time, consistent with progressive DNA repair (FIGS. 10C and 10D). Analysis of genomic DNA isolated from LN229 MGMT-/MMR+ cells treated with KL-50 (4a, 200 µM) or KL-85 (4b, 200 µM) by denaturing gel electrophoresis demonstrated the presence of crosslinked DNA (FIG. 3E). TMZ (1a) and MTIC (1b) showed no evidence of ICL induction. Similarly, linearized pUC19 plasmid DNA treated with KL-50 (4a, 100 µM) also possessed ICLs, with delayed rates of formation relative to 12b (FIG. 3F). Collectively, these data support a mechanism of action for KL-50 (4a) involving the slow generation of DNA ICLs in the absence of MGMT.

### Example 13: Activity Analysis of Compounds

Alternative mechanisms of action implicating nucleotide excision repair (NER), base excision repair (BER), reactive oxygen species (ROS), and DNA duplex destabilization were investigated. Short term cell viability assays in isogenic mouse embryonic fibroblasts (MEFs) proficient or deficient in XPA, a common shared NER factor, revealed no differential sensitivity, either with or without O⁶BG-induced MGMT depletion (FIG. 11A). N7MeG lesions induced by TMZ (1a) are prone to spontaneous depurination, apurinic (AP) site formation, and single strand breaks (SSBs), which are all known BER substrates. To probe for potential differential induction of BER substrates by KL-50 (4a) compared to TMZ (1a), in vitro supercoiled plasmid DNA assays were performed that measure the formation of AP sites. Similar levels of spontaneous and enzyme-catalyzed SSBs were observed from AP sites with KL-50 (4a) and TMZ (1a), suggesting comparable levels of depurination (FIG. 11B). Co-treatment with increasing concentrations of the ROS scavenger N-acetylcysteine (NAC) did not rescue cell viability (FIG. 11C). Melting point analysis did not reveal any notable differences in DNA stability resulting from fluoroethylation compared to methylation (FIG. 11D). These data suggest that NER status, AP site induction, ROS, and altered DNA stability are peripheral or noncontributory to the effectiveness of KL-50 (4a).

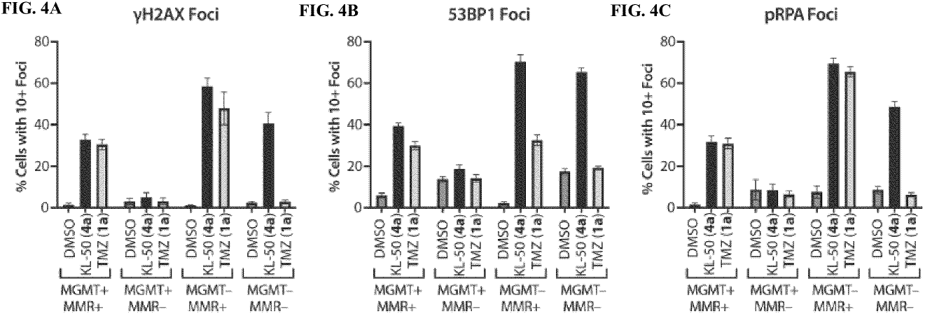

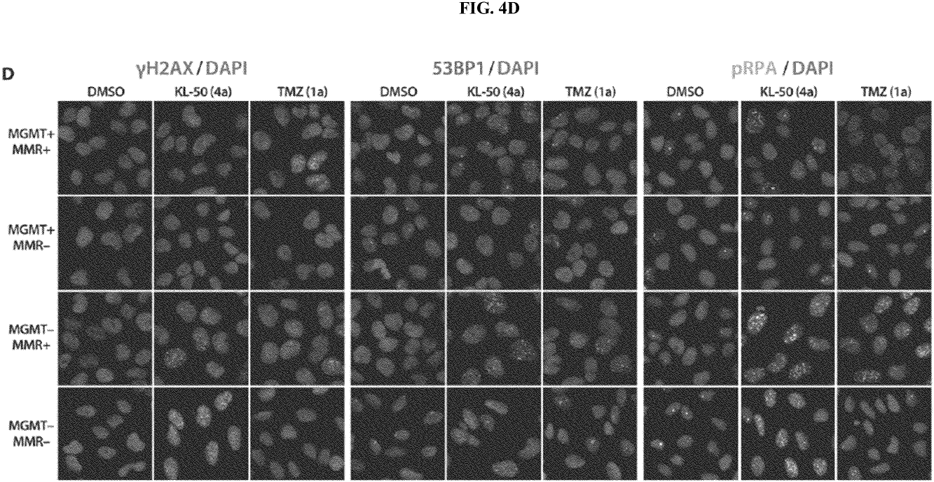

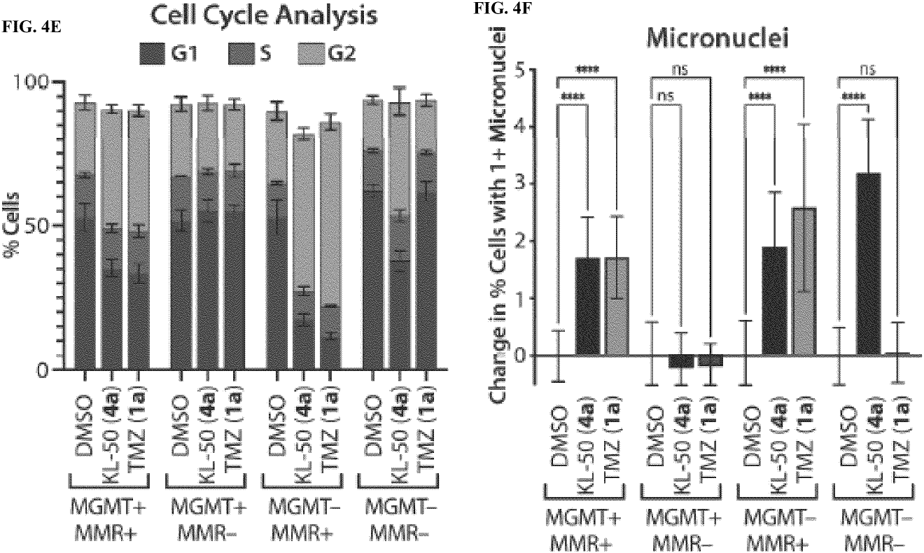

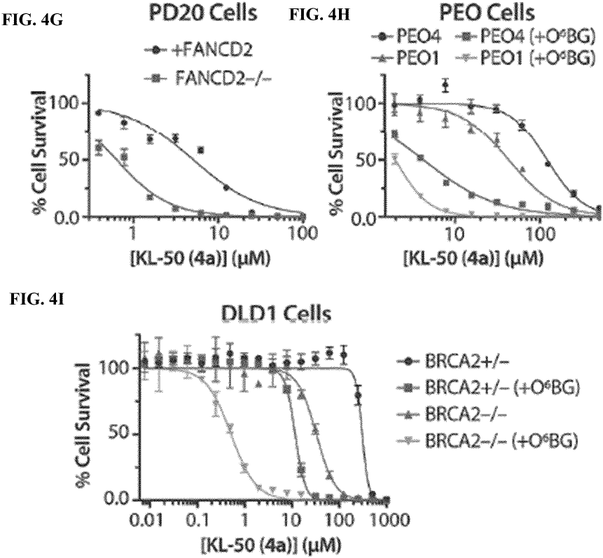

The profile of DDR activation was characterized across four isogenic cell lines after treatment with KL-50 (4a) or TMZ (1a). The prior finding that the ATR-CHK1 signaling axis is activated in response to TMZ (1a)-induced replication stress in MGMT-deficient cells prompted the analysis of the phosphorylation status of CHK1 and CHK2 in LN229 MGMT+/- and MMR+/- cells. KL-50 (4a) induced CHK1 and CHK2 phosphorylation in MGMT- cells regardless of MMR status, whereas TMZ (1a) only induced phospho-CHK1 and -CHK2 in MGMT-/MMR+ cells (FIG. 12A). Foc formation of the DDR factors phospho-SER139-H2AX (yH2AX), p53 binding protein 1 (53BP1), and phospho-SER33-RPA2 (pRPA) was analyzed over the period of 2 to 48 h (FIGS. 4A to 4D, and FIGS. 12B and 12C). KL-50 (4a) induced a maximal foci response at 48 h, specifically in MGMT- cells and irrespective of MMR status (4a). TMZ (1a) induced a comparable response in MGMT- cells, but this was abolished in the absence of functional MMR, consistent with known MMR-silencing-based resistance. A reduced level of foci formation was observed in MGMT+/MMR+ cells that was absent in MGMT+/MMR- cells, suggesting an MMR-dependent DNA damage response in these cells. However, these foci dissipate at later timepoints (72-96 h; FIG. 12D), and they are not associated with appreciable cellular toxicity (as shown earlier in FIGS. 2, C and D).

KL-50 (4a) induced increasing G2 arrest on progression from 24 to 48 h in MGMT-/MMR+ cells, as determined by simultaneous analysis of DNA content based on nuclear (Hoechst) staining in the foci studies above (FIG. 4E and FIGS. 13A and 13B). KL-50 (4a) induced an attenuated G2 arrest in MGMT-/MMR- cells, consistent with a role of MMR in the G2-checkpoint. This effect in MGMT-/MMR- cells was absent following TMZ (1a) treatment. Both TMZ (1a) and KL-50 (4a) induced a moderate G2 arrest in MGMT+/MMR+ cells.

The levels of DDR foci were quantified across the individual cell cycle phases (FIGS. 14A-14F). KL-50 (4a) induced foci formation primarily in the S- and G2-phases of the cell cycle, which is consistent with replication blocking by ICLs. Foci increased in MGMT- G1 cells at 48 h, suggesting that a fraction of cells may progress through S-phase with unrepaired DNA damage. Consistent with this, a significant increase in micronuclei was observed at 48 h following KL-50 (4a) treatment, which was greatest in the MGMT-/MMR- cells (FIG. 4F and FIGS. 15A and 15B). TMZ (1a) displayed a similar pattern of foci induction in the S- and G2-phases, with smaller increases in G1-phase foci and micronuclei formation at 48 h in MGMT-/MMR+ cells. In contrast, foci induction or micronuclei formation was not observed in MGMT-/MMR- cells exposed to TMZ (1a). These findings are in agreement with the differential toxicity profiles of KL-50 (4a) and TMZ (1a): KL-50 (4a) induces multiple successive markers of DNA damage and engagement of the DDR in MGMT- cells, independent of MMR status, whereas the effects of TMZ (1a) are similar in MGMT-/MMR+ cells but absent in MMR- cells. Coupled with the ICL kinetics data presented above, these time-course data support a slow rate of ICL induction in situ by KL-50 (4a).

These foci data suggest that KL-50 (4a) induces replication stress (e.g., pRPA foci formation) and DSB formation (e.g., γH2AX and 53BP1 foci, which are known to follow the formation of ICLs. Consistent with this, BRCA2- and FANCD2-deficient cells are hypersensitive to KL-50 (4a; FIGS. 4, G to I, and FIGS. 15, 15C to 15F). In two MGMT-proficient cell models, BRCA2 loss enhanced the toxicity of KL-50 (4a) following MGMT depletion via O⁶BG (FIGS. 4H and 4I). FANCD2 ubiquitination by KL-50 (4a) was observed specifically in MGMT- cells, suggesting activation of the Fanconi anemia (FA) ICL repair pathway (FIG. 15G). As previously reported, TMZ (1a) also induced FANCD2 ubiquitination but only in MGMT-/MMR+ cells.

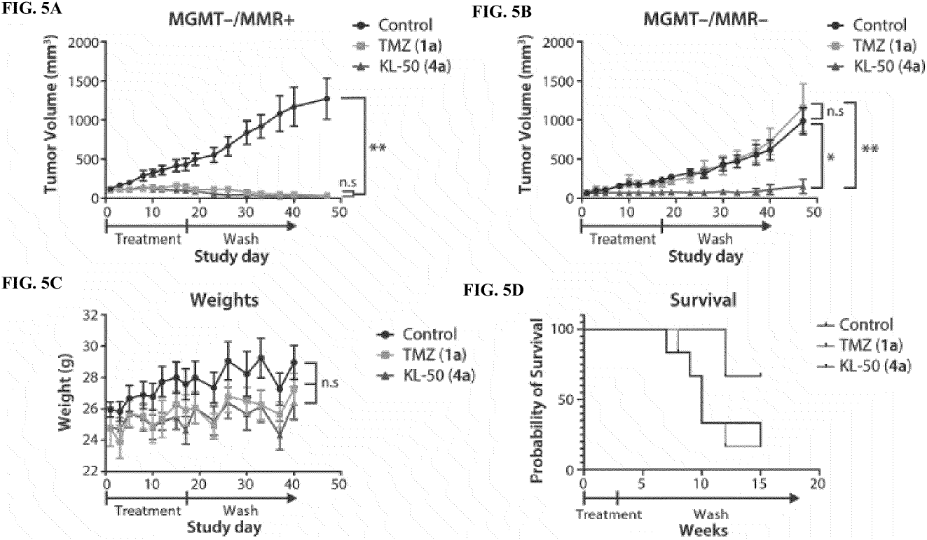

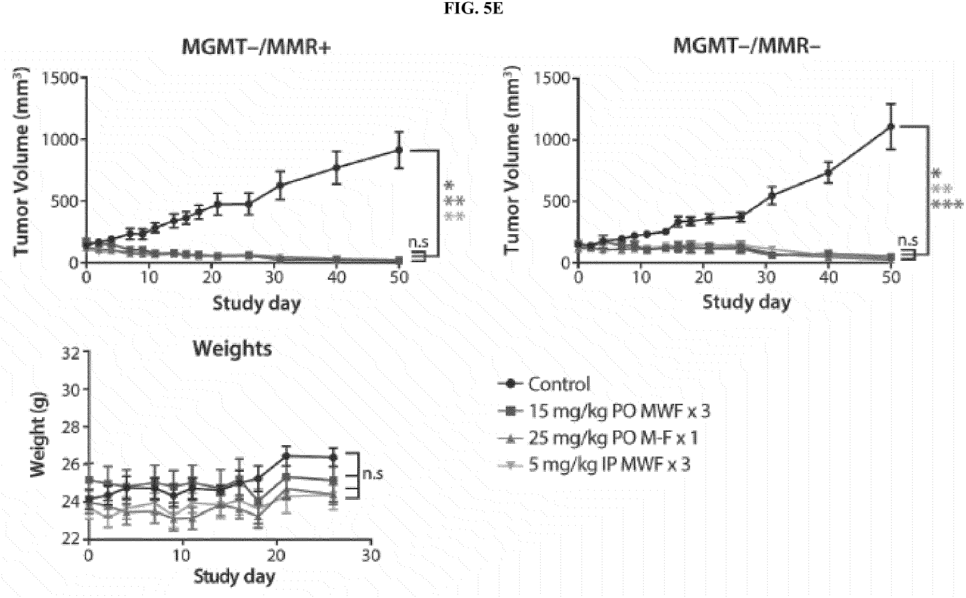

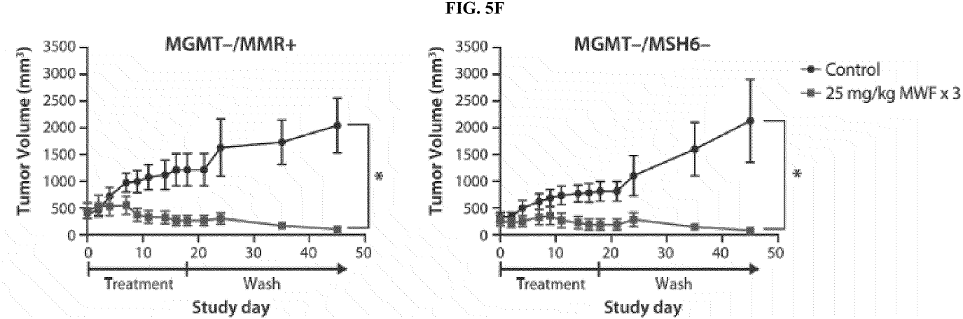

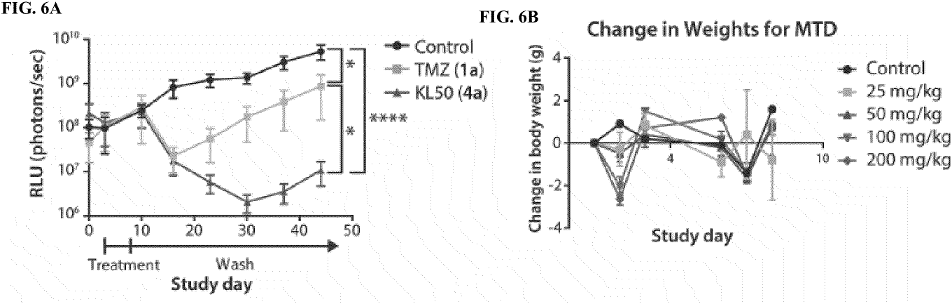

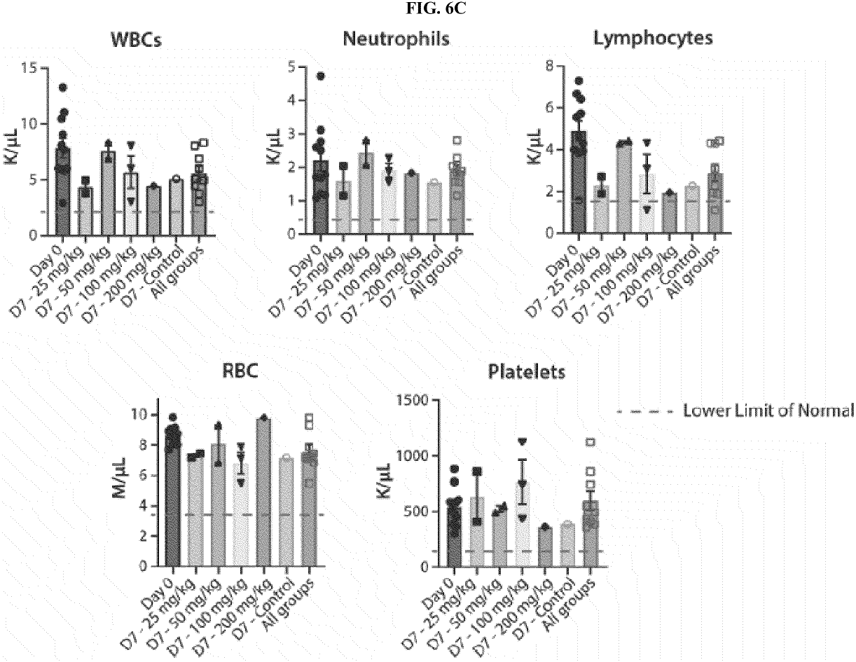

The activity of KL-50 (4a) and TMZ (1a) was evaluated in vivo using murine flank tumor models derived from the isogenic LN229 MGMT- cell lines. MGMT-/MMR+ and MGMT-/MMR- flank tumors were treated with KL-50 (4a) or TMZ (1a) (5 mg/kg MWF × 3 weeks) as previously described for TMZ (1a). TMZ (1a) suppressed tumor growth in the MGMT-/MMR+ tumors (FIG. 5A). KL-50 (4a) was statistically non-inferior to TMZ (1a), despite a 17% lower molar dosage owing to its higher molecular weight. In the MGMT-/MMR- tumors, TMZ (1a) demonstrated no efficacy, while KL-50 (4a) potently suppressed tumor growth (FIG. 5B). KL-50 (4a) treatment resulted in no significant changes in body weight compared to TMZ (1a) or control (FIG. 5C). Representative Kaplan-Meier survival curves are shown in FIG. 5D with a greater than 5-week increase in median OS for KL-50 (4a) vs TMZ (1a). KL-50 (4a) was effective and non-toxic using different dosing regimens (5 mg/kg, 15 mg/kg, 25 mg/kg), treatment schedules (MWF × 3 weeks, M-F × 1 week), and routes of drug administration (PO, IP) in mice bearing MGMT -/MMR + and MGMT-/MMR- flank tumors (FIG. 5E). KL-50 (4a; 25 mg/kg PO MWF × 3 weeks) potently suppressed the growth of large (~350-400 mm³) MGMT-/MMR+ and MGMT-/MSH6- tumors (FIG. 5F). KL-50 (4a; 25 mg/kg IP M-F × 1 week) was also effective in an orthotropic, intracranial LN229 MGMT-/MMR- model, whereas TMZ (1a) only transiently suppressed tumor growth (FIG. 6A).

A focused maximum tolerated dose study revealed KL-50 (4a) is well-tolerated. Healthy mice were treated with escalating doses of KL-50 (4a) (0, 25, 50, 100, and 200 mg/kg × 1 dose), and monitored over time for changes in both weights and hematologic profiles. Mice in the higher dosage groups (100 or 200 mg/kg) experienced a greater than 10% weight loss after treatment administration, which regressed to baseline at the end of one week (FIG. 6B). Two of three mice in the 200 mg/kg treatment group became observably ill warranting euthanasia, but no evidence of toxicity was observed in the remaining cohorts. As the main dose limiting systemic toxicity of TMZ (1a) is myelosuppression, complete blood counts were measured for all mice on day 0 before treatment and subsequently on day 7 after drug administration. Overall, neutrophils and lymphocytes experienced the most significant drops in cell count, although all blood counts were within normal physiological ranges (defined as values falling within 2 SDs of the average for healthy mice) for all cohorts (FIG. 6C). Taken together, these data demonstrate the robust in vivo efficacy, systemic tolerability, and CNS penetrance of KL-50 (4a).

### Example 14

Without being bound by theory, the successful therapeutic application of the compounds described herein arises from certain factors. In one aspect, following on the seminal clinical studies of Stupp and co-workers, who established MGMT expression as a predictive biomarker for TMZ (1a) treatment, MGMT silencing (which occurs in ~50% of GBMs and ~70% of grade II/III gliomas) was leveraged to obtain tumor cell selectivity. In one aspect, and in a departure from prior studies, bifunctional agents that are specifically designed to evolve slowly to ICLs following transfer to O⁶G were utilized, thereby establishing an MMR-independent method to amplify the therapeutic impact of MGMT silencing.

This strategy has led to a new class of agents for treatment of MGMT- glioma independent of MMR status. MMR mutation-induced alkylator resistance has been a major barrier to treatment efficacy, likely since the introduction of TMZ (1a) into glioma treatment regimens in the early 1990s. Bifunctional alkylation agents, such as lomustine (14) and MTZ (12a), have been tested with the hopes of overcoming TMZ (1a) resistance over the last ~30 years, but these agents lack a therapeutic index owing to their activity in MGMT+ (normal tissue) cells.

Literature data supports the notion that the remarkable cell line selectivity of KL-50 (4a) derives strictly from the poor leaving group ability of fluoride. While the aliphatic C—F bond is strong (~109 kcal/mol) and not normally susceptible to cleavage by bimolecular nucleophilic displacement, the appropriate positioning of hydrogen bond donors or covalently attached nucleophiles can promote substitution. The half-lives of O⁶-(2-fluoroethyl)guanosine (S1) and O⁶-(2-chloroethyl)guanosine (S4) are ~18.5 h and ~ 18 min, respectively, at 37° C. and pH 7.4 (FIGS. 7A and 7B). Intramolecular halide displacement gives the common intermediate N1,O⁶-ethanoguanosine (S2) which undergoes ring opening attack by water to yield N1-(2-hydroxyethyl)guanosine (S3). By comparison, attempts to hydrolyze N7-(2-fluoroethyl)guanosine (S5) to N7-(2-hydroxyethyl)guanosine (S6) in aqueous buffer (pH 7) at 37° C. were reportedly unsuccessful, likely due to an inability to form a similar cationic cyclized intermediate (FIG. 7C). Thus, while O⁶FEtG (5) lesions likely only constitute a small fraction of alkylation products derived from KL-50 (4a), it is possible that the more prevalent sites of alkylation, such as N7G, do not form ICLs at a significant rate, and are readily resolved. The intramolecular displacement pathway (5→6) provides an essential acceleration in the formation of ICLs by KL-50 (4a) to biologically relevant timescales and enables an ample kinetic window for MGMT-mediated repair of the primary O⁶FEtG (5) adduct.

These data also provide an explanation for the failure of related cross-linking agents to display the therapeutic index that underpins TMZ’s (1a) success. 2-Chloroethyl nitrosoureas (e.g., lomustine, 14) or 2-chloroethylimidazotetrazines (e.g., MTZ, 12a) are known to form ICLs such as 8 by a pathway analogous to KL-50 (4a, see FIG. 1E). However, they can also generate ICLs via direct chloride displacement from 2-chlorethyl adducts present at other sites of DNA alkylation, which degrades the therapeutic index of these compounds. Consistent with this, the time-course analysis established a faster onset of ICLs for MTZ (12a) than KL-50 (4a), and, in turn, explains their differential MGMT selectivity (~7-fold and ~25-fold for 12a and 4a, respectively).

An order-of-estimate calculation provides insight into the number of ICLs necessarily generated by KL-50 (4a) to induce toxicity. It has been reported that the mean lethal dose of ICLs in HeLa cells is 230 and TMZ (1a) has been demonstrated to yield 0,600 O⁶MeG (3) adducts per cell at a dose of 20 µM. Assuming a similar level of O⁶FEtG (5) lesions are induced by KL-50 (4a) at the IC₅₀ (~20 µM) in MGMT-/MMR- LN229 cells, the number of adducts required to convert to ICLs to generate the mean lethal dose is ~1 in 90, or ~1.1% cross-linking efficiency.

Extensive characterization of KL-50 (4a) versus TMZ (1a) activity in vitro was performed to support selectively targeting MGMT- cells independent of MMR status. While MGMT-/MMR- cells display no signs of DNA damage or DNA repair signaling in response to TMZ (1a), MMR-independent, activation of DNA damage checkpoint signaling, DNA repair foci formation, cell cycle arrest, and micronuclei formation was found following KL-50 (4a) treatment. Moreover, KL-50 (4a) retained its effectiveness in vivo in MMR-deficient flank and intracranial tumor models resistant to TMZ (1a) as well as in large MSH6-deficient tumors, a commonly lost MMR component reported in glioma patients.

Beyond MGMT-silenced recurrent glioma, other potential beneficial indications for selective targeting of cancer cells with KL-50 (4a) are possible, according to various embodiments. MGMT silencing has been reported in 40% of colorectal cancers and 25% of non-small cell lung cancer, lymphoma, and head & neck cancers. MGMT mRNA expression is also reduced in subsets of additional cancer types, including breast carcinoma, bladder cancer, and leukemia. MMR loss, as reported by microsatellite instability, is a well-established phenomenon in multiple cancer types and leads to resistance to various standard of care agents. It therefore stands to reason that there are likely other subsets of MGMT-/MMR- tumors in both initial and recurrent settings that would be ideal targets for KL-50 (4a).

The data also suggest KL-50 (4a) will display a higher therapeutic index in tumors with MGMT deficiency and impaired ICL repair, including HR deficiency. It was demonstrated that FANCD2- and BRCA2-deficient cells are hypersensitive to KL-50 (4a), particularly in the setting of MGMT depletion. Remarkably, the therapeutic index (TI) of KL-50 (4a) in the DLD1 isogenic model, as measured by the ratio of IC₅₀ values in MGMT+/BRCA2+ cells compared to MGMT-/BRCA2- cells, was ~600-fold, vastly larger than canonical crosslinking agents such as cisplatin (42-fold) or MMC (26-fold). A similar amplification of the TI was seen in the PEO1/4 model with KL-50 (4a) (62-fold) vs. cisplatin (13-fold) or MMC (7-fold). HR-related gene mutations have been detected in a substantial number of tumors across multiple cancer types (17.4% in 21 cancer lineages) and methods have been developed to assess for tumor-associated HR deficiency. Thus, in the modern era of molecular precision medicine, the biomarker-guided use of KL-50 (4a) in individual cancers could result in therapeutic indices and exquisite tumor sensitivities previously only observed with synthetic lethal interactions targeting DNA repair proteins. Finally, many possibilities are envisioned for combination studies of KL-50 (4a) with DNA repair inhibitors such as checkpoint kinase inhibitors or potentially immunotherapy in the setting of MMR mutations.

These findings may have, in various embodiments, profound clinical implications for patients with recurrent MGMT-methylated glioma, of which up to half acquire TMZ (1a) resistance via loss of MMR. As demonstrated by the analysis of related TMZ (1a) derivatives, KL-50 (4a) is uniquely designed to fill this therapeutic void. In addition, because KL-50 (4a) may be rapidly phased into clinical trials and readily amenable to derivatization for improved drug pharmacokinetic properties, such enhanced as CNS penetration, based on prior work with the imidazotetrazine scaffold. More broadly, incorporating the rates of DNA modification and DNA repair pathways in therapeutic design strategies may lead to the development of additional selective chemotherapies.

### Example 15: Anticancer Activity Towards Human Brain Glioblastoma Cells

Exemplary compounds were evaluated for activity against LN229 human brain glioblastoma cells. Experimental procedures and results are provided below.

### Part I - Experimental Procedure

LN229 quad cells were maintained in Dulbecco’s Modified Eagle Medium (DMEM) supplemented with 10% fetal bovine serum. Cells were plated into sterile black with glass bottom 384-well plates (Cellvis) at a concentration of 1,000 cells/well (20 µL total volume) using a MultiDrop (Thermo Fisher). Then, assay plates were centrifuged at 300 rpm for 2 seconds and incubated overnight in a 37° C. 5% CO₂ incubator. Compounds were prepared as 100 mM stocks in DMSO and stored protected from light at -20′C until use. Prior to compound addition, the stock solutions of compound were diluted two-fold serially in DMSO from 100 mM down to 0.05 mM in a 384-well Source plate. Vehicle control wells containing DMSO and positive control wells containing 10 mM bortezomib were also added to the Source plate. An aliquot in the amount of 40 nL of compound stock solution or DMSO vehicle was transferred from the source plate to the cell assay plate using an Echo Acoustic dispense (Beckman). Three replicate dilution curves of each compound were run on each assay plate. An aliquot in the amount of 120 nL of a 10 mM bortezomib solution was transferred to the positive control wells resulting in a final concentration of 60 µM. The final concentration of compounds ranged from 200 µM to 0.1 µM (12-point, 2-fold dilution dose response curve), and the final DMSO concentration was 0.2%.

Assay plates were centrifuged at 500 rpm for 2 seconds and incubated for 120 hours at 37° C. in a humidified 5% CO₂ incubator. Following incubation, cells were fixed with 4% paraformaldehyde and stained with DAPI for nuclei visualization. The images were acquired on the InCell 2200 high content imager (GE, now Molecular Devices), and quantified using InCell Analyzer image analysis software. Z prime factor, signal-to-background and coefficient of variation were calculated for each assays plate using mean and standard deviation values of the negative (vehicle) and positive (bortezomib) control wells to ensure assay robustness. Raw cell count data for test compounds was normalized to percent viability relative to the DMSO vehicle control. Data was plotted in GraphPad Prism using a variable slope 4-parameter fit.

### Part II - Results

Inhibition data for compounds tested in the assay is provided in Table 4 below. The symbol “+++” indicates a IC₅₀ less than 50 µM. The symbol “++” indicates a IC₅₀ in the range of 50 µM to 90 µM. The symbol “+” indicates a IC₅₀ greater than 90 µM.

TABLE 4

| | | | | |
|---|---|---|---|---|
| Compound | MGMT+ MMR+ Activity | MGMT+ MMR -Activity | MGMT -MMR + Activity | MGMT -MMR-Activity |
| 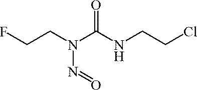 [SMILES: O=NN(CCF)C(=O)NCCCl] | + | + | ++ | +++ |
| 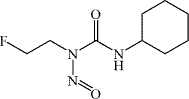 [SMILES: O=NN(CCF)C(=O)NC1CCCCC1] | + | ++ | +++ | +++ |
| 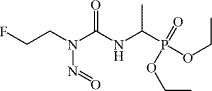 [SMILES: CCOP(=O)(OCC)C(C)NC(=O)N(CCF)N=O] | + | + | + | +++ |
| 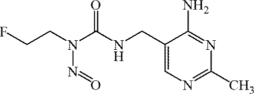 [SMILES: Cc1ncc(CNC(=O)N(CCF)N=O)c(N)n1] | + | + | + | +++ |

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A method for treating, ameliorating, or preventing an MGMT deficient cancer in a patient in need thereof, the method comprising administering to the patient a therapeutically-effective amount of a compound of formula (I), or a pharmaceutically-acceptable salt thereof: 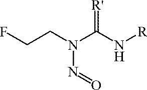
wherein:
R′ is selected from the group consisting of O and NH; and
R is selected from the group consisting of H, NO₂, CH₂CH₂Cl, CH₂CH₂F, cyclohexyl,
4-methylcyclohexyl, —C(H)(CH₃)—P(═O)(OCH₂CH₃)₂, 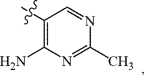 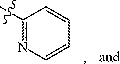 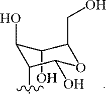
.

2. The method of claim 1, wherein the cancer is MMR deficient.

3. The method of claim 1, wherein the compound of formula (I) is selected from the group consisting of: 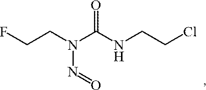 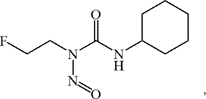 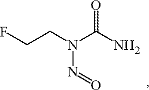 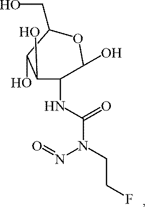 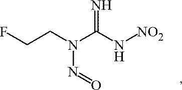 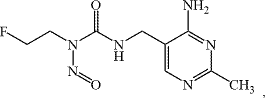 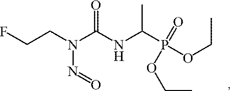 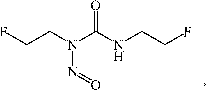 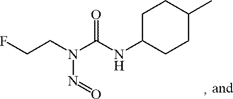 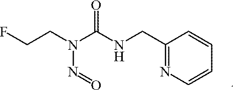
.

4. The method of claim 1, wherein the cancer is selected from the group consisting of urothelial cancer, breast invasive carcinoma, colon adenocarcinoma, head and neck tumor (SCC), lung adenocarcinoma, rectum adenocarcinoma, acute myeloid leukemia, glioblastoma multiforme, brain lower grade glioma, Hodgkin’s lymphoma, non-Hodgkin’s lymphoma, melanoma, and lung cancer.

5. The method of claim 4, wherein the cancer is MMR deficient.

6. The method of claim 5, wherein the cancer is glioblastoma multiforme.

7. A pharmaceutical composition comprising a compound of formula (I), or a pharmaceutically-acceptable salt thereof, and at least one pharmaceutically-acceptable carrier or excipient: 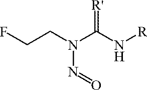
wherein:
R′ is selected from the group consisting of O and NH; and
R is selected from the group consisting of H, NO₂, C₂H₄Cl, C₂H₄F, cyclohexyl, 4-
methylcyclohexyl, —C(H)(CH₃)—P(═O)(OCH₂CH₃)₂, 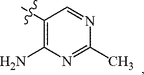 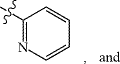 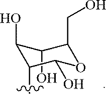
.

8. The composition of claim 7, which comprises a therapeutically-effective cancer-treatment amount of the compound of formula (I).

9. The composition of claim 8, wherein the cancer to be treated is selected from the group consisting of urothelial cancer, breast invasive carcinoma, colon adenocarcinoma, head and neck tumor (SCC), lung adenocarcinoma, rectum adenocarcinoma, acute myeloid leukemia, glioblastoma multiforme, brain lower grade glioma, Hodgkin’s lymphoma, non-Hodgkin’s lymphoma, and melanoma.

10. The composition of claim 7, wherein the compound of formula (I) is selected from the group consisting of: 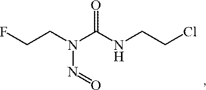 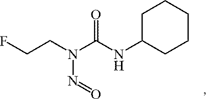 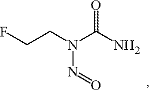 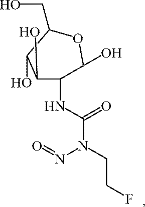 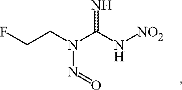 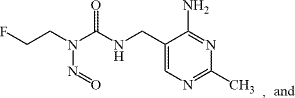 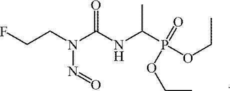
.

11. A compound of formula (I), or a pharmaceutically-acceptable salt thereof: 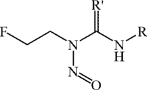
wherein:
R′ is selected from the group consisting of O and NH; and
R is selected from the group consisting of NO₂, —C(H)(CH₃)—P(═O)(OCH₂CH₃)₂, 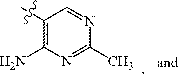 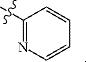
.

12. The composition of claim 10, which is selected from the group consisting of: 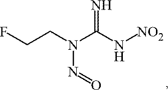 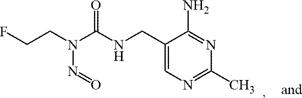 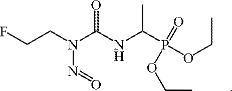
.
